(19) United States
(12) Patent Application Publication
(10) Pub. No.: US 2021/0361761 A1
(43) Pub. Date: Nov. 25, 2021

(54) NOVEL YELLOW FEVER NUCLEIC ACID MOLECULES FOR VACCINATION

(71) Applicant: CureVac AG, Tübingen (DE)

(72) Inventors: Johannes LUTZ, Tübingen (DE); Susanne RAUCH, Tübingen (DE); Regina HEIDENREICH, Tübingen (DE); Benjamin PETSCH, Tübingen (DE)

(73) Assignee: CureVac AG, Tübingen (DE)

(21) Appl. No.: 17/045,236

(22) PCT Filed: Apr. 5, 2019
(86) PCT No.: PCT/EP2019/058709

§ 371 Date: Oct. 5, 2020

(30) Foreign Application Priority Data
Apr. 5, 2018 (EP) PCT/EP2018/058784

(51) Int. Cl. A61K 39/12; C12N 7/00; A61K 9/51

(52) U.S. Cl. CPC A61K 39/12; C12N 7/00; A61K 2039/53; C12N 2770/24134; A61K 9/5123

(57) ABSTRACT

The present invention is directed to an artificial nucleic acid, particularly to an artificial RNA suitable for use in treatment and/or prophylaxis of an infection with yellow fewer vims (YFV) or a disorder related to such an infection. The invention further concerns a method of treating or preventing a disorder or a disease, first and second medical uses of the artificial RNA, compositions and vaccines. Further, the invention is directed to a kit, particularly to a kit of parts, comprising the artificial RNA, compositions and vaccines.

### INTRODUCTION

The present invention is directed to artificial RNA suitable for use in the treatment or prophylaxis of an infection with yellow fever virus (YFV) or of a disorder related to such an infection. In particular, the artificial RNA of the invention comprises at least one heterologous untranslated region (UTR), preferably a 3′-UTR and/or a 5′-UTR, and a coding region encoding at least one antigenic peptide or protein derived from YFV, in particular at least one antigenic peptide or protein derived from YFV prME polyprotein and/or the non-structural protein NS1. The artificial RNA is preferably characterized by increased expression efficacies of coding regions operably linked to said UTR elements. The present invention is also directed to compositions and vaccines comprising said artificial RNA in association with a polymeric carrier, a polycationic protein or peptide, or a lipid nanoparticle (LNP). Further, the invention concerns a kit, particularly a kit of parts comprising the artificial RNA or composition or vaccine. The invention is further directed to a method of treating or preventing a disorder or a disease, and first and second medical uses of the artificial RNA, composition, or vaccine.

Yellow fever virus (YFV) is a Flavivirus, a group of enveloped positive-stranded RNA arboviruses. Among the flaviviruses there are more than 40 human pathogens responsible for considerable morbidity and mortality throughout the world causing symptoms ranging from rather unspecific pseudo-flu-like syndromes, to severe encephalitic or hemorrhagic disease. YFV is endemic in tropical and subtropical regions in Africa and South-America and causes epidemics of hemorrhagic fever with high fatality rates from 20-50% resulting in an estimated number of 200,000 cases with 30,000 deaths annually.

In the 1930s, a live attenuated Yellow fever vaccine virus (17D) was developed which confers long-term immunity upon a single injection. However, in some cases vaccination with the 17D YF vaccine may elicit severe side effects such as anaphylactic reactions and yellow-fever-vaccine-associated neurologic disease (YEL-AND). Anaphylaxis is most likely caused by allergic reactions to proteins from eggs or gelatine used in vaccine production. The fatality associated with YEL-AND appears to be relatively low in general, but higher among recipients 60 years of age or older and is presumably attributed to the injection of a live attenuated virus into recipients who fail to adequately control the replication of virus. Other risks associated with the use of the live attenuated YF vaccine are transmission of the 17D virus through transfusion of blood products from recently vaccinated donors and vertical mother-to-child transmission.

A further complication associated with live attenuated vaccines is Yellow fever vaccine-associated viscerotropic disease (YEL-AVD). YEL-AVD is an illness similar to wild-type yellow fever, in which the vaccine virus proliferates in multiple organs, causing multiple organ dysfunction syndrome or multiorgan failure and death in at least 60% of cases. Viscerotropic disease has been reported in primary vaccines only, with average onset 4 days (range: 0-8 days) after vaccination.

Therefore, a safe and effective, non-infectious vaccine would be desirable in order to avoid vaccine-associated adverse events and to allow vaccination of young infants and immunocompromised recipients, for whom the live 17D vaccine is contraindicated, as well as pregnant and nursing women and elderly people.

Further it would be desirable that a yellow fever vaccine has some of the following advantageous features:
Very efficient induction of YFV antigen-specific immune responses against the encoded antigenic peptide or protein at a very low dosages and dosing regimen.
Induction of a YFV-specific strong humoral immune response
Induction of YFV-specific B-cell memory
Faster onset of immune protection against YFV
Longevity of the induced immune responses against YFV
Induction of broad cellular T-cell responses against YFV
Induction of a (local and transient) pro-inflammatory environment
No induction of systemic cytokine or chemokine response after application of the vaccine
Well tolerability, no side-effects, non toxic
No induction of Yellow Fever Vaccine-Associated Neurologic Disease (YEL-AND) and Yellow Fever Vaccine-Associated Viscerotropic Disease (YEL-AVD))
Reduction of the YFV pathogenesis by lowering vascular leakage
Broadening the immune response by targeting two different key antigens, e.g. envelope and NS1
Advantageous stability characteristics of the vaccine
Speed, adaptability, simplicity and scalability of YFV vaccine production

Accordingly, there remains an unmet medical need to provide safe and effective YFV vaccine suitable for pre-exposure prophylaxis and post-exposure prophylaxis. Moreover, there is a need for the development of a safe and effective YFV vaccine that is affordable, that can be manufactured using a scalable, cost-effective, and fast-adaptable production process, and which preferably has superior characteristics in terms of stability (e.g. heat stability).

It is an object of the underlying invention to provide artificial RNA encoding at least one antigenic peptide or protein derived from YFV characterized by increased expression efficacies which is a prerequisite of an effective RNA-based vaccine. A further object of the underlying invention is to provide safe and effective compositions and vaccines against YFV which are preferably suitable for pre-exposure prophylaxis or for post-exposure prophylaxis.

The objects outlined above are solved by the claimed subject matter.

### Definitions

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as percentages by weight (wt.-%).

Adaptive immune response: The term “adaptive immune response” as used herein will be recognized and understood by the person of ordinary skill in the art, and is for example intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells). In the context of the invention, the antigen is provided by the artificial RNA coding sequence encoding at least one antigenic peptide or protein.

Antigen: The term “antigen” as used herein will be recognized and understood by the person of ordinary skill in the art, and is for example intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides, or proteins derived from YFV prME polyprotein or from the non-structural protein NS1 comprising at least one epitope are understood as antigens in the context of the invention. In the context of the present invention, an antigen may be the product of translation of a provided artificial RNA as specified herein.

Antigenic peptide or protein: The term “antigenic peptide or protein” will be recognized and understood by the person of ordinary skill in the art, and is for example intended to refer to a peptide, protein (or polyprotein) derived from a (antigenic) protein/polyprotein which may stimulate the body's adaptive immune system to provide an adaptive immune response. Therefore an “antigenic peptide or protein” comprises at least one epitope (as defined herein) or antigen (as defined herein) of the protein it is derived from (e.g., in the context of the invention, YFV peptide or protein, preferably YFV prME).

Artificial nucleic acid: The terms “artificial nucleic acid” as used herein will be recognized and understood by the person of ordinary skill in the art, and are for example intended to refer to an artificial nucleic acid that does not occur naturally. An artificial nucleic acid may be a DNA molecule, an RNA molecule or a hybrid-molecule comprising DNA and RNA portions. Typically, artificial nucleic acids may be designed and/or generated by genetic engineering methods to correspond to a desired artificial sequence of nucleotides (heterologous sequence). In this context an artificial sequence is usually a sequence that may not occur naturally, i.e. it differs from the wild type sequence by at least one nucleotide. The term “wild type” as used herein will be recognized and understood by the person of ordinary skill in the art, and is for example intended to refer to a sequence occurring in nature. Further, the term “artificial nucleic acid” is not restricted to mean “one single molecule” but is, typically, understood to comprise an ensemble of essentially identical molecules.

Artificial RNA: The term “artificial RNA” as used herein is intended to refer to an RNA that does not occur naturally. In other words, an artificial RNA may be understood as a non-natural nucleic acid molecule. Such RNA molecules may be non-natural due to its individual sequence (which does not occur naturally, e.g. G/C content modified coding sequence, UTRs) and/or due to other modifications, e.g. structural modifications of nucleotides which do not occur naturally. Typically, artificial RNA may be designed and/or generated by genetic engineering methods to correspond to a desired artificial sequence of nucleotides (heterologous sequence). In this context an artificial RNA sequence is usually a sequence that may not occur naturally, i.e. it differs from the wild type sequence by at least one nucleotide. The term “artificial RNA” is not restricted to mean “one single molecule” but is, typically, understood to comprise an ensemble of essentially identical molecules. Accordingly, it may relate to a plurality of essentially identical RNA molecules contained in an aliquot or a sample.

Cationic: Unless a different meaning is clear from the specific context, the term “cationic” means that the respective structure bears a positive charge, either permanently or not permanently but in response to certain conditions such as pH. Thus, the term “cationic” covers both “permanently cationic” and “cationisable”.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. For example, in some embodiments, if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In other embodiments, it is preferred that the cationisable compound or moiety is predominantly neutral at physiological pH values, e.g. about 7.0 to 7.4, but becomes positively charged at lower pH values. In some embodiments, the preferred range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are for example intended to refer to a sequence of several nucleotide triplets, which may be translated into a peptide or protein. A coding sequence in the context of the present invention is preferably an RNA sequence, consisting of a number of nucleotides that may be divided by three, which starts with a start codon and which preferably terminates with a stop codon.

Composition: In the context of the invention, a “composition” refers to any type of composition in which the specified ingredients (e.g. artificial RNA of the invention in association with LNP), may be incorporated, optionally along with any further constituents, usually with at least one pharmaceutically acceptable carrier or excipient. Thus, the composition may be a dry composition such as a powder or granules, or a solid unit such as a lyophilized form or a tablet. Alternatively, the composition may be in liquid form, and each constituent may be independently incorporated in dissolved or dispersed (e.g. suspended or emulsified) form.

Compound: As used herein, a “compound” means a chemical substance, which is a material consisting of molecules having essentially the same chemical structure and properties. For a small molecular compound, the molecules are typically identical with respect to their atomic composition and structural configuration. For a macromolecular or polymeric compound, the molecules of a compound are highly similar but not all of them are necessarily identical. For example, a segment of a polymer that is designated to consist of 50 monomeric units may also contain individual molecules with e.g. 48 or 53 monomeric units.

Derived from: The term “derived from” as used throughout the present specification in the context of a nucleic acid, i.e. for a nucleic acid “derived from” (another) nucleic acid, means that the nucleic acid, which is derived from (another) nucleic acid, shares at least 50%, preferably at least 55%, preferably at least 60%, preferably at least 65%, preferably at least 70%, more preferably at least 75%, more preferably at least 80%, 81%, 82%, 83%, 84%, more preferably at least 85%, 86%, 87%, 88%, 89% even more preferably at least 90%, 91%, 92%, 93%, 94%, even more preferably at least 95%, 96%, 97%, and particularly preferably at least 98%, 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the thymidines (T) by uracils (U) throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. It goes without saying that such modifications are preferred, which do not impair RNA stability, e.g. in comparison to the nucleic acid from which it is derived. In the context of amino acid sequences (e.g. antigenic peptides or proteins) the term “derived from” means that the amino acid sequence, which is derived from (another) amino acid sequence (e.g. YFV prME or YFV NS1), shares at least 50%, preferably at least 55%, preferably at least 60%, preferably at least 65%, preferably at least 70%, more preferably at least 75%, more preferably at least 80%, 81%, 82%, 83%, 84%, more preferably at least 85%, 86%, 87%, 88%, 89% even more preferably at least 90%, 91%, 92%, 93%, 94%, even more preferably at least 95%, 96%, 97%, and particularly preferably at least 98%, 99% sequence identity with the amino acid sequence from which it is derived. Thus, it is understood, if a antigenic peptides or protein is “derived from” a YFV protein, e.g. prME, the antigenic peptides or protein that is “derived from” said YFV prME may differ represent a variant or fragment of the (full length) prME protein (e.g. a variant or fragment of pr, M, E, prM, prME or any combinations thereof). Moreover, the antigenic peptides or protein that is “derived from” said YFV prME may differ in the amino acid sequence, sharing a certain percentage of identity as defined above.

Epitope: The term “epitope” (also called “antigen determinant” in the art) as used herein will be recognized and understood by the person of ordinary skill in the art, and is for example intended to refer to T cell epitopes and B cell epitopes. T cell epitopes or parts of the antigenic peptides or proteins may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 to about 20 or even more amino acids, wherein these fragments may be selected from any part of the amino acid sequence. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens, preferably having 5 to 15 amino acids, more preferably having 5 to 12 amino acids, even more preferably having 6 to 9 amino acids, which may be recognized by antibodies, i.e. in their native form. Such epitopes of proteins or peptides may furthermore be selected from any of the herein mentioned variants of such proteins or peptides. In this context antigenic determinants can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain. In the context of the present invention, an epitope may be the product of translation of a provided artificial RNA as specified herein.

Fragment: The term “fragment” as used throughout the present specification in the context of a nucleic acid sequence or an amino acid sequence may typically be a shorter portion of a full-length sequence of e.g. a nucleic acid sequence or an amino acid sequence. Accordingly, a fragment, typically, consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 5%, 10%, 20%, preferably at least 30%, more preferably at least 40%, more preferably at least 50%, even more preferably at least 60%, even more preferably at least 70%, and most preferably at least 80% of the total (i.e. full-length) molecule from which the fragment is derived (e.g. YFV prME or YFV NS1). The term “fragment” as used throughout the present specification in the context of proteins or peptides may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide. In the context of antigens such fragment may have a length of about 6 to about 20 or even more amino acids, e.g. fragments as processed and presented by MHC class I molecules, preferably having a length of about 8 to about 10 amino acids, e.g. 8, 9, or 10, (or even 6, 7, 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T-cells in form of a complex consisting of the peptide fragment and an MHC molecule, i.e. the fragments are typically not recognized in their native form. Fragments of proteins or peptides (e.g. in the context of antigens) may comprise at least one epitope of those proteins or peptides. Furthermore also domains of a protein, like the extracellular domain, the intracellular domain or the transmembrane domain and shortened or truncated versions of a protein may be understood to comprise a fragment of a protein.

Heterologous: The terms “heterologous” or “heterologous sequence” as used throughout the present specification in the context of a nucleic acid sequence or an amino acid sequence refers to a sequence (e.g. DNA, RNA, amino acid) will be recognized and understood by the person of ordinary skill in the art, and is intended to refer to a sequence that is derived from another gene, from another allele, from another species. Two sequences are typically understood to be “heterologous” if they are not derivable from the same gene or in the same allele. I.e., although heterologous sequences may be derivable from the same organism, they naturally (in nature) do not occur in the same nucleic acid molecule, such as e.g. in the same RNA, or the same protein.

Humoral immune response: The terms “humoral immunity” or “humoral immune response” will be recognized and understood by the person of ordinary skill in the art, and are for example intended to refer to B-cell mediated antibody production and optionally to accessory processes accompanying antibody production. A humoral immune response may be typically characterized, e.g. by Th2 activation and cytokine production, germinal center formation and isotype switching, affinity maturation and memory cell generation. Humoral immunity also typically may refer to the effector functions of antibodies, which include pathogen and toxin neutralization, classical complement activation, and opsonin promotion of phagocytosis and pathogen elimination.

Identity (of a sequence): The term “identity” as used throughout the present specification in the context of a nucleic acid sequence or an amino acid sequence will be recognized and understood by the person of ordinary skill in the art, and is for example intended to refer to the percentage to which two sequences are identical. To determine the percentage to which two sequences are identical, e.g. nucleic acid sequences or amino acid sequences as defined herein, preferably the amino acid sequences encoded by the artificial nucleic acid sequence as defined herein or the amino acid sequences themselves, the sequences can be aligned in order to be subsequently compared to one another. Therefore, e.g. a position of a first sequence may be compared with the corresponding position of the second sequence. If a position in the first sequence is occupied by the same component (residue) as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using a mathematical algorithm. A preferred, but not limiting, example of a mathematical algorithm which can be used is the algorithm is integrated in the BLAST program. Sequences which are identical to the sequences of the present invention to a certain extent can be identified by this program.

Immunogen, immunogenic: The terms “immunogen” or “immunogenic” will be recognized and understood by the person of ordinary skill in the art, and are for example intended to refer to a compound that is able to stimulate/induce an immune response. Preferably, an immunogen is a peptide, polypeptide, or protein. An immunogen in the sense of the present invention is the product of translation of a provided artificial nucleic acid, preferably RNA, comprising at least one coding sequence encoding at least one antigenic peptide, protein derived from YFV as defined herein. Typically, an immunogen elicits an adaptive immune response.

Immune response: The term “immune response” will be recognized and understood by the person of ordinary skill in the art, and is for example intended to refer to a specific reaction of the adaptive immune system to a particular antigen (so called specific or adaptive immune response) or an unspecific reaction of the innate immune system (so called unspecific or innate immune response), or a combination thereof.

Immune system: The term “immune system” will be recognized and understood by the person of ordinary skill in the art, and is for example intended to refer to a system of the organism that may protect the organisms from infection. If a pathogen succeeds in passing a physical barrier of an organism and enters this organism, the innate immune system provides an immediate, but non-specific response. If pathogens evade this innate response, vertebrates possess a second layer of protection, the adaptive immune system. Here, the immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts typically contains so called humoral and cellular components.

Innate immune system: The term “innate immune system” (also known as non-specific or unspecific immune system) will be recognized and understood by the person of ordinary skill in the art, and is for example intended to refer to a system typically comprising the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be, e.g. activated by ligands of Toll-like receptors (TLRs) or other auxiliary substances such as lipopolysaccharides, TNF-alpha, CD40 ligand, or cytokines, monokines, lymphokines, interleukins or chemokines, IL-1 to IL-33, IFN-alpha, IFN-beta, IFN-gamma, GM-CSF, G-CSF, M-CSF, LT-beta, TNF-alpha, growth factors, and hGH, a ligand of human Toll-like receptor (e.g., TLR1 to TLR10), a ligand of murine Toll-like receptor, (e.g., TLR1 to TLR13), a ligand of a NOD-like receptor, a ligand of a RIG-I like receptor, an immunostimulatory nucleic acid, an immunostimulatory RNA (isRNA), a CpG-DNA, an antibacterial agent, or an anti-viral agent.

Lipidoid compound: A lipidoid compound, also simply referred to as lipidoid, is a lipid-like compound, i.e. an amphiphilic compound with lipid-like physical properties. In the context of the present invention the term lipid is considered to encompass lipidoid compounds.

Nucleic acid: The terms “nucleic acid” or “nucleic acid molecule” will be recognized and understood by the person of ordinary skill in the art, and are for example intended to refer to a molecule comprising, preferably consisting of nucleic acid components. The term nucleic acid molecule preferably refers to DNA or RNA molecules. It is preferably used synonymous with the term polynucleotide. Preferably, a nucleic acid or a nucleic acid molecule is a polymer comprising or consisting of nucleotide monomers, which are covalently linked to each other by phosphodiester-bonds of a sugar/phosphate-backbone. The term “nucleic acid molecule” also encompasses modified nucleic acid molecules, such as base-modified, sugar-modified or backbone-modified DNA or RNA molecules as defined herein.

Nucleic acid sequence/RNA sequence/amino acid sequence: The terms “nucleic acid sequence”, “RNA sequence” or “amino acid sequence” will be recognized and understood by the person of ordinary skill in the art, and are for example intended to refer to particular and individual order of the succession of its nucleotides or amino acids respectively.

Permanently cationic: The term “permanently cationic” as used herein will be recognized and understood by the person of ordinary skill in the art, and means, for example, that the respective compound, or group or atom, is positively charged at any pH value or hydrogen ion activity of its environment. Typically, the positive charge is results from the presence of a quaternary nitrogen atom. Where a compound carries a plurality of such positive charges, it may be referred to as permanently polycationic, which is a subcategory of permanently cationic.

Pharmaceutically effective amount: The terms “pharmaceutically effective amount” or “effective amount” will be recognized and understood by the person of ordinary skill in the art, and are for example intended to refer to an amount of a compound (e.g. the artificial RNA of the invention) that is sufficient to induce a pharmaceutical effect, such as, in the context of the invention, an immune response (e.g. against an antigenic peptide, protein, polyprotein as defined herein).

Stabilized nucleic acid molecule” or “stabilized RNA: The term “stabilized nucleic acid molecule” or “stabilized RNA” refer to a nucleic acid molecule, preferably an RNA molecule that is modified such, that it is more stable to disintegration or degradation, e.g., by environmental factors or enzymatic digest, such as by an exo- or endonuclease degradation, than the nucleic acid molecule without the modification. Preferably, a stabilized nucleic acid molecule, e.g. stabilized RNA, in the context of the present invention is stabilized in a cell, such as a prokaryotic or eukaryotic cell, preferably in a mammalian cell, such as a human cell. The stabilization effect may also be exerted outside of cells, e.g. in a buffer solution etc., for example, in a manufacturing process for a pharmaceutical composition comprising the stabilized nucleic acid molecule.

T-cell responses: The terms “cellular immunity” or “cellular immune response” or “cellular T-cell responses” as used herein will be recognized and understood by the person of ordinary skill in the art, and are for example intended to refer to the activation of macrophages, natural killer cells (NK), antigen-specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface. In the context of the invention, the antigen is provided by the artificial RNA encoding at least one antigenic peptide or protein derived from YFV, suitably inducing T-cell responses.

Variant (of a sequence): The term “variant” as used throughout the present specification in the context of a nucleic acid sequence will be recognized and understood by the person of ordinary skill in the art, and is for example intended to refer to a variant of nucleic acid sequences which forms the basis of a nucleic acid sequence. For example, a variant nucleic acid sequence may exhibit one or more nucleotide deletions, insertions, additions and/or substitutions compared to the nucleic acid sequence from which the variant is derived. Preferably, a variant of a nucleic acid sequence is at least 40%, preferably at least 50%, more preferably at least 60%, more preferably at least 70%, even more preferably at least 80%, even more preferably at least 90%, most preferably at least 95% identical to the nucleic acid sequence the variant is derived from. Preferably, the variant is a functional variant. A “variant” of a nucleic acid sequence may have at least 70%, 75%, 80%, 85%, 90%, 95%, 98% or 99% nucleotide identity over a stretch of 10, 20, 30, 50, 75 or 100 nucleotide of such nucleic acid sequence.

The term “variant” as used throughout the present specification in the context of proteins or peptides will be recognized and understood by the person of ordinary skill in the art, and is for example intended to refer to a proteins or peptide variant having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined in the context of the present invention may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of 10, 20, 30, 50, 75 or 100 amino acids of such protein or peptide. Preferably, a variant of a protein comprises a functional variant of the protein, which means that the variant exerts the same effect or functionality as the protein it is derived from.

3′-untranslated region, 3′-UTR element, 3′-UTR: The term “S-untranslated region” or “3′-UTR element” will be recognized and understood by the person of ordinary skill in the art, and are for example intended to refer to a part of a nucleic acid molecule, which is located 3′ (i.e. “downstream”) of a coding sequence and which is typically not translated into protein. Usually, a 3′-UTR is the part of an mRNA which is located between the coding sequence (CDS) and the poly(A) sequence of the mRNA. In the context of the invention, the term 3′-UTR may also comprise elements, which are not encoded in the DNA template, from which an artificial RNA is transcribed, but which are added after transcription during maturation, e.g. a poly(A) sequence.

5′-untranslated region, 5′-UTR element, 5′-UTR: The term “5′-untranslated region (5′-UTR)” will be recognized and understood by the person of ordinary skill in the art, and are for example intended to refer to a part of a nucleic acid molecule, which is located 5′ (i.e. “upstream”) of a coding sequence and which is not translated into protein. A 5′-UTR is typically understood to be a particular section of messenger RNA (mRNA), which is located 5′ of the coding sequence of the mRNA. Typically, the 5′-UTR starts with the transcriptional start site and ends one nucleotide before the start codon of the coding sequence. Preferably, the 5′-UTRs have a length of more than 20, 30, 40 or 50 nucleotides. The 5′-UTR may comprise elements for controlling gene expression, also called regulatory elements. Such regulatory elements may be, for example, ribosomal binding sites. The 5′-UTR may be post-transcriptionally modified, for example by addition of a 5′-cap.

5′ terminal oligopyrimidine tract (TOP), TOP-UTR: The term “5′ terminal oligopyrimidine tract (TOP)” has to be understood as a stretch of pyrimidine nucleotides located in the 5′ terminal region of a nucleic acid molecule, such as the 5′ terminal region of certain mRNA molecules or the 5′ terminal region of a functional entity, e.g. the transcribed region, of certain genes. The sequence starts with a cytidine, which usually corresponds to the transcriptional start site, and is followed by a stretch of usually about 3 to 30 pyrimidine nucleotides. For example, the TOP may comprise 3, 4, 5, 6, 7, 8, 9, 10, 11, 12, 13, 14, 15, 16, 17, 18, 19, 20, 21, 22, 23, 24, 25, 26, 27, 28, 29, 30 or even more nucleotides. The pyrimidine stretch and thus the 5′-TOP ends one nucleotide 5′ to the first purine nucleotide located downstream of the TOP. Messenger RNA that contains a 5′ terminal oligopyrimidine tract is often referred to as TOP mRNA. Accordingly, genes that provide such messenger RNAs are referred to as TOP genes. The term “TOP motif” or “5′-TOP motif” has to be understood as a nucleic acid sequence which corresponds to a 5′-TOP as defined above. Thus, a TOP motif in the context of the present invention is preferably a stretch of pyrimidine nucleotides having a length of 3-30 nucleotides. Preferably, the TOP-motif consists of at least 3 pyrimidine nucleotides, preferably at least 4 pyrimidine nucleotides, preferably at least 5 pyrimidine nucleotides, more preferably at least 6 nucleotides, more preferably at least 7 nucleotides, most preferably at least 8 pyrimidine nucleotides, wherein the stretch of pyrimidine nucleotides preferably starts at its 5′-end with a cytosine nucleotide. In TOP genes and TOP mRNAs, the TOP-motif preferably starts at its 5′-end with the transcriptional start site and ends one nucleotide 5′ to the first purine residue in said gene or mRNA. A TOP motif in the sense of the present invention is preferably located at the 5′-end of a sequence which represents a 5′-UTR or at the 5′-end of a sequence which codes for a 5′-UTR. Thus, preferably, a stretch of 3 or more pyrimidine nucleotides is called “TOP motif” in the sense of the present invention if this stretch is located at the 5′-end of a respective sequence, such as the artificial nucleic acid, the 5′-UTR element of the artificial nucleic acid, or the nucleic acid sequence which is derived from the 5′-UTR of a TOP gene as described herein. In other words, a stretch of 3 or more pyrimidine nucleotides, which is not located at the 5′-end of a 5′-UTR or a 5′-UTR element but anywhere within a 5′-UTR or a 5′-UTR element, is preferably not referred to as “TOP motif”. In some embodiments, the nucleic acid sequence of the 5′-UTR element, which is derived from a 5′-UTR of a TOP gene, terminates at its 3′-end with a nucleotide located at position 1, 2, 3, 4, 5, 6, 7, 8, 9 or 10 upstream of the start codon (e.g. A(U/T)G) of the gene or RNA it is derived from. Thus, the 5′-UTR element does not comprise any part of the protein coding sequence. Thus, preferably, the only protein coding part of the at least one nucleic acid sequence, particularly of the RNA sequence, is provided by the coding sequence.

### Short Description of the Invention

The present invention is based on the inventor's surprising finding that at least one peptide or protein derived from of a Yellow fever virus (YFV) protein, preferably a YFV prME polyprotein or a non-structural NS1 protein encoded by the artificial RNA of the invention can efficiently be expressed in a mammalian cell. Unexpectedly, the expression of the YFV proteins (prME polyprotein or NS1 protein) encoded by the artificial nucleic RNA could be increased in vitro and in vivo by selecting suitable heterologous 5′ untranslated regions (UTRs) and suitable heterologous 3′ untranslated regions (UTRs). Advantageously, said artificial RNA of the invention comprising advantageous 3′-UTR/5′-UTR combinations induce very efficient antigen-specific immune responses against the encoded YFV prME polyprotein. Said artificial RNA comprised in lipid nanoparticles (LNPs) very efficiently induces antigen-specific immune responses against YFV prME or NS1 at a very low dosages and dosing regimen. Accordingly, the artificial RNA of the invention is suitable for eliciting an immune response against YFV prME or NS1 in a mammalian subject, in particular, in a human subject. The artificial RNA of the invention is therefore suitable for use as a vaccine, e.g. as a veterinary vaccine, preferably as a human vaccine.

In a first aspect, the present invention provides an artificial nucleic acid, preferably an artificial RNA comprising at least one 5′ untranslated region (UTR) and/or at least one 3′ untranslated region (UTR); and at least one coding sequence operably linked to said 3′-UTR and/or 5′-UTR encoding at least one antigenic peptide or protein derived from a yellow fever virus peptide or protein, preferably a yellow fever virus prME polyprotein or a fragment or variant thereof. Further, an artificial RNA comprising at least one 5′ untranslated region (UTR) and/or at least one 3′ untranslated region (UTR); and at least one coding sequence operably linked to said 3′-UTR and/or 5′-UTR encoding at least one antigenic peptide or protein derived from a yellow fever virus NS1 protein or a fragment or variant thereof.

In preferred embodiments, the artificial RNA comprises at least one nucleic acid sequence derived from a 3′-UTR of a gene selected from an ALB7 gene, an alpha-globin gene, a PSMB3 gene, a CASP1 gene, a COX6B1 gene, a NDUFA1 gene and a, or from a homolog, a fragment or a variant thereof.

In preferred embodiments, the artificial RNA comprises at least one nucleic acid sequence derived from a 5′-UTR of gene selected from a RPL32 gene, a, a HSD17B4 gene, a ATP5A1 gene, a NDUFA4 gene, a NOSIP gene, a RPL31 gene, a SLC7A3 gene, or from a homolog, a fragment or variant of any one of these genes.

Suitably, the artificial RNA of the invention comprises at least one coding sequence encoding at least one antigenic peptide or protein derived from a Yellow fever virus prME polyprotein operably linked to a 3′-UTR and a 5′-UTR selected from a-1 (HSD17B4/PSMB3), a-2 (Ndufa4/PSMB3), a-4 (NDUFA4/PSMB3), b-4 (HSD17D4/CASP1), c-5 (ATP5A1/PSMB3), d-1 (RpI31/PSMB3), d-5 (Slc7a3/Ndufa1), g-4 (NOSIP/CASP1), h-4 (Slc7a3/CASP1), i-2 (RPL32/ALB7), or i-3 (α-globin gene), wherein a-1 (HSD17B4/PSMB3), a-4 (NDUFA4/PSMB3), b-4 (HSD17B4/CASP1), d-5 (Slc7a3/Ndufa1), c-5 (ATP5A1/PSMB3), i-3 (alpha-globin), or g-4 (NOSIP/CASP1) are preferred, and a-1 (HSD17B4/PSMB3), a-4 (NDUFA4/PSMB3), b-4 (HSD17B4/CASP1), c-5 (ATP5A1/PSMB3), or g-4 (NOSIP/CASP1) are particularly preferred. Further, the artificial RNA of the invention may comprise at least one coding sequence encoding at least one antigenic peptide or protein derived from a Yellow fever virus NS1 protein operably linked to a 3′-UTR and a 5′-UTR selected from a-1 (HSD17B4/PSMB3), a-2 (Ndufa4/PSMB3), a-4 (NDUFA4/PSMB3), b-4 (HSD17B4/CASP1), c-5 (ATP5A1/PSMB3), d-1 (RpI31/PSMB3), d-5 (Slc7a3/Ndufa1), g-4 (NOSIP/CASP1), h-4 (Slc7a3/CASP1), i-2 (RPL32/ALB7), or i-3 (α-globin gene), wherein a-1 (HSD17B4/PSMB3), a-4 (NDUFA4/PSMB3), b-4 (HSD17B4/CASP1), d-5 (Slc7a3/Ndufa1), c-5 (ATP5A1/PSMB3), i-3 (alpha-globin), or g-4 (NOSIP/CASP1) are preferred, and a-1 (HSD17B4/PSMB3), a-4 (NDUFA4/PSMB3), b-4 (HSD17B4/CASP1), c-5 (ATP5A1/PSMB3), or g-4 (NOSIP/CASP1) are particularly preferred.

The at least one antigenic peptide or protein derived from YFV may be an NS1 protein.

The at least one antigenic peptide or protein derived from YFV may be prME polyprotein or prME polyprotein additionally comprising a C-terminal overhang comprising a fragment of YFV non-structural protein NS1 and/or an N-terminal overhang comprising a fragment of YFV capsid protein C.

The artificial RNA may additionally encode a heterologous secretory signal peptide, wherein secretory signal peptides derived from IgE and JEV are preferred.

The artificial RNA may additionally encode a further virus element, wherein a JEV-Stem is preferred.

The artificial RNA may comprise a codon modified coding sequence selected from C maximized coding sequence, CAI maximized coding sequence, human codon usage adapted coding sequence, G/C content modified coding sequence, and G/C optimized coding sequence, or any combination thereof.

The artificial RNA may be an mRNA, a viral RNA, self-replicating RNA, a circular RNA, or a replicon RNA. In preferred embodiments, the artificial RNA is an mRNA.

The artificial RNA, preferably mRNA, may further comprise at least one selected from a cap structure, a poly(A)sequence, a poly(C)sequence, a histone-stem loop, a 3′-terminal sequence element.

In a second aspect, the present invention provides a composition comprising said artificial RNA.

The composition of the second aspect may comprise at least one artificial RNA of the invention encoding YF prME and at least one further artificial RNA encoding YF NS1.

The composition may comprise at least one artificial RNA of the invention complexed or at least partially complexed with one or more cationic or polycationic compound, preferably with a cationic or polycationic polymer, cationic or polycationic polysaccharide, cationic or polycationic lipid, cationic or polycationic protein, cationic or polycationic peptide, or any combinations thereof.

The artificial RNA comprised in the composition may be at least partially complexed with protamine.

The artificial RNA of the invention comprised in the composition may be complexed or associated with a polyethylene glycol/peptide polymer and a lipid component, preferably a lipidoid component.

Suitably, the composition may comprise the artificial RNA of the invention complexed with, encapsulated in, or associated with one or more lipids, thereby forming lipid nanoparticles.

The composition may preferably comprise the artificial RNA of the invention complexed with one or more lipids thereby forming lipid nanoparticles (LNP), wherein the LNP essentially consists of

(i) at least one cationic lipid as defined herein, preferably a lipid of Formula (III), more preferably lipid III-3;
(ii) a neutral lipid as defined herein, preferably 1,2-distearoyl-sn-glycero-3-phosphocholine (DSPC);
(iii) a steroid or steroid analogue as defined herein, preferably cholesterol; and
(iv) a PEG-lipid as defined herein, e.g. PEG-DMG or PEG-cDMA, preferably a PEGylated lipid of Formula (IVa);
wherein (i) to (iv) are in a molar ratio of about 20-60% cationic lipid:5-25% neutral lipid:25-55% sterol; 0.5-15% PEG-lipid.

The present invention also concerns a yellow fever virus vaccine comprising said artificial RNA or said composition.

The present invention is also directed to the use of the artificial RNA, the composition and the vaccine in treatment or prophylaxis of an infection with a yellow fever virus.

In particular, the present invention is directed to the use of the artificial RNA, the composition and the vaccine in treatment or prophylaxis of an infection with yellow fever virus or a disorder related to such an infection.

The invention further concerns a method of treating or preventing a disorder or a disease in a subject, first and second medical uses of the artificial RNA, compositions and vaccines. Further, the invention is directed to a kit, particularly to a kit of parts, comprising the artificial RNA, compositions and vaccines.

### DETAILED DESCRIPTION OF THE INVENTION

The present application is filed together with a sequence listing in electronic format, which is part of the description of the present application (WIPO standard ST.25). The information contained in the electronic format of the sequence listing filed together with this application is incorporated herein by reference in its entirety. Where reference is made herein to a “SEQ ID NO:” the corresponding nucleic acid sequence or amino acid (aa) sequence in the sequence listing having the respective identifier is referred to. For many sequences, the sequence listing also provides additional detailed information, e.g. regarding certain structural features, sequence optimizations, GenBank identifiers, or regarding its coding capacity. In particular, such information is provided under numeric identifier <223> in the WIPO standard ST.25 sequence listing. Accordingly, information provided under said numeric identifier <223> is explicitly included herein in its entirety and has to be understood as integral part of the description of the underlying invention.

### Artificial Nucleic Acid:

In a first aspect, the invention relates to an artificial nucleic acid comprising

a) at least one 5′ untranslated region (UTR) and/or at least one 3′ untranslated region (UTR); and
b) at least one coding sequence operably linked to said 3′-UTR and/or 5′-UTR encoding at least one antigenic peptide or protein derived from a Yellow fever virus (YFV) or a fragment or variant thereof.

In a preferred embodiment of the first aspect, the invention relates to an artificial RNA, preferably an RNA suitable for vaccination, comprising

a) at least one heterologous 5′ untranslated region (5′-UTR) and/or at least one heterologous 3′ untranslated region (3′-UTR); and
b) at least one coding sequence operably linked to said 3′-UTR and/or 5′-UTR encoding at least one antigenic peptide or protein derived from a YFV prME polyprotein or a fragment or variant thereof.

In other preferred embodiment of the first aspect, the invention relates to an artificial RNA, preferably an RNA suitable for vaccination, comprising

a) at least one heterologous 5′ untranslated region (5′-UTR) and/or at least one heterologous 3′ untranslated region (3′-UTR); and
b) at least one coding sequence operably linked to said 3′-UTR and/or 5′-UTR encoding at least one antigenic peptide or protein derived from a YFV NS1 protein or a fragment or variant thereof.

In general, RNA is composed of a protein-coding region, and 5′- and 3′-untranslated regions (UTRs). The 3′-UTR is variable in sequence and size; it spans between the stop codon and the poly(A) tail. Importantly, the 3′-UTR sequence harbors several regulatory motifs that determine RNA turnover, stability and localization, and thus governs many aspects of post-transcriptional regulation. In medical application of nucleic acids, e.g. RNA (e.g. immunotherapy applications, vaccination) the regulation of nucleic acid translation into protein is of paramount importance to therapeutic safety and efficacy. The present inventors surprisingly discovered that certain combinations of 3′-UTRs and 5′-UTRs act in concert to synergistically enhance the expression of operably linked nucleic acid sequences encoding YFV antigenic peptides or proteins. Artificial RNA molecules harboring the inventive UTR combinations advantageously enable the rapid and transient expression of high amounts of YFV antigenic peptides or proteins. Accordingly, the artificial RNA provided herein is particularly useful and suitable for various applications in vivo, including the vaccination against YFV.

Suitably, the artificial RNA may comprise at least one heterologous 5′-UTR and/or at least one heterologous 3′-UTR. In this context, an UTR of the invention comprises or consists of a nucleic acid sequence derived from a 5′-UTR or a 3′-UTR of any naturally occurring gene or a fragment, a homolog or a variant thereof. Preferably, a 5′-UTR or a 3′-UTR of the invention is heterologous to the at least one coding sequence encoding the at least one antigenic peptide or protein derived from a Yellow fever virus. Suitable heterologous 5′-UTRs or heterologous 3′-UTRs are derived from naturally occurring genes (that are not derived from YFV). In other embodiments, synthetically engineered 5′-UTRs or 3′-UTRs may be used in the context of the present invention.

In preferred embodiments, the at least one artificial RNA comprises at least one heterologous 3′-UTR.

Preferably, the at least one heterologous 3′-UTR comprises or consists of a nucleic acid sequence derived from a 3′-UTR of a chordate gene, preferably a vertebrate gene, more preferably a mammalian gene, most preferably a human gene, or from a variant of a 3′-UTR of a chordate gene, preferably a vertebrate gene, more preferably a mammalian gene, most preferably a human gene.

Preferably the artificial RNA of the present invention comprises a 3′-UTR, which may be derivable from a gene that relates to an RNA with an enhanced half-life (that provides a stable RNA), for example a 3′-UTR as defined and described below.

Preferably, the at least one heterologous 3′-UTR comprises a nucleic acid sequence derived from a 3′-UTR of a gene, which preferably encodes a stable mRNA, or from a homolog, a fragment or a variant of said gene.

In preferred embodiments of the first aspect, the artificial RNA of the invention comprises at least one heterologous 3′-UTR, wherein the at least one heterologous 3′-UTR comprises a nucleic acid sequence derived from a 3′-UTR of a gene selected from an ALB7 gene, an alpha-globin gene, a PSMB3 gene, a CASP1 gene, a COX6B1 gene, a NDUFA1 gene, or from a homolog, a fragment or a variant thereof.

### ALB7-Derived 3′-UTR:

In preferred embodiments, the 3′-UTR comprises or consists of a nucleic acid sequence which is derived from the 3′-UTR of a vertebrate albumin gene or from a variant thereof, preferably from the 3′-UTR of a mammalian albumin gene or from a variant thereof, more preferably from the 3′-UTR of a human albumin gene or from a variant thereof, even more preferably from the 3′-UTR of the human albumin gene according to GenBank Accession number NM_000477.5, or from a homolog, fragment or variant thereof.

Accordingly, the artificial RNA of the invention may comprise a 3′-UTR derived from a ALB7 gene, wherein said 3′-UTR derived from a ALB7 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 23, 24 or a fragment or a variant thereof.

### Alpha-Globin Gene-Derived 3′-UTR:

In preferred embodiments, the 3′-UTR comprises or consists of a nucleic acid sequence which is derived from the 3′-UTR of a vertebrate alpha-globin gene (herein referred to as “muag”) or from a variant thereof, preferably from the 3′-UTR of a mammalian alpha-globin or from a variant thereof, more preferably from the 3′-UTR of a human alpha-globin gene or from a variant thereof, even more preferably from the 3′-UTR of the human alpha-globin gene.

Accordingly, the RNA of the invention may comprise a 3′-UTR derived from a alpha-globin gene, wherein said 3′-UTR derived from a alpha-globin gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 25, 26 or a fragment or a variant thereof.

### PSMB3-Derived 3′-UTR:

The artificial RNA of the invention may comprise a 3′-UTR which is derived from a 3′-UTR of a gene encoding a proteasome subunit beta type-3 (PSMB3) protein, or a homolog, variant, fragment or derivative thereof. Such 3′-UTRs preferably comprise or consist of a nucleic acid sequences derived from the 3′-UTR of a proteasome subunit beta type-3 (PSMB3) gene, preferably from a vertebrate, more preferably a mammalian, most preferably a human proteasome subunit beta type-3 (PSMB3) gene, or a homolog, variant, fragment or derivative thereof. Said gene may preferably encode a proteasome subunit beta type-3 (PSMB3) protein corresponding to a human proteasome subunit beta type-3 (PSMB3) protein (UniProt Ref. No. P49720, entry version #183 of 30 Aug. 2017).

Accordingly, the artificial RNA of the invention may comprise a 3′-UTR derived from a PSMB3 gene, wherein said 3′-UTR derived from a PSMB3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 15, 16 or a fragment or a variant thereof.

### CASP1-Derived 3′-UTR:

The artificial RNA of the invention may comprise a 3′-UTR which is derived from a 3′-UTR of a gene encoding a Caspase-1 (CASP1) protein, or a homolog, variant, fragment or derivative thereof. Such 3′-UTRs preferably comprise or consist of a nucleic acid sequence derived from the 3′-UTR of a Caspase-1 (CASP1) gene, preferably from a vertebrate, more preferably a mammalian, most preferably a human Caspase-1 (CASP1) gene, or a homolog, variant, fragment or derivative thereof.

Accordingly, the RNA of the invention may comprise a 3′-UTR derived from a CASP1 gene, wherein said 3′-UTR derived from a CASP1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 17, 18 or a fragment or a variant thereof.

### COX6B1-Derived 3′-UTR:

The artificial RNA of the invention may comprise a 3′-UTR which is derived from a 3′-UTR of a COX6B1 gene encoding a cytochrome c oxidase subunit 6B1 (COX6B1) protein, or a homolog, variant, fragment or derivative thereof. Such 3′-UTRs preferably comprise or consist of a nucleic acid sequence which is derived from the 3′-UTR of a cytochrome c oxidase subunit 6B1 (COX6B1) gene, preferably from a vertebrate, more preferably a mammalian, most preferably a human cytochrome c oxidase subunit 6B1 (COX6B1) gene, or a homolog, variant, fragment or derivative thereof. Said gene may preferably encode a cytochrome c oxidase subunit 6B1 (COX6B1) protein corresponding to a human cytochrome c oxidase subunit 6B1 (COX6B1) protein (UniProt Ref. No. P14854, entry version #166 of 30 Aug. 2017).

Accordingly, the artificial RNA of the invention may comprise a 3′-UTR derived from a COX6B1 gene, wherein said 3′-UTR derived from a COX6B1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 21, 22 or a fragment or a variant thereof.

### NDUFA1-Derived 3′-UTR:

The artificial RNA of the invention may comprise a 3′-UTR which is derived from a 3′-UTR of a gene encoding a NADH dehydrogenase [ubiquinone] 1 alpha sub complex subunit 1 (NDUFA1) protein, or a homolog, variant, fragment or derivative thereof. Such 3′-UTRs preferably comprise or consist of a nucleic acid sequence derived from the 3′-UTR of a NADH dehydrogenase [ubiquinone] 1 alpha sub complex subunit 1 (NDUFA1) gene, preferably from a vertebrate, more preferably a mammalian NADH dehydrogenase [ubiquinone] 1 alpha sub complex subunit 1 (NDUFA1) gene, or a homolog, variant, fragment or derivative thereof. Said gene may preferably encode a NADH dehydrogenase [ubiquinone] 1 alpha sub complex subunit 1 (NDUFA1) protein corresponding to a human NADH dehydrogenase [ubiquinone] 1 alpha sub complex subunit 1 (NDUFA1) protein (UniProt Ref. No. 015239, entry version #152 of 30 Aug. 2017).

Accordingly, the artificial RNA of the invention may comprise a 3′-UTR derived from a NDUFA1 gene, wherein said 3′-UTR derived from a NDUFA1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 19, 20 or a fragment or a variant thereof.

In embodiments, the artificial RNA as defined herein comprises a 3′-UTR as described in WO2016/107877. In this context, the disclosure of WO2016/107877 relating to 3′-UTR sequences is herewith incorporated by reference. Particularly suitable 3′-UTRs are SEQ ID NOs: 1-24 and SEQ ID NOs: 49-318 of patent application WO2016/107877, or fragments or variants of these sequences. Accordingly, the 3′-UTRs of the artificial RNA of the present invention may comprise or consists of a corresponding RNA sequence of the nucleic acid sequence according SEQ ID NOs: 1-24 and SEQ ID NOs: 49-318 of the patent application WO2016/107877.

In embodiments, the artificial RNA as defined herein comprises a 3′-UTR as described in WO2017/036580. In this context, the disclosure of WO2017/036580 relating to 3′-UTR sequences is herewith incorporated by reference. Particularly suitable 3′-UTRs are SEQ ID NOs: 152-204 of the patent application WO2017/036580, or fragments or variants of these sequences. Accordingly, the 3′-UTR of the artificial RNA of the present invention may comprise or consist of a corresponding RNA sequence of the nucleic acid sequence according SEQ ID NOs: 152-204 of the patent application WO2017/036580.

According to preferred embodiments the artificial RNA comprises at least one heterologous 5′-UTR.

In preferred embodiments, the at least one artificial nucleic acid as defined herein, particularly the RNA as defined herein may comprise at least one heterologous 5′-UTR.

Preferably, the at least one 5′-UTR comprises or consists of a nucleic acid sequence derived from the 5′-UTR of a chordate gene, preferably a vertebrate gene, more preferably a mammalian gene, most preferably a human gene, or from a variant of the 5′-UTR of a chordate gene, preferably a vertebrate gene, more preferably a mammalian gene, most preferably a human gene.

Preferably the artificial RNA of the present invention comprises a 5′-UTR, which may be derivable from a gene that relates to an RNA with an enhanced half-life (that provides a stable RNA), for example a 5′-UTR as defined and described below.

Preferably, the at least one heterologous 5′-UTR comprises a nucleic acid sequence derived from a 5′-UTR of a gene, which preferably encodes a stable mRNA, or from a homolog, a fragment or a variant of said gene.

In preferred embodiments of the first aspect, the artificial RNA of the invention comprises at least one heterologous 5′-UTR, wherein the at least one heterologous 5′-UTR comprises a nucleic acid sequence derived from a 5′-UTR of gene selected from a RPL32 gene, a HSD17B4 gene, a ATP5A1 gene, a NDUFA4 gene, a NOSIP gene, a RPL31 gene, a SLC7A3 gene, or from a homolog, a fragment or variant of any one of these genes.

### RPL32-Derived 5′-UTR:

The artificial RNA of the invention may comprise a 5′-UTR derived from a 5′-UTR of a gene encoding a 60S ribosomal protein L32, or a homolog, variant, fragment or derivative thereof, wherein said 5′-UTR preferably lacks the 5′TOP motif. Such 5′-UTRs preferably comprise or consist of a nucleic acid sequence derived from the 5′-UTR of a 60S ribosomal protein L32 (RPL32) gene, preferably from a vertebrate, more preferably a mammalian, most preferably a human 60S ribosomal protein L32 (RPL32) gene, or a homolog, variant, fragment or derivative thereof, wherein the 5′-UTR preferably does not comprise the 5′TOP of said gene. Said gene may preferably encode a 60S ribosomal protein L32 (RPL32) corresponding to a human 60S ribosomal protein L32 (RPL32) (UniProt Ref. No. P62899, entry version #138 of 30 Aug. 2017).

Accordingly, the artificial RNA of the invention may comprise a 5′-UTR derived from a RPL32 gene, wherein said 5′-UTR derived from a RPL32 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 13, 14 or a fragment or a variant thereof.

### HSD17B4-Derived 5′-UTR:

The artificial RNA of the invention may comprise a 5′-UTR derived from a 5′-UTR of a gene encoding a 17-beta-hydroxysteroid dehydrogenase 4, or a homolog, variant, fragment or derivative thereof, preferably lacking the 5′TOP motif. Such 5′-UTRs preferably comprise or consist of a nucleic acid sequence derived from the 5′-UTR of a 17-beta-hydroxysteroid dehydrogenase 4 (also referred to as peroxisomal multifunctional enzyme type 2) gene, preferably from a vertebrate, more preferably mammalian, most preferably human 17-beta-hydroxysteroid dehydrogenase 4 (HSD17B4) gene, or a homolog, variant, fragment or derivative thereof, wherein preferably the 5′-UTR does not comprise the 5′TOP of said gene. Said gene may preferably encode a 17-beta-hydroxysteroid dehydrogenase 4 protein corresponding to human 17-beta-hydroxysteroid dehydrogenase 4 (UniProt Ref. No. Q9BPX1, entry version #139 of Aug. 30, 2017), or a homolog, variant, fragment or derivative thereof.

Accordingly, the artificial RNA of the invention may comprise a 5′-UTR derived from a HSD17B4 gene, wherein said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1, 2 or a fragment or a variant thereof.

### ATP5A1-Derived 5′-UTR:

The artificial RNA of the invention may comprise a 5′-UTR which is derived from a 5′-UTR of a gene encoding mitochondrial ATP synthase subunit alpha (ATP5A1), or a homolog, variant, fragment or derivative thereof, wherein said 5′-UTR preferably lacks the 5′TOP motif. Such 5′-UTRs preferably comprise or consist of a nucleic acid sequence derived from the 5′-UTR of a mitochondrial ATP synthase subunit alpha (ATP5A1) gene, preferably from a vertebrate, more preferably a mammalian and most preferably a human mitochondrial ATP synthase subunit alpha (ATP5A1) gene, or a homolog, variant, fragment or derivative thereof, wherein the 5′-UTR preferably does not comprise the 5′TOP of said gene. Said gene may preferably encode a mitochondrial ATP synthase subunit alpha protein corresponding to human acid mitochondrial ATP synthase subunit alpha (UniProt Ref. No. P25705, entry version #208 of Aug. 30, 2017), or a homolog, variant, fragment or derivative thereof.

Accordingly, the artificial RNA of the invention may comprise a 5′-UTR derived from a ATP5A1 gene, wherein said 5′-UTR derived from a ATP5A1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 5, 6 or a fragment or a variant thereof.

### NDUFA4-Derived 5′-UTR:

The artificial RNA of the invention may comprise a 5′-UTR which is derived from a 5′-UTR of a gene encoding a Cytochrome c oxidase subunit (NDUFA4), or a homolog, fragment or variant thereof. Such 5′-UTRs preferably comprise or consist of a nucleic acid sequence derived from the 5′-UTR of a Cytochrome c oxidase subunit (NDUFA4) gene, preferably from a vertebrate, more preferably a mammalian Cytochrome c oxidase subunit (NDUFA4) gene, or a homolog, variant, fragment or derivative thereof. Said gene may preferably encode a Cytochrome c oxidase subunit (NDUFA4) protein corresponding to a human Cytochrome c oxidase subunit (NDUFA4) protein (UniProt Ref. No. 000483, entry version #149 of 30 Aug. 2017).

Accordingly, the artificial RNA of the invention may comprise a 5′-UTR derived from a NDUFA4 gene, wherein said 5′-UTR derived from a NDUFA4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 3, 4 or a fragment or a variant thereof.

### NOSIP-Derived 5′-UTR:

The artificial RNA of the invention may comprise a 5′-UTR which is derived from a 5′-UTR of a gene encoding a Nitric oxide synthase-interacting (NOSIP) protein, or a homolog, variant, fragment or derivative thereof. Such 5′-UTRs preferably comprise or consist of a nucleic acid sequence derived from the 5′-UTR of a Nitric oxide synthase-interacting protein (NOSIP) gene, preferably from a vertebrate, more preferably a mammalian, most preferably a human Nitric oxide synthase-interacting protein (NOSIP) gene, or a homolog, variant, fragment or derivative thereof. Said gene may preferably encode a Nitric oxide synthase-interacting protein (NOSIP) protein corresponding to a human Nitric oxide synthase-interacting protein (NOSIP) protein (UniProt Ref. No. Q9Y314, entry version #130 of 7 Jun. 2017).

Accordingly, the artificial RNA of the invention may comprise a 5′-UTR derived from a NOSIP gene, wherein said 5′-UTR derived from a NOSIP gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 7, 8 or a fragment or a variant thereof.

RPL31-derived 5′-UTR:

The artificial RNA of the invention may comprise a 5′-UTR which is derived from a 5′-UTR of a gene encoding a 60S ribosomal protein L31, or a homolog, variant, fragment or derivative thereof, wherein said 5′-UTR preferably lacks the 5′TOP motif. Such 5′-UTR preferably comprise or consist of a nucleic acid sequence derived from the 5′-UTR of a 60S ribosomal protein L31 (RPL31) gene, preferably from a vertebrate, more preferably a mammalian, most preferably a human 60S ribosomal protein L31 (RPL31) gene, or a homolog, variant, fragment or derivative thereof, wherein the 5′-UTR preferably does not comprise the 5′TOP of said gene. Said gene may preferably encode a 60S ribosomal protein L31 (RPL31) corresponding to a human 60S ribosomal protein L31 (RPL31) (UniProt Ref. No. P62899, entry version #138 of 30 Aug. 2017).

Accordingly, the artificial RNA of the invention may comprise a 5′-UTR derived from a RPL31 gene, wherein said 5′-UTR derived from a RPL31 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 9, 10 or a fragment or a variant thereof.

### SLC7A3-Derived 5′-UTR:

The artificial RNA of the invention may comprise a 5′-UTR which is derived from a 5′-UTR of a gene encoding a cationic amino acid transporter 3 (solute carrier family 7 member 3, SLC7A3) protein, or a homolog, variant, fragment or derivative thereof. Such 5′-UTRs preferably comprise or consist of a nucleic acid sequence derived from the 5′-UTR of a cationic amino acid transporter 3 (SLC7A3) gene, preferably from a vertebrate, more preferably a mammalian cationic amino acid transporter 3 (SLC7A3) gene, or a homolog, variant, fragment or derivative thereof. Said gene may preferably encode a cationic amino acid transporter 3 (SLC7A3) protein corresponding to a human cationic amino acid transporter 3 (SLC7A3) protein (UniProt Ref. No. Q8WY07, entry version #139 of 30 Aug. 2017).

Accordingly, the artificial RNA of the invention may comprise a 5′-UTR derived from a SLC7A3 gene, wherein said 5′-UTR derived from a SLC7A3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 11, 12 or a fragment or a variant thereof.

In embodiments, the artificial RNA as defined herein comprises a 5′-UTR as described in WO2013/143700. In this context, the disclosure of WO2013/143700 relating to 5′-UTR sequences is herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of the patent application WO2013/143700, or fragments or variants of these sequences. In this context, it is preferred that the 5′-UTR of the artificial RNA according to the present invention comprises or consists of a corresponding RNA sequence of the nucleic acid sequence according SEQ ID NOs: 1-1363, SEQ ID NO: 1395, SEQ ID NO: 1421 and SEQ ID NO: 1422 of the patent application WO2013/143700.

In embodiments, the artificial RNA of the invention comprises a 5′-UTR as described in WO2016/107877. In this context, the disclosure of WO2016/107877 relating to 5′-UTR sequences is herewith incorporated by reference. Particularly preferred 5′-UTRs are nucleic acid sequences according to SEQ ID NOs: 25-30 and SEQ ID NOs: 319-382 of the patent application WO2016/107877, or fragments or variants of these sequences. In this context, it is particularly preferred that the 5′-UTR of the artificial RNA comprises or consists of a corresponding RNA sequence of the nucleic acid sequence according SEQ ID NOs: 25-30 and SEQ ID NOs: 319-382 of the patent application WO2016/107877.

In embodiments, the artificial RNA of the invention comprises a 5′-UTR as described in WO2017/036580. In this context, the disclosure of WO2017/036580 relating to 5′-UTR sequences is herewith incorporated by reference. Particularly preferred 5′-UTRs are nucleic acid sequences according to SEQ ID NOs: 1-151 of the patent application WO2017/036580, or fragments or variants of these sequences. In this context, it is particularly preferred that the 5′-UTR of the artificial RNA comprises or consists of a corresponding RNA sequence of the nucleic acid sequence according to SEQ ID NOs: 1-151 of the patent application WO2017/036580.

The inventors observed that certain combinations of at least one heterologous 5′-UTR and/or at least one heterologous 3′-UTR are advantageously increasing the translation of the at least one coding sequence operably linked to said 3′-UTR and/or 5′-UTR encoding at least one antigenic peptide or protein derived from a YFV prME polyprotein or an YFV NS1 protein.

Accordingly it is preferred that the at least one heterologous 5′-UTR as defined herein and the at least one heterologous 3′-UTR as defined herein act synergistically to increase production of antigenic peptide or protein from the artificial RNA of the invention. These advantageous combinations of 5′-UTR and 3′ UTR are specified in the following.

In preferred embodiments, the artificial RNA of the invention comprises at least one coding sequence encoding at least one antigenic peptide or protein derived from a YFV prME polyprotein operably linked to a 5′-UTR of a HSD17B4 gene and a 3′-UTR of a PSMB3 gene (UTR combination herein referred to as “a-1”)

In preferred embodiments, the artificial RNA of the invention comprises at least one coding sequence encoding at least one antigenic peptide or protein derived from a YFV prME polyprotein operably linked to a 5′-UTR of a Ndufa4 gene and a 3′-UTR of a PSMB3 gene (UTR combination herein referred to as “a-2”).

In preferred embodiments, the artificial RNA of the invention comprises at least one coding sequence encoding at least one antigenic peptide or protein derived from a YFV prME polyprotein operably linked to a 5′-UTR of a NOSIP gene and a 3′-UTR of a PSMB3 gene (UTR combination herein referred to as “a-4”).

In preferred embodiments, the artificial RNA of the invention comprises at least one coding sequence encoding at least one antigenic peptide or protein derived from a YFV prME polyprotein operably linked to a 5′-UTR of a HSD17B4 gene and a 3′-UTR of a CASP1 gene (UTR combination herein referred to as “b-4”).

In preferred embodiments, the artificial RNA of the invention comprises at least one coding sequence encoding at least one antigenic peptide or protein derived from a YFV prME polyprotein operably linked to a 5′-UTR of a ATP5A1 gene and a 3′-UTR of a PSMB3 gene (UTR combination herein referred to as “c-5”).

In preferred embodiments, the artificial RNA of the invention comprises at least one coding sequence encoding at least one antigenic peptide or protein derived from a YFV prME polyprotein operably linked to a 5′-UTR of a RPL31 gene and a 3′-UTR of a PSMB3 gene (UTR combination herein referred to as “d-1”).

In preferred embodiments, the artificial RNA of the invention comprises at least one coding sequence encoding at least one antigenic peptide or protein derived from a YFV prME polyprotein operably linked to a 5′-UTR of a SLC7A3 gene and a 3′-UTR of a NDUFA1 gene (UTR combination herein referred to as “d-5”).

In preferred embodiments, the artificial RNA of the invention comprises at least one coding sequence encoding at least one antigenic peptide or protein derived from a YFV prME polyprotein operably linked to a 5′-UTR of a NOSIP gene and a 3′-UTR of a CASP1 gene (UTR combination herein referred to as “g-4”).

In preferred embodiments, the artificial RNA of the invention comprises at least one coding sequence encoding at least one antigenic peptide or protein derived from a YFV prME polyprotein operably linked to a 5′-UTR of a SLC7A3 gene and 3′-UTR of a CASP1 gene (UTR combination herein referred to as “h-4”).

In preferred embodiments, the artificial RNA of the invention comprises at least one coding sequence encoding at least one antigenic peptide or protein derived from a YFV prME polyprotein operably linked to a 5′-UTR of a SLC7A3 gene and a 3′-UTR of a COX6B1 gene (UTR combination herein referred to as “h-5”).

In preferred embodiments, the artificial RNA of the invention comprises at least one coding sequence encoding at least one antigenic peptide or protein derived from a YFV prME polyprotein operably linked to a 5′-UTR of a RPL32 gene and a 3′-UTR of a ALB7 gene (UTR combination herein referred to as “i-2”).

In preferred embodiments, the artificial RNA of the invention comprises at least one coding sequence encoding at least one antigenic peptide or protein derived from a YFV prME polyprotein operably linked to a 3′-UTR of an alpha-globin gene (herein referred to as “i-3”).

In other preferred embodiments, the artificial RNA of the invention comprises at least one coding sequence encoding at least one antigenic peptide or protein derived from a YFV NS1 protein operably linked to a 5′-UTR of a HSD17B4 gene and a 3′-UTR of a PSMB3 gene (UTR combination herein referred to as “a-1”)

In other preferred embodiments, the artificial RNA of the invention comprises at least one coding sequence encoding at least one antigenic peptide or protein derived from a YFV NS1 protein operably linked to a 5′-UTR of a Ndufa4 gene and a 3′-UTR of a PSMB3 gene (UTR combination herein referred to as “a-2”).

In other preferred embodiments, the artificial RNA of the invention comprises at least one coding sequence encoding at least one antigenic peptide or protein derived from a YFV NS1 protein operably linked to a 5′-UTR of a NOSIP gene and a 3′-UTR of a PSMB3 gene (UTR combination herein referred to as “a-4”).

In other preferred embodiments, the artificial RNA of the invention comprises at least one coding sequence encoding at least one antigenic peptide or protein derived from a YFV NS1 protein operably linked to a 5′-UTR of a HSD17B4 gene and a 3′-UTR of a CASP1 gene (UTR combination herein referred to as “b-4”).

In other preferred embodiments, the artificial RNA of the invention comprises at least one coding sequence encoding at least one antigenic peptide or protein derived from a YFV NS1 protein operably linked to a 5′-UTR of a ATP5A1 gene and a 3′-UTR of a PSMB3 gene (UTR combination herein referred to as “c-5”).

In other preferred embodiments, the artificial RNA of the invention comprises at least one coding sequence encoding at least one antigenic peptide or protein derived from a YFV NS1 protein operably linked to a 5′-UTR of a RPL31 gene and a 3′-UTR of a PSMB3 gene (UTR combination herein referred to as “d-1”).

In other preferred embodiments, the artificial RNA of the invention comprises at least one coding sequence encoding at least one antigenic peptide or protein derived from a YFV NS1 protein operably linked to a 5′-UTR of a SLC7A3 gene and a 3′-UTR of a NDUFA1 gene (UTR combination herein referred to as “d-5”).

In other preferred embodiments, the artificial RNA of the invention comprises at least one coding sequence encoding at least one antigenic peptide or protein derived from a YFV NS1 protein operably linked to a 5′-UTR of a NOSIP gene and a 3′-UTR of a CASP1 gene (UTR combination herein referred to as “g-4”).

In other preferred embodiments, the artificial RNA of the invention comprises at least one coding sequence encoding at least one antigenic peptide or protein derived from a YFV NS1 protein operably linked to a 5′-UTR of a SLC7A3 gene and 3′-UTR of a CASP1 gene (UTR combination herein referred to as “h-4”).

In other preferred embodiments, the artificial RNA of the invention comprises at least one coding sequence encoding at least one antigenic peptide or protein derived from a YFV NS1 protein operably linked to a 5′-UTR of a SLC7A3 gene and a 3′-UTR of a COX6B1 gene (UTR combination herein referred to as “h-5”).

In other preferred embodiments, the artificial RNA of the invention comprises at least one coding sequence encoding at least one antigenic peptide or protein derived from a YFV NS1 protein operably linked to a 5′-UTR of a RPL32 gene and a 3′-UTR of a ALB7 gene (UTR combination herein referred to as “i-2”).

In other preferred embodiments, the artificial RNA of the invention comprises at least one coding sequence encoding at least one antigenic peptide or protein derived from a YFV NS1 protein operably linked to a 3′-UTR of an alpha-globin gene (herein referred to as “i-3”).

Accordingly, in preferred embodiments of the first aspect, the artificial RNA of the invention comprises

a-1. at least one 5′-UTR derived from a 5′-UTR of a HSD17B4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
a-2. at least one 5′-UTR derived from a 5′-UTR of a NDUFA4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
a-4. at least one 5′ UTR derived from a 5′UTR of a NOSIP gene, or from a corresponding RNA sequence, homolog, fragment or variant thereof and at least one 3′ UTR derived from a 3′UTR of a PSMB3 gene, or from a corresponding RNA sequence, homolog, fragment or variant thereof; or
b-4. at least one 5′-UTR derived from a 5′-UTR of a HSD17B4 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
c-5. at least one 5′-UTR derived from a 5′-UTR of a ATP5A1 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
d-1. at least one 5′-UTR derived from a 5′-UTR of a RPL31 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
d-5. at least one 5′-UTR derived from a 5′-UTR of a SLC7A3 gene, or from a corresponding RNA sequence, homolog, fragment or variant thereof and at least one 3′-UTR derived from a 3′-UTR of a NDUFA1 gene, or from a corresponding RNA sequence, homolog, fragment or variant thereof; or
g-4. at least one 5′-UTR element derived from a 5′-UTR of a NOSIP gene, or from a corresponding RNA sequence, homolog, fragment or variant thereof and at least one 3′-UTR element derived from a 3′-UTR of a CASP1 gene, or from a corresponding RNA sequence, homolog, fragment or variant thereof; or
h-4. at least one 5′-UTR derived from a 5′-UTR of a SLC7A3 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
h-5. at least one 5′-UTR derived from a 5′-UTR of a SLC7A3 gene, or from a corresponding RNA sequence, homolog, fragment or variant thereof and at least one 3′-UTR derived from a 3′-UTR of a COX6B1 gene, or from a corresponding RNA sequence, homolog, fragment or variant thereof; or
i-2. at least one 5′-UTR derived from a 5′-UTR of a RPL32 gene, or from a corresponding RNA sequence, homolog, fragment or variant thereof and at least one 3′-UTR derived from a 3′-UTR of a ALB7 gene, or from a corresponding RNA sequence, homolog, fragment or variant thereof.
i-3. at least one 3′-UTR derived from a 3′-UTR of a alpha-globin gene, or from a corresponding RNA sequence, homolog, fragment or variant thereof.

In particularly preferred embodiments of the first aspect, the artificial RNA of the invention comprises UTR elements according to a-1 (HSD17B4/PSMB3), a-4 (NDUFA4/PSMB3), b-4 (HSD17B4/CASP1), d-5 (Slc7a3/Ndufa1), c-5 (ATP5A1/PSMB3), i-3 (alpha-globin), or g-4 (NOSIP/CASP1).

In further, even more preferred embodiments of the first aspect, the artificial RNA of the invention comprises

UTR elements according to a-1 (HSD1764/PSMB3), a-4 (NDUFA4/PSMB3), b-4 (HSD17B4/CASP1), c-5 (ATP5A1/PSMB3), or g-4 (NOSIP/CASP1).

In a particularly preferred embodiment of the first aspect, the artificial RNA of the invention comprises UTR elements according to a-1 (HSD17B4/PSMB3).

The invention relates to an artificial RNA, preferably an RNA suitable for vaccination, comprising at least one heterologous 5′-UTR as defined above and/or at least one heterologous 3′-UTR as defined above and at least one coding sequence operably linked to said 3′-UTR and/or 5′-UTR, wherein said coding sequence encodes at least one antigenic peptide or protein derived from a Yellow fever virus, or a fragment or variant thereof.

As used herein, the term “yellow fever virus” or the corresponding abbreviation “YFV” is not limited to a particular virus strain, variant, serotype, or isolate, etc. but comprises any yellow fever virus of any origin.

According to preferred embodiments, the artificial RNA, preferably the coding sequence of the artificial RNA comprises or consists of a nucleic acid sequence that is derived from a YFV selected from yellow fever viruses listed in the following, with NCBI Taxonomy ID (“NCBI-ID”) and/or UniprotKB/Swiss Prot/Genbank ID (“GB-ID”) provided below: YFV 17D (NCBI-ID:11090; GB-ID:P03314), YFV 1899/81 (NCBI-ID:31641; GB-ID:P29165), YFV isolate Angola/14FA/1971 (NCBI-ID:407140; GB-ID:Q1X881), YFV isolate Ethiopia/Couma/1961 (NCBI-ID:407141; GB-ID:Q074N0), YFV isolate Ivory Coast/1999 (NCBI-ID:407136; GB-ID:Q6J3P1), YFV isolate Ivory Coast/85-82H/1982 (NCBI-ID:407138; GB-ID:Q6J3P1), YFV isolate Uganda/A7094A4/1948 (NCBI-ID:407139; GB-ID:Q1X880), YFV strain French neurotropic vaccine (NCBI-ID:407135; GB-ID:Q89277), YFV strain Ghana/Asibi/1927 (NCBI-ID:407134; GB-ID:Q6DV88), and YFV Trinidad/79A/1979 (NCBI-ID:407137; GB-ID:Q9YRV3).

The RNA genome of YFV typically encodes a plurality of structural and non-structural proteins. Translation of viral RNA typically leads to a precursor protein comprising a plurality of individual viral (structural and non-structural) proteins (or precursor of these proteins) in one polypeptide chain, which is typically referred to as “polyprotein” or “precursor protein”. The “polyprotein” or “precursor protein” or is processed by proteases into ten functionally distinct proteins including three structural proteins (capsid (C), premembrane (prM) and envelope (E)) which are incorporated into the viral particle, and seven non-structural proteins (NS1, NS2A, NS2B, NS3, NS4A, NS4B, NS5). The E protein interacts with cellular receptors and viral uptake occurs via receptor-mediated endocytosis followed by fusion of viral and endosomal membrane and release of the nucleocapsid into the cytoplasm. Translation and replication of the viral genome occurs in the cytoplasm in association with intracellular membranes. Particle assembly takes place in the endoplasmic reticulum and first leads to the formation of immature viruses with a rough surface formed by spikes of 60 trimers of prME heterodimers. In the acidic environment of the trans-Golgi network the trimeric spikes undergo a conformational change into 90 dimers and expose the prM protein cleavage site. The peptide pr is cleaved from prM by the cellular protease furin to form a smooth, mature virion virus particle with a herringbone-like arrangement of 90 E homodimers with T=3 pseudo-icosahedral symmetry. The prM cleavage allows E to adopt the conformational state required for its entry functions, i.e. receptor-binding and acidic-pH-induced membrane fusion after uptake by receptor-mediated endocytosis. NS1 is a multifunctional viral protein that is secreted as a hexamer by infected cells and circulates in the bloodstream. Inside infected cells, NS1 acts as a cofactor for viral replication and assembly, while the secreted form plays a role in immune evasion. NS1 has been shown to contribute to flavivirus pathogenisis by directly triggering endothelial hyperpermeability as well as by inducing the release of vasoactive cytokines from peripheral blood mono-nuclear cells (PBMCs) via activation of TLR, both leading to vascular leak: (Beatty et al., 2015, Modhiran et al., 2015). Dengue virus NS1 triggers endothelial permeability and vascular leak that is prevented by NS1 vaccination (Beatty et al 2015) NS1 is well conserved among flaviviruses, exhibiting 20%-40% identity and 60%-80% similarity (Song et al 2016). Therefore targeting YFV NS1 could potentially influence virus dissemination and pathogenesis by reducing vascular leak.

For example, YFV polyprotein of YFV strain 17D preferably comprises or consists of an amino acid sequence according to GenBank-ID NP_041726.1.

The term “YFV protein” as used herein typically refers to an individual structural or non-structural YFV protein. For example, YFV protein in the meaning of the present invention may be a protein selected from the group consisting of YFV capsid protein (C), YFV premembrane protein (prM), YFV premembrane envelope protein (prME) also referred herein as “Yellow fever virus prME polyprotein” or “YFV prME polyprotein”, YFV peptide pr (pr), YFV membrane protein (M), YFV envelope protein (E) and a YFV non-structural protein (NS).

As used herein, the term “YFV protein” may also refer to an amino acid sequence corresponding to an individual YFV protein as present in YFV polyprotein (precursor protein). Said amino acid sequence in the polyprotein may differ from the amino acid sequence of the respective mature YFV protein (i.e. after cleavage/processing the polyprotein). For example, the corresponding amino acid sequence comprised in the polyprotein may comprise amino acid residues that are removed during cleavage/processing of the polyprotein (such as a signal sequence/signal peptide or a target site(s) for a protease) and that are no longer present in the respective mature flavivirus protein. In the context of the present invention, the term “YFV protein” comprises both, the precursor amino acid sequence comprised in a YFV polyprotein (i.e. as part of a polypeptide chain optionally further comprising other viral proteins) as well as the respective mature individual YFV protein.

In the context of the present invention, the term “YFV protein” may also refer to YFV polyprotein or, more preferably, to a fragment of a YFV polyprotein, such as a YFV prME polyprotein, a YFV ME (e.g. a YFV ME) protein, or a NS1 (e.g. a YFV NS1). In this context, the term “YFV prME polyprotein” thus refers to a protein comprising an amino acid sequence corresponding to YFV prME polyprotein as comprised in a flavivirus polyprotein, or to a fragment or variant of a YFV prME polyprotein as comprised in a YFV polyprotein. Hence, the terms “YFV prME polyprotein or fragments or variants thereof” or “peptide or protein derived from a Yellow fever virus prME polyprotein” as used herein do not necessarily relate to full-length prME polyprotein, but preferably comprises at least a fragment of pr, M, E, ME, prM or prME. Also, the terms “YFV NS1 protein or fragments or variants thereof” or “peptide or protein derived from a Yellow fever virus NS1 protein” as used herein do not necessarily relate to full-length NS1 protein, but preferably comprises at least a fragment of NS1.

Where reference is made to amino acid (aa) residues and their position in a YFV protein or in a YFV polyprotein, any numbering used herein—unless stated otherwise—relates to the position of the respective aa residue in a corresponding YFV precursor polyprotein, wherein position “1” corresponds to the first aa residue, i.e. the aa residue at the N-terminus of a YFV precursor polyprotein. More preferably, the numbering with regard to aa residues refers to the respective position of an aa residue in a YFV precursor polyprotein, which is preferably derived from a YFV described herein.

In the following the amino acid (aa) regions of YFV proteins and fragments are indicated herein including the respective aa position in the YFV 17D precursor polyprotein (SEQ ID NO: 120). The following abbreviations are used herein with reference to YFV proteins throughout the specification (including information provided under the identifier <223> of the sequence listing): C: capsid protein C (e.g. aa 1-101); X: fragment of capsid protein C (N-terminal overhang, e.g. aa 92-101); SS: ER anchor/signal sequence/signal peptide (SS) for the capsid protein C (e.g. aa 102-121); pr: peptide pr (e.g. aa 122-210); M: matrix protein M (e.g. aa 211-285); prM: premembrane protein prM (e.g. aa 122-285); E: envelope protein E (e.g. aa 286-778); prME: premembrane envelope protein prME/prME polyprotein (e.g. aa 122-778); XX: fragment of non-structural protein NS1 (C-terminal overhang, e.g. aa 779-788); eSS: signal sequence for the envelope protein E (e.g. aa 758-778), NS1: non-structural protein 1 (e.g. aa 779-1130); Y: fragment of non-structural protein NS2A (c-terminal overhang, e.g. aa 1131-1177), NS2A: non-structural protein 2A (e.g. aa 1131-1354); NS2B: non-structural protein 2B (e.g. aa 1355-1484); NS3: non-structural protein 3 (e.g. aa 1485-2107); NS4A: non-structural protein 4A (e.g. aa 2108-2233); P2K: Peptide 2k (e.g. aa 2234-2256); NS4B: non-structural protein 4B (e.g. aa 2257-2506); NS5: non-structural protein 5 (e.g. aa 2507-3411).

Suitably, the at least one antigenic peptide or protein is derived from a YFV prME polyprotein, or a fragment or variant thereof.

In some embodiments, the at least one coding sequence encodes at least one antigenic peptide or protein derived from a Yellow fever virus non-structural protein, wherein said non-structural protein may be selected from NS1, NS2A, NS2B, NS3, NS4A, NS4B, NS5. In that context, NS1 is particularly preferred.

In preferred embodiments, the at least one coding sequence encodes at least one antigenic peptide or protein derived from a Yellow fever virus prME polyprotein is pr, M, E, ME, prM or prME, or a fragment or variant of any of these.

YFV prME is a polypeptide which comprises or consists of YFV premembrane (“prM”) and a YFV envelope (“E”) protein. The YFV (pre-) membrane protein ((pr)M) is a seven beta-stranded glycoprotein that facilitates E protein folding and regulates the oligomeric state of E proteins to prevent adventitious fusion during the egress of virus particles from infected cells. The expression of the E protein together with prM or M potentially allows for secretion of the E protein in the form of virus-like particles (VLP) and maintaining the integrity of neutralizing epitopes on E protein. The VLP are similar to infectious YFV virus particles in terms of structure but are safer as they are noninfectious. Accordingly, the artificial RNA comprises or consists of at least one coding sequence encoding at least one antigenic peptide or protein derived from a YFV prME polyprotein or a fragment or variant thereof.

In preferred embodiments, the at least one coding sequence encodes prME.

In that context, it has to be understood that advantageously “the at least one coding sequence encoding prME” also comprises a “signal sequence” or a secretory signal peptide (SS: ER anchor/signal sequence (SS) for the capsid protein C (e.g. aa 102-121). As used herein, the term “signal sequence” or “signal peptide” preferably refers to an amino acid sequence, which is involved in the targeting of a protein, e.g. a YFV protein, to a cellular compartment, preferably a membrane, more preferably a membrane of the endoplasmic reticulum (ER). A signal sequence in the context of the present invention preferably comprises from 3 to 40, 3 to 30, 3 to 20, 5 to 20 or 10 to 20 amino acid residues. Such a signal sequence may be present, for example, in a YFV polyprotein and may be removed during processing of said polyprotein in vivo. Suitably, YFV capsid protein (C) as present in a YFV polyprotein typically comprises a C-terminal signal sequence, corresponding to the amino acid sequence immediately N-terminal of YFV pr protein (e.g. amino acid residues aa 102-121 in a YFV polyprotein before cleavage).

Accordingly, in preferred embodiments, the at least one coding sequence encodes prME, in particular, SS-prME.

In other preferred embodiments, the at least one coding sequence encodes prME additionally comprising a C-terminal overhang comprising a fragment of YFV non-structural protein NS1 and/or an N-terminal overhang comprising a fragment of YFV capsid protein C.

The N-terminal overhang of the capsid protein C (“N-terminal overhang”, or “X”) may be beneficial for the correct translocation and orientation of the prM/E membrane protein into the membrane of the endoplasmic reticulum. N-terminal overhangs may comprise a fragment of YFV capsid protein C and may contain 5, 6, 7, 8, 9, 10, 11, 12, 13, 14, 15, 16, 17, 18, 19, 20, or more additional amino acid residues of the C-terminus of capsid protein C. Preferably, the N-terminal overhang comprises 9 additional amino acid residues of the C-terminus of capsid protein C and a methionine residue (e.g. 92-MRGLSSRKRR-101 of SEQ ID NO: 120). Accordingly, in preferred embodiments, the “N-terminal overhang” sequence contains five positively charged residues (K, R) which may be important for the anchoring of the prM/E protein. The C-terminal overhang of the amino terminus of NS1 (“C-terminal overhang” or “XX”) was included to facilitate the correct incorporation into the ER membrane and efficient processing of the polyprotein prM/E by the host signal peptidase. C-terminal overhangs may comprise a fragment of YFV non-structural protein NS1 may contain 5, 6, 7, 8, 9, 10, 11, 12, 13, 14, 15, 16, 17, 18, 19, 20, or more additional amino acid residues of the amino terminus of NS1. Preferably, the C-terminal overhang comprises 10 additional amino acid residues of the amino terminus of NS1 (e.g. 779-DQGCAINFGK-788 of SEQ ID NO: 120) that were suitably included to facilitate the correct incorporation into the ER membrane and efficient processing of the polyprotein prM/E by the host signal peptidase.

In embodiments, the at least one antigenic protein or peptide preferably comprises or consists of at least one of the following elements, or a fragment or variant thereof (explanation of abbreviations provided above): X; SS; pr; M; prM; E; prME; XX.

In preferred embodiments, the at least one coding sequence encodes prME, in particular, SS-prME or X-SS-prME-XX. In other embodiments, the at least one coding sequence encodes X-SS-prME or SS-prME-XX.

In embodiments, the artificial RNA as defined herein comprises at least one coding sequence encoding at least one antigenic peptide or protein derived from YFV comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 23-56, 541-586 of EP patent application EP17207141.7 or a fragment or variant of any of these sequences. In this context, SEQ ID NOs: 23-56, 541-586, the disclosure related thereto, and the description provided under the <223> in the sequence listing of EP patent application EP17207141.7 are herewith incorporated by reference.

In preferred embodiments, the artificial RNA of the first aspect comprises at least one coding sequence encoding at least one YFV prME comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 120-126 or a fragment or variant of any of these sequences. Additional information regarding each of these suitable amino acid sequences encoding YFV prME proteins may also be derived from the sequence listing, in particular from the details provided therein under identifier <223> as explained in the following.

For example, the numeric identifier <223> in the sequence listing of SEQ ID NO: 121 reads as follows: “derived and/or modified protein sequence (wt) from YFV_NC_002031.1_X-SS-prME-XX (polyprotein: aa92-788)”. It has to be noted that throughout the sequence listing, information provided under numeric identifier <223> follows the same structure: “<SEQUENCE_DESCRIPTOR> from <CONSTRUCT_IDENTIFIER>”.

The <SEQUENCE_DESCRIPTOR> relates to the type of sequence (e.g., “derived and/or modified protein sequence”, “derived and/or modified CDS” “mRNA product Design a-1 comprising derived and/or modified sequence”, or “mRNA product Design b-4 comprising derived and/or modified sequence”, or “mRNA product Design c-5 comprising derived and/or modified sequence”, or “mRNA product Design g-4 comprising derived and/or modified sequence” etc.) and whether the sequence comprises or consists of a wild type sequence (“wt”) or whether the sequence comprises or consists of a sequence-optimized sequence (e.g. “opt1”, “opt2”, “opt3”, “opt4”, “opt5”, “opt6”, “opt11”; sequence optimizations are described in further detail below).

The <CONSTRUCT_IDENTIFIER> provided under numeric identifier <223> has the following structures: (“organism_construct name”, or “organism_accession number_construct name”) and is intended to help the person skilled in the art to explicitly derive suitable nucleic acid sequences (e.g., RNA, mRNA) encoding the same YFV polyprotein according to the invention. For example, the <CONSTRUCT_IDENTIFIER> provided under numeric identifier <223> of SEQ ID NO: 121 reads as follows: “YFV_NC_002031.1 X-SS-prME-XX (polyprotein: aa92-788)”.

In that specific example, the respective protein sequence is derived from “Yellow Fever virus” (organism) with the NCBI accession number “NC_002031.1”, wherein the polyprotein comprises the structural elements “X-SS-prME-XX” (construct name). If the skilled person uses the construct identifier of SEQ ID NO: 121, namely “YFV_NC_002031.1_X-SS-prME-XX (polyprotein: aa92-788)”, said person easily arrives at the following list of SEQ ID NOs (amino acid sequences, nucleic acid coding sequences, mRNA sequences) derived from SEQ ID NO: 121 that can easily be retrieved from the sequence listing of the present invention:

### Amino Acid Sequence:

### SEQ ID NO: 121

### Nucleic Acid Coding Sequences:

### SEQ ID NOs: 158, 195, 232, 269, 306, 343, 380, 417, 454, 491

mRNA Sequences:
SEQ ID NOs: 528, 534, 540, 546, 552, 558, 564, 570, 576, 582, 588, 594, 600, 606, 612, 618, 624, 630, 636, 642, 648, 654, 660, 666, 672, 678, 684, 690, 696, 702, 708, 714, 720, 726, 732, 738, 744, 750, 756, 762, 768, 774, 780, 786, 792, 798, 804, 810, 816, 822, 828, 834, 840, 846, 852, 858, 864, 870, 876, 882, 888, 894, 900, 906, 912, 918, 924, 930, 936, 942, 948, 954, 960, 966, 972, 978, 984, 990, 996, 1002, 1008, 1014, 1020, 1026, 1032, 1038, 1044, 1050, 1056, 1062, 1068, 1074, 1080, 1086, 1092, 1098, 1104, 1110, 1116, 1122, 1128, 1134, 1140, 1146, 1152, 1158, 1164, 1170, 1176, 1182, 1188, 1194, 1200, 1206, 1212, 1218, 1224, 1230, 1236, 1242, 1248, 1254, 1260, 1266, 1272, 1278, 1284, 1290, 1296, 1302, 1308-1320, 1320, 2129, 2130, 2135, 2136, 2141, 2142, 2147, 2148, 2153, 2154, 2159, 2160, 2165, 2166, 2171, 2172, 2177, 2178, 2183, 2184, 2189, 2190, 2195, 2196

In preferred embodiments, the at least one coding sequence encodes the YFV non-structural protein NS1.

In that context, it has to be understood that advantageously “the at least one coding sequence encoding NS1” may also comprise a “signal sequence” or a secretory signal peptide (SS: ER anchor/signal sequence (eSS) for the envelope protein E (e.g. aa 758-778). As used herein, the term “signal sequence” or “signal peptide” preferably refers to an amino acid sequence, which is involved in the targeting of a protein, e.g. a YFV protein, to a cellular compartment, preferably a membrane, more preferably a membrane of the endoplasmic reticulum (ER). A signal sequence in the context of the present invention preferably comprises from 3 to 40, 3 to 30, 3 to 25, 5 to 25 or preferably 21 amino acid residues. Such a signal sequence may be present, for example, in a YFV polyprotein and may be removed during processing of said polyprotein in vivo. Suitably, YFV envelope protein (E) as present in a YFV polyprotein typically comprises a C-terminal signal sequence, corresponding to the amino acid sequence immediately N-terminal of YFV E protein (e.g. amino acid residues aa 758-778 in a YFV polyprotein before cleavage).

Accordingly, in preferred embodiments, the at least one coding sequence encodes NS1, in particular, eSS-NS1.

In other preferred embodiments, the at least one coding sequence encodes NS1 additionally comprising a C-terminal overhang comprising a fragment of YFV non-structural protein NS2A and/or an N-terminal overhang comprising a fragment of YFV envelope protein E.

The N-terminal overhang of the non-structural protein NS2A protein (“N-terminal overhang”, or “Y”) may be beneficial for the correct translocation and orientation of the NS1 protein into the membrane of the endoplasmic reticulum. The N-terminal overhang may thereby increase the surface expression of the encoded antigenic peptide or protein. N-terminal overhangs may comprise a fragment of NS2A and may contain 10-50, 20-50, 30-50, 40-50, 41, 42, 43, 44, 45, 46, 47, 48, 49, 50, or more additional amino acid residues of the C-terminus of the NS2A protein. Preferably, the N-terminal overhang comprises 47 additional amino acid residues of the C-terminus of the NS2A protein (e.g. 1131-GEIHAVPFGLVSMMIAMEVVLRKRQGPKQMLVGGVVLLGAMLVGQV-1177 of SEQ ID NO: 120)

In preferred embodiments, the at least one coding sequence encodes NS1, in particular, eSS-NS1, or eSS-NS1-Y.

In embodiments, the artificial RNA of the first aspect comprises at least one coding sequence encoding at least one YFV NS1 comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 2201, 2202, 2203, or 2204 or a fragment or variant of any of these sequences. Additional information regarding each of these suitable amino acid sequences encoding YFV NS1 proteins may also be derived from the sequence listing, in particular from the details provided therein under identifier <223> as explained above.

According to another preferred embodiment, the artificial RNA of the invention encodes at least one antigenic peptide or protein as defined above and may additionally encode at least one further heterologous peptide or protein element.

The following abbreviations for heterologous elements that may be part of YFV proteins of the invention are used throughout the specification (including information provided under the identifier <223> of the sequence listing): JEV: aa 400-500 (stem region) of the Japanese encephalitis virus envelope protein E; SSIgE: signal sequence of IgE; SSjev: signal sequence of Japanese encephalitis virus.

Suitably, the at least one further peptide or protein element may promote secretion of the encoded antigenic peptide or protein of the invention (e.g. via secretory signal sequences), promote anchoring of the encoded antigenic peptide or protein of the invention in the plasma membrane (e.g. via transmembrane elements), promote formation of antigen complexes (e.g. via multimerization domains), promote virus-like particle formation (VLP forming sequence). In addition, the artificial nucleic acid sequence according to the present invention may additionally encode peptide linker elements, self-cleaving peptides, immunologic adjuvant sequences or dendritic cell targeting sequences.

Suitable multimerization domains may be selected from the list of amino acid sequences according to SEQ ID NOs: 1116-1167 of the patent application WO2017/081082, or fragments or variants of these sequences. On nucleic acid level, any nucleic acid sequence (e.g. RNA sequence) may be selected which encodes such amino acid sequences. In this context, the disclosure of WO2017/081082 is herewith incorporated by reference. Suitable transmembrane elements may be selected from the list of amino acid sequences according to SEQ ID NOs: 1228-1343 of the patent application WO2017/081082, or fragments or variants of these sequences. On nucleic acid level, any nucleic acid sequence (e.g. RNA sequence) may be selected which encodes such amino acid sequences. In this context, the disclosure of WO2017/081082 is herewith incorporated by reference. Suitable VLP forming sequences may be selected from the list of amino acid sequences according to SEQ ID NOs: 1168-1227 of the patent application WO2017/081082, or fragments or variants of these sequences. On nucleic acid level, any nucleic acid sequence (e.g. RNA sequence) may be selected which encodes such amino acid sequences. In this context, the disclosure of WO2017/081082 is herewith incorporated by reference. Suitable peptide linkers may be selected from the list of amino acid sequences according to SEQ ID NOs: 1509-1565 of the patent application WO2017/081082, or fragments or variants of these sequences. On nucleic acid level, any nucleic acid sequence (e.g. RNA sequence) may be selected which encodes such amino acid sequences. In this context, the disclosure of WO2017/081082 is herewith incorporated by reference. Suitable self-cleaving peptides may be selected from the list of amino acid sequences according to SEQ ID NOs: 1434-1508 of the patent application WO2017/081082, or fragments or variants of these sequences. On nucleic acid level, any nucleic acid sequence (e.g. RNA sequence) may be selected which encodes such amino acid sequences. In this context, the disclosure of WO2017/081082 is herewith incorporated by reference. Suitable immunologic adjuvant sequences may be selected from the list of amino acid sequences according to SEQ ID NOs: 1360-1421 of the patent application WO2017/081082, or fragments or variants of these sequences. On nucleic acid level, any nucleic acid sequence (e.g. RNA sequence) may be selected which encodes such amino acid sequences. In this context, the disclosure of WO2017/081082 is herewith incorporated by reference. Suitable dendritic cell (DCs) targeting sequences may be selected from the list of amino acid sequences according to SEQ ID NOs: 1344-1359 of the patent application WO2017/081082, or fragments or variants of these sequences. On nucleic acid level, any nucleic acid sequence (e.g. RNA sequence) may be selected which encodes such amino acid sequences. In this context, the disclosure of WO2017/081082 is herewith incorporated by reference.

In a preferred embodiment, the RNA encodes at least one antigenic peptide or protein as defined herein and additionally a heterologous secretory signal sequence or heterologous secretory signal peptides. The heterologous secretory signal sequence may increase the secretion of the encoded antigenic peptide or protein.

Suitable secretory signal peptides may be selected from the list of amino acid sequences according to SEQ ID NOs: 1-1115 and SEQ ID NO: 1728 of the patent application WO2017/081082, or fragments or variants of these sequences. On nucleic acid level, any nucleic acid sequence (e.g. RNA sequence) may be selected which encodes such amino acid sequences. In this context, the disclosure of WO2017/081082 is herewith incorporated by reference.

In preferred embodiments, the artificial RNA encodes at least one antigenic peptide or protein as defined herein (e.g. prME or NS1) and additionally at least one heterologous secretory signal sequence, wherein the secretory signal sequence is preferably a signal sequence derived from IgE, or Japanese encephalitis virus (JEV).

In that context it has to be understood that in embodiments where heterologous signal sequences are used, the endogenous signal sequences has to be removed (e.g., “SS” of a “SS-prME-XX” construct has to be removed and exchanged by a heterologous signal sequence, e.g., IgE-leader, to generate an “SSIgE-prME-XX” construct; “SS” of a “SS-prME” construct has to be removed and exchanged by a heterologous signal sequence, e.g., IgE-leader, to generate an “SSIgE-prME construct”; “SS” of a “SS-prME-XX” construct has to be removed and exchanged by a heterologous signal sequence, e.g., SSjev, to generate an “SSjev-prME-XX” construct; “SS” of a “SS-prME” construct has to be removed and exchanged by a heterologous signal sequence, e.g., SSjev, to generate an “SSjev-prME” construct), e.g., SSjev(V3), to generate an “SSjev(V3)-prMEdelsstem_TM-JEV”construct; “X-SS” of a “X-SS-prMEdelstem_TM-JEV” construct has to be removed and exchanged by a heterologous signal sequence, e.g., SSjev(V3), to generate an “SSjev(V3)-prMEdelstem_TM-JEV” construct) or e.g. “eSS” of a “eSS-NS1” construct has to be removed and exchanged by a heterologous signal sequence, e.g., IgE-leader, to generate a “SSIgE-NS1” or “SSIgE-NS1-Y” construct).

According to preferred embodiments, the secretory signal sequence comprises an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 56-61, or 1330-1357 or a fragment or variant of any of these sequences. Additional information regarding each of these suitable amino acid sequences encoding secretory signal sequences may also be derived from the sequence listing, in particular from the details provided therein under identifier <223>.

Accordingly, in embodiments, the artificial RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 62-109, or 1358-1586 or a fragment or a fragment or variant of any of these sequences. Additional information regarding each of these suitable nucleic acid sequences encoding may also be derived from the sequence listing, in particular from the details provided therein under identifier <223>.

In preferred embodiments, the artificial RNA encodes at least one antigenic peptide or protein as defined herein and additionally at least one heterologous further virus element, preferably a JEV stem sequence.

In that context, the at least one antigenic peptide or protein encoded by the at least one coding region of the artificial RNA may encode YFV prME, or a fragment or variant thereof, wherein the stem region and/or the transmembrane domain may be deleted and/or replaced by a corresponding amino acid sequence derived from Japanese Encephalitis Virus (JEV: aa 400-500 (stem region) of the Japanese encephalitis virus envelope protein E) e.g., “XX” of a “SS-prME-XX” construct has to be removed and exchanged by a heterologous stem region sequence, e.g., JEV, to generate an “SS-prME-delstem_TM-JEV” construct.

The stem region connects domain III of the YFV envelope protein to the transmembrane domain. It is believed that the replacement of the endogenous stem region (and/or transmembrane domain) by a stem region (and/or transmembrane domain) derived from Japanese encephalitis virus (JEV) is capable of increasing the production of YFV virus-like particles.

In one embodiment, the at least one antigenic peptide or protein encoded by the at least one coding region of the artificial RNA comprises or consists of YFV prME, or a fragment or variant thereof, wherein the stem region and the transmembrane domain is replaced by the amino acid sequence derived from JEV (aa 400-500 (stem region) of the Japanese encephalitis virus envelope protein E).

According to preferred embodiments, the JEV stem sequence comprises an amino acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 110, or a fragment or variant of any of these sequences. Additional information regarding each of these suitable amino acid sequences encoding a JEV stem sequence may also be derived from the sequence listing, in particular from the details provided therein under identifier <223>.

Accordingly, in embodiments, the artificial RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 111-119 or a fragment or a fragment or variant of any of these sequences. Additional information regarding each of these suitable nucleic acid sequences encoding a JEV stem sequence may also be derived from the sequence listing, in particular from the details provided therein under identifier <223>.

In preferred embodiments, the artificial RNA of the first aspect comprises at least one coding sequence encoding at least one YFV prME and at least one heterologous element comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 127-157, 1587, 1588 or a fragment or variant of any of these sequences. Additional information regarding each of these suitable amino acid sequences encoding YFV prME proteins comprising heterologous signal sequences and/or JEV element may also be derived from the sequence listing, in particular from the details provided therein under identifier <223>.

In some embodiments, the artificial RNA of the first aspect comprises at least one coding sequence encoding at least one YFV NS1 and at least one heterologous element comprising or consisting of at least one amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NO: 2205 or a fragment or variant of thereof.

According to preferred embodiments, the artificial nucleic acid, particularly the artificial RNA comprises at least one coding sequence encoding at least one antigenic peptide or protein derived from YFV, preferably derived from YFV prME polyprotein or YFV NS1, or fragments and variants thereof. In that context, any coding sequence encoding at least one antigenic peptide or protein derived from YFV, preferably derived from YFV prME polyprotein or YFV NS1, or fragments and variants thereof may be understood as suitable coding sequence and may therefore be comprised in the artificial RNA of the invention.

In embodiments, the artificial RNA of the first aspect may comprise or consist of at least one coding sequence encoding at least one antigenic peptide or protein derived from YFV (e.g., C, M, prME, NS1, NS2A, NS3, NS5 etc.) preferably encoding any one of SEQ ID NOs: 23-56, 541-586 of EP patent application EP17207141.7 or a fragment or variant of any of these sequences. More preferably, the artificial RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 57-540, 587-954 of EP patent application EP17207141.7 or a fragment or variant of any of these sequences. In this context, SEQ ID NOs: 57-540, 587-954, the disclosure related thereto, and the description provided under the <223> in the sequence listing of patent application EP17207141.7 are herewith incorporated by reference.

In preferred embodiments, the artificial RNA of the first aspect may comprise or consist of at least one coding sequence encoding at least one antigenic peptide or protein derived from YFV prME as defined herein (e.g. SS-prME, X-SS-prME-XX, etc.), preferably encoding any one of SEQ ID NOs: 120-157, 1587, 1588 or fragments of variants thereof. It has to be understood that, on nucleic acid level, any nucleic acid sequence, in particular, any RNA sequence which encodes an amino acid sequences being identical to SEQ ID NOs: 120-157, 1587, 1588 or fragments or variants thereof, or any nucleic acid sequence (e.g. DNA sequence, RNA sequence) which encodes amino acid sequences being at least 70%, 80%, 85%, 86%, 87%, 88%, 89%, 90%, 91%, 92%, 93%, 94%, 95%, 96%, 97%, 98%, or 99% identical to any one of SEQ ID NOs: 120-157, 1587, 1588 or fragments or variants thereof, may be selected and may accordingly be understood as suitable coding sequence and may therefore be comprised in the artificial RNA of the first aspect of the invention.

In further embodiments, the artificial RNA of the first aspect may comprise or consist of at least one coding sequence encoding at least one antigenic peptide or protein derived from YFV NS1 as defined herein (e.g. eSS-NS1, SSIgE-NS1, eSS-NS1-Y, SSIgE-NS1-Y etc.), preferably encoding any one of SEQ ID NOs: 2201-2205 or fragments of variants thereof. It has to be understood that, on nucleic acid level, any nucleic acid sequence, in particular, any RNA sequence which encodes an amino acid sequences being identical to SEQ ID NOs: 2201-2205 or fragments or variants thereof, or any nucleic acid sequence (e.g. DNA sequence, RNA sequence) which encodes amino acid sequences being at least 70%, 80%, 85%, 86%, 87%, 88%, 89%, 90%, 91%, 92%, 93%, 94%, 95%, 96%, 97%, 98%, or 99% identical to any one of SEQ ID NOs: 2201-2205 or fragments or variants thereof, may be selected and may accordingly be understood as suitable coding sequence and may therefore be comprised in the artificial RNA of the first aspect of the invention.

Suitably, in particularly preferred embodiments, the artificial RNA of the first aspect comprises a coding sequence located between said 5′-UTR and said 3′-UTR, preferably downstream of said 5′-UTR and upstream of said 3′-UTR.

In preferred embodiments, the artificial RNA of the first aspect comprises a coding sequence that comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 158-1320, 1589-2200, 2206-2593 or a fragment or a fragment or variant of any of these sequences (see also Table 2AX, Table 2AY, Table 2BX and Table 2BY). Additional information regarding each of these suitable nucleic acid sequences encoding may also be derived from the sequence listing, in particular from the details provided therein under identifier <223>.

According to preferred embodiments, the artificial RNA is a modified and/or stabilized artificial RNA.

According to preferred embodiments, the artificial RNA of the present invention may thus be provided as a “stabilized artificial RNA” that is to say an RNA showing improved resistance to in vivo degradation and/or an artificial RNA showing improved stability in vivo, and/or an artificial RNA showing improved translatability in vivo. In the following, specific suitable modifications in this context are described which are suitably to “stabilize” the artificial RNA.

Such stabilization may be effected by providing a “dried RNA” and/or a “purified RNA” as specified herein. Alternatively or in addition to that, such stabilization can be effected, for example, by a modified phosphate backbone of the artificial RNA of the present invention. A backbone modification in connection with the present invention is a modification in which phosphates of the backbone of the nucleotides contained in the RNA are chemically modified. Nucleotides that may be preferably used in this connection contain e.g. a phosphorothioate-modified phosphate backbone, preferably at least one of the phosphate oxygens contained in the phosphate backbone being replaced by a sulfur atom. Stabilized RNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without implying any limitation, modifications from the group consisting of methylphosphonates, phosphoramidates and phosphorothioates (e.g. cytidine-5′-O-(1-thiophosphate)).

In the following, suitable modifications are described that are capable of “stabilizing” the artificial RNA of the invention.

According to embodiments, the artificial RNA according to the invention is a modified artificial RNA, wherein the modification refers to chemical modifications comprising backbone modifications as well as sugar modifications or base modifications.

In this context, a modified artificial RNA as defined herein may contain nucleotide analogues/modifications, e.g. backbone modifications, sugar modifications or base modifications. A backbone modification in connection with the present invention is a modification, in which phosphates of the backbone of the nucleotides contained in a nucleic acid, e.g. an artificial RNA, are chemically modified. A sugar modification in connection with the present invention is a chemical modification of the sugar of the nucleotides of the RNA as defined herein. Furthermore, a base modification in connection with the present invention is a chemical modification of the base moiety of the nucleotides of the RNA. In this context, nucleotide analogues or modifications are preferably selected from nucleotide analogues which are applicable for transcription and/or translation.

In particularly preferred embodiments of the present invention, the nucleotide analogues/modifications which may be incorporated into a modified nucleic acid or particularly into a modified RNA as described herein are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

Particularly preferred and suitable in the context of the invention are pseudouridine (ψ), N1-methylpseudouridine (m1ψ), 5-methylcytosine, and 5-methoxyuridine. Accordingly, the artificial RNA as defined herein may comprise at least one modified nucleotide selected from pseudouridine (ψ), N1-methylpseudouridine (m1ψ), 5-methylcytosine, and 5-methoxyuridine.

In preferred embodiments, the artificial RNA of the invention comprises at least one coding sequence, wherein the at least one coding sequence is a pseudouridine (ψ) modified coding sequence.

In further preferred embodiments, the artificial RNA of the invention comprises at least one coding sequence, wherein the at least one coding sequence is a N1-methylpseudouridine (m1ψ) modified coding sequence.

In preferred embodiments, the artificial RNA of the invention comprises at least one coding sequence, wherein the at least one coding sequence is a codon modified coding sequence.

In preferred embodiments, the at least one coding sequence of the invention is a codon modified coding sequence, wherein the amino acid sequence encoded by the at least one codon modified coding sequence is preferably not being modified compared to the amino acid sequence encoded by the corresponding wild type coding sequence.

The term “codon modified coding sequence” relates to coding sequences that differ in at least one codon (triplets of nucleotides coding for one amino acid) compared to the corresponding wild type coding sequence. Suitably, a codon modified coding sequence in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably (cf. Table 1) to optimize/modify the coding sequence for in vivo applications as outlined above.

In particularly preferred embodiments of the first aspect, the at least one sequence is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof, or any combination thereof.

According to preferred embodiments, the artificial RNA of the invention may be modified, wherein the C content of the at least one coding sequence may be increased, preferably maximized, compared to the C content of the corresponding wild type coding sequence (herein referred to as “C maximized coding sequence”). The amino acid sequence encoded by the C maximized coding sequence of the RNA is preferably not modified as compared to the amino acid sequence encoded by the respective wild type nucleic acid coding sequence. The generation of a C maximized nucleic acid sequences may suitably be carried out using a modification method according to WO2015/062738. In this context, the disclosure of WO2015/062738 is included herewith by reference. Throughout the disclosure of the invention, including the <223> identifier of the sequence listing, C maximized coding sequences of suitable flavivirus nucleic acid sequences are indicated by the abbreviation “opt2”.

According to embodiments, the artificial RNA of the present invention may be modified, wherein the G/C content of the at least one coding sequence of the invention may be modified compared to the G/C content of the corresponding wild type coding sequence (herein referred to as “G/C content modified coding sequence”). In this context, the terms “G/C optimization” or “G/C content modification” relate to a nucleic acid, preferably an artificial nucleic acid of the invention that comprises a modified, preferably an increased number of guanosine and/or cytosine nucleotides as compared to the corresponding wild type nucleic acid sequence. Such an increased number may be generated by substitution of codons containing adenosine or thymidine nucleotides by codons containing guanosine or cytosine nucleotides. If the enriched G/C content occurs in a coding sequence of DNA or RNA, it makes use of the degeneracy of the genetic code. In particular, in case of RNA, sequences having an increased G (guanosine)/C (cytosine) content are more stable than sequences having an increased A (adenosine)/U (uracil) content. The amino acid sequence encoded by the G/C content modified coding sequence of the nucleic acid sequence is preferably not modified as compared to the amino acid sequence encoded by the respective wild type nucleic acid coding sequence. Preferably, the G/C content of the coding sequence of the artificial nucleic acid sequence, e.g. the RNA sequence of the present invention is increased by at least 10%, preferably by at least 20%, more preferably by at least 30%, most preferably by at least 40% compared to the G/C content of the coding sequence of the corresponding wild type nucleic acid sequence (e.g. RNA sequence), which codes for a YFV antigen as defined herein or a fragment or variant thereof.

According to preferred embodiments, the artificial RNA of the present invention may be modified, wherein the G/C content of the at least one coding sequence of the invention may be optimized compared to the G/C content of the corresponding wild type coding sequence (herein referred to as “G/C content optimized coding sequence”). “Optimized” in that context refers to a coding sequence wherein the G/C content is preferably increased to the essentially highest possible G/C content. The amino acid sequence encoded by the G/C content optimized coding sequence of the nucleic acid sequence is preferably not modified as compared to the amino acid sequence encoded by the respective wild type nucleic acid coding sequence. The generation of a G/C content optimized nucleic RNA sequence may suitably be carried out using a G/C content optimization method according to WO2002/098443. In this context, the disclosure of WO2002/098443 is included in its full scope in the present invention. Throughout the disclosure of the invention, including the <223> identifier of the sequence listing, G/C optimized coding sequences of suitable flavivirus nucleic acid sequences are indicated by the abbreviation “opt1, opt5, opt6, opt11”.

According to embodiments, the artificial RNA of the invention may be modified, wherein the codons in the at least one coding sequence of the invention may be adapted to human codon usage (herein referred to as “human codon usage adapted coding sequence”). Codons encoding the same amino acid occur at different frequencies in a subject, e.g. a human. Accordingly, the coding sequence of the artificial RNA is preferably modified such that the frequency of the codons encoding the same amino acid corresponds to the naturally occurring frequency of that codon according to the human codon usage e.g. as shown in Table 1. For example, in the case of the amino acid Ala, the wild type coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see Table 1). Accordingly, such a procedure (as exemplified for Ala) is applied for each amino acid encoded by the coding sequence of the artificial nucleic acid of the invention to obtain sequences adapted to human codon usage. Throughout the disclosure of the invention, including the <223> identifier of the sequence listing, human codon usage adapted coding sequences of suitable flavivirus nucleic acid sequences are indicated by the abbreviation “opt3”.

TABLE 1
Human codon usaqe table with frequencies
indicated for each amino acid

| Amino acid | codon | frequency |
|---|---|---|
| Ala | GCG | 0.10 |
| Ala | GCA | 0.22 |
| Ala | GCT | 0.28 |
| Ala | GCC* | 0.40 |
| Cys | TGT | 0.42 |
| Cys | TGC* | 0.58 |
| Asp | GAT | 0.44 |
| Asp | GAC* | 0.56 |
| Glu | GAG* | 0.59 |
| Glu | GAA | 0.41 |
| Phe | TTT | 0.43 |
| Phe | TTC* | 0.57 |
| Gly | GGG | 0.23 |
| Gly | GGA | 0.26 |
| Gly | GGT | 0.18 |
| Gly | GGC* | 0.33 |
| His | CAT | 0.41 |
| His | CAC* | 0.59 |
| Ile | ATA | 0.14 |
| Ile | ATT | 0.35 |
| Ile | ATC* | 0.52 |
| Lys | AAG* | 0.60 |
| Lys | AAA | 0.40 |
| Leu | TTG | 0.12 |
| Leu | TTA | 0.06 |
| Leu | CTG* | 0.43 |
| Leu | CTA | 0.07 |
| Leu | CTT | 0.12 |
| Leu | CTC | 0.20 |
| Met | ATG* | 1 |
| Asn | AAT | 0.44 |
| Asn | AAC* | 0.56 |
| Pro | CCG | 0.11 |
| Pro | CCA | 0.27 |
| Pro | CCT | 0.29 |
| Pro | CCC* | 0.33 |
| Gln | CAG* | 0.73 |
| Gln | CAA | 0.27 |
| Arg | AGG | 0.22 |
| Arg | AGA* | 0.21 |
| Arg | CGG | 0.19 |
| Arg | CGA | 0.10 |
| Arg | CGT | 0.09 |
| Arg | CGC | 0.19 |
| Ser | AGT | 0.14 |
| Ser | AGC* | 0.25 |
| Ser | TCG | 0.06 |
| Ser | TCA | 0.15 |
| Ser | TCT | 0.18 |
| Ser | TCC | 0.23 |
| Thr | ACG | 0.12 |
| Thr | ACA | 0.27 |
| Thr | ACT | 0.23 |
| Thr | ACC* | 0.38 |
| Val | GTG* | 0.48 |
| Val | GTA | 0.10 |
| Val | GTT | 0.17 |
| Val | GTC | 0.25 |
| Trp | TGG* | 1 |
| Tyr | TAT | 0.42 |
| Tyr | TAC* | 0.58 |
| Stop | TGA* | 0.61 |
| Stop | TAG | 0.17 |
| Stop | TAA | 0.22 |

*most frequent human codon

According to embodiments, the artificial RNA of the present invention may be modified, wherein the codon adaptation index (CAI) may be increased or preferably maximised in the at least one coding sequence of the invention (herein referred to as “CAI maximized coding sequence”). Accordingly, it is preferred that all codons of the wild type nucleic acid sequence that are relatively rare in e.g. a human cell are exchanged for a respective codon that is frequent in the e.g. a human cell, wherein the frequent codon encodes the same amino acid as the relatively rare codon. Suitably, the most frequent codons are used for each encoded amino acid (see Table 1, most frequent human codons are marked with asterisks). Suitably, the artificial RNA of the invention comprises at least one coding sequence, wherein the codon adaptation index (CAI) of the at least one coding sequence is at least 0.5, at least 0.8, at least 0.9 or at least 0.95. Most preferably, the codon adaptation index (CAI) of the at least one coding sequence is 1. For example, in the case of the amino acid Ala, the wild type coding sequence is adapted in a way that the most frequent human codon “GCC” is always used for said amino acid. Accordingly, such a procedure (as exemplified for Ala) is applied for each amino acid encoded by the coding sequence of the artificial RNA of the invention to obtain CAI maximized coding sequences. Throughout the disclosure of the invention including the <223> identifier of the sequence listing, CAI maximized coding sequences of suitable flavivirus nucleic acid sequences are indicated by the abbreviation “opt4”.

Accordingly, in a particularly preferred embodiment, the artificial RNA of the first aspect comprises at least one coding sequence comprising a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 195-527, 1591-1608, or 2211-2245 or a fragment or variant of any of these sequences (see also Table 2AX and Table 2AY). Additional information regarding each of these suitable nucleic acid sequences encoding may also be derived from the sequence listing, in particular from the details provided therein under identifier <223>.

In particularly preferred embodiment, the artificial RNA of the first aspect comprises at least one coding sequence comprising a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to any one of the G/C optimized or G/C content modified nucleic acid sequence according to the SEQ ID NOs: 195-305, 417-527, 1591-1596, 1603-1608, 2211-2215, 2231-2245 or a fragment or variant of any of these sequences (see also Table 2AX and Table 2AY). Additional information regarding each of these suitable nucleic acid sequences encoding may also be derived from the sequence listing, in particular from the details provided therein under identifier <223>.

In preferred embodiment, the artificial RNA of the invention comprises at least one coding sequence comprising a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to any one of the human codon usage adapted nucleic acid sequence according to the SEQ ID NOs: 343-379, 1599, 1600, 2221-2225 or a fragment or variant of any of these sequences (see also Table 2AX and Table 2AY). Additional information regarding each of these suitable nucleic acid sequences encoding may also be derived from the sequence listing, in particular from the details provided therein under identifier <223>.

In particularly preferred embodiment, the artificial RNA of the first aspect comprises at least one coding sequence comprising a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to any one of the C maximized nucleic acid sequence according to the SEQ ID NOs: 306-342, 1597, 1598, 2216-2220 or a fragment or variant of any of these sequences (see also Table 2AX and Table 2AY). Additional information regarding each of these suitable nucleic acid sequences encoding may also be derived from the sequence listing, in particular from the details provided therein under identifier <223>.

In preferred embodiment, the artificial RNA of the invention comprises at least one coding sequence comprising a codon modified nucleic acid sequence which is identical or at least 70%, 80%, 85%, 86%, 87%, 88%, 89%, 90%, 91%, 92%, 93%, 94%, 95%, 96%, 97%, 98%, or 99% identical to any one of the CAI maximized nucleic acid sequence according to the SEQ ID NOs: 380-416, 1601, 1602, 2226-2230 or a fragment or variant of any of these sequences (see also Table 2AX and Table 2AY). Additional information regarding each of these suitable nucleic acid sequences encoding may also be derived from the sequence listing, in particular from the details provided therein under identifier <223>.

In embodiments, the A/U content in the environment of the ribosome binding site of the artificial nucleic acid, particularly the artificial RNA of the invention may be increased compared to the A/U content in the environment of the ribosome binding site of its respective wild type nucleic acid. This modification (an increased A/U content around the ribosome binding site) increases the efficiency of ribosome binding to the nucleic acid, preferably the RNA. An effective binding of the ribosomes to the ribosome binding site in turn has the effect of an efficient translation of the RNA. Accordingly, in a particularly preferred embodiment, the artificial nucleic acid of the invention comprises a ribosome binding site, also referred to as “Kozak sequence” identical to or at least 80%, 85%, 90%, 95% identical to any one of the sequences SEQ ID NOs: 29, 30 or fragments or variants thereof.

Preferred YFV polypeptide and nucleic acid coding sequences (CDS) are provided in Table 2AX. Therein, Columns A to I represent a specific suitable construct of the invention derived from prME of YFV 17D. The protein designs are indicated in row 1, the specific protein SEQ ID NOs as provided in the sequence listing are in row 2 (“protein”), e.g., for “SS-prME” (column A) the protein is SEQ ID NO: 125, 126. The SEQ ID NOs of corresponding wild type coding sequences for each protein construct are provided in row 3 (“CDS wt”), e.g., for “SS-prME” (column A) the protein is SEQ ID NO: 125, 126 and the wt coding sequence is SEQ ID NO: 162, 163. The SEQ ID NOs of corresponding codon modified coding sequences for each protein construct are provided in row 4 to row 10 (“CDS opt1”, “CDS opt2”, “CDS opt3”, “CDS opt4”, “CDS opt5”, “CDS opt6”, “CDS opt11”), e.g., for “SS-prME” (column A) the protein is SEQ ID NO: 125, 126 and the wt coding sequence is SEQ ID NO: 162, 163, and the codon modified coding sequences are SEQ ID NO: 199, 200, 236, 237, 273, 274 for CDS opt1, SEQ ID NO: 310, 311 for CDS opt2, SEQ ID NO: 347, 348 for CDS opt3, SEQ ID NO: 384, 385 for CDS opt4, SEQ ID NO: 421, 422 for CDS opt5, SEQ ID NO: 458, 459 for CDS opt6, and SEQ ID NO: 495, 496 for CDS opt11. Further information e.g. regarding the type of codon modified coding sequence is provided in the <223> identifier for each of the respective SEQ ID NO in the sequence listing.

TABLE 2AX
Preferred coding sequences encoding YFV prME

| | | | | | | | | G | H | |
|---|---|---|---|---|---|---|---|---|---|---|
| | | | | | | | | X-SS- | SSjev(V3)- | |
| | | | B | C | D | E | F | prMEdel- | prMEdel- | I |
| | | A | X-SS- | SSjev- | SSjev- | SSIgE- | SSIgE- | stem_TM- | stem_TM- | X-SS- |
| 1 | | SS-prME | prME-XX | prME | prME-XX | prME | prME-XX | JEV | JEV | prME |
| 2 | Protein | 125, 126 | 121 | 136, 137, | 134, 135, | 132, 133 | 130, 131 | 1587 | 1588 | 122 |
| | | | | 140, 141, | 138, 139, | | | | | |
| | | | | 144, 145, | 142, 143, | | | | | |
| | | | | 148, 149, | 146, 147, | | | | | |
| | | | | 152, 153 | 150, 151 | | | | | |
| 3 | CDS | 162, 163 | 158 | 173, 174, | 171, 172, | 169, 170 | 167, 168 | 1589 | 1590 | 159 |
| | wt | | | 177, 178, | 175, 176, | | | | | |
| | | | | 181, 182, | 179, 180, | | | | | |
| | | | | 185, 186, | 183, 184, | | | | | |
| | | | | 189, 190 | 187, 188 | | | | | |
| 4 | CDS | 199, 200, | 195, 232, | 210, 211, | 208, 209, | 206, 207, | 204, 205, | 1591, 1593, | 1592, 1594, | 196, 233, |
| | opt1 | 236, 237, | 269 | 214, 215, | 212, 213, | 243, 244, | 241, 242, | 1595 | | 270 |
| | | 273, 274 | | 218, 219, | 216, 217, | 280, 281 | 278, 279 | | | |
| | | | | 222, 223, | 220, 221, | | | | 1596 | |
| | | | | 226, 227, | 224, 225, | | | | | |
| | | | | 247, 248, | 245, 246, | | | | | |
| | | | | 251, 252, | 249, 250, | | | | | |
| | | | | 255, 256, | 253, 254, | | | | | |
| | | | | 259, 260, | 257, 258, | | | | | |
| | | | | 263, 264, | 261, 262, | | | | | |
| | | | | 284, 285, | 282, 283, | | | | | |
| | | | | 288, 289, | 286, 287, | | | | | |
| | | | | 292, 293, | 290, 291, | | | | | |
| | | | | 296, 297, | 294, 295, | | | | | |
| | | | | 300, 301 | 298, 299 | | | | | |
| 5 | CDS | 310, 311 | 306 | 321, 322, | 319, 320, | 317, 318 | 315, 316 | 1597 | 1598 | 307 |
| | opt2 | | | 325, 326, | 323, 324, | | | | | |
| | | | | 329, 330, | 327, 328, | | | | | |
| | | | | 333, 334, | 331, 332, | | | | | |
| | | | | 337, 338 | 335, 336 | | | | | |
| 6 | CDS | 347, 348 | 343 | 358, 359, | 356, 357, | 354, 355 | 352, 353 | 1599 | 1600 | 344 |
| | opt3 | | | 362, 363, | 360, 361, | | | | | |
| | | | | 366, 367, | 364, 365, | | | | | |
| | | | | 370, 371, | 368, 369, | | | | | |
| | | | | 374, 375 | 372, 373 | | | | | |
| 7 | CDS | 384, 385 | 380 | 395, 396, | 393, 394, | 391, 392 | 389, 390 | 1601 | 1602 | 381 |
| | opt4 | | | 399, 400, | 397, 398, | | | | | |
| | | | | 403, 404, | 401, 402, | | | | | |
| | | | | 407, 408, | 405, 406, | | | | | |
| | | | | 411, 412 | 409, 410 | | | | | |
| 8 | CDS | 421, 422 | 417 | 432, 433, | 430, 431, | 428, 429 | 426, 427 | 1603 | 1604 | 418 |
| | opt5 | | | 436, 437, | 434, 435, | | | | | |
| | | | | 440, 441, | 438, 439, | | | | | |
| | | | | 444, 445, | 442, 443, | | | | | |
| | | | | 448, 449 | 446, 447 | | | | | |
| 9 | CDS | 458, 459 | 454 | 469, 470, | 467, 468, | 465, 466 | 463, 464 | 1605 | 1606 | 455 |
| | opt6 | | | 473, 474, | 471, 472, | | | | | |
| | | | | 477, 478, | 475, 476, | | | | | |
| | | | | 481, 482, | 479, 480, | | | | | |
| | | | | 485, 486 | 483, 484 | | | | | |
| 10 | CDS | 495, 496 | 491 | 506, 507, | 504, 505, | 502, 503 | 500, 501 | 1607 | 1608 | 492 |
| | opt11 | | | 510, 511, | 508, 509, | | | | | |
| | | | | 514, 515, | 512, 513, | | | | | |
| | | | | 518, 519, | 516, 517, | | | | | |
| | | | | 522, 523 | 520, 521 | | | | | |

Further preferred YFV polypeptide and nucleic acid coding sequences (CDS) are provided in Table 2AY. Therein, Columns A to E represent a specific suitable construct of the invention derived from NS1 of YFV 17D. The protein designs are indicated in row 1, the specific protein SEQ ID NOs as provided in the sequence listing are in row 2 (“protein”), e.g., for “eSS-NS1-Y” (column A) the protein is SEQ ID NO: 2201. The SEQ ID NOs of corresponding wild type coding sequences for each protein construct are provided in row 3 (“CDS wt”), e.g., for “eSS-NS1-Y” (column A) the protein is SEQ ID NO: 2201 and the wt coding sequence is SEQ ID NO: 2206. The SEQ ID NOs of corresponding codon modified coding sequences for each protein construct are provided in row 4 to row 10 (“CDS opt1”, “CDS opt2”, “CDS opt3”, “CDS opt4”, “CDS opt5”, “CDS opt6”, “CDS opt11”), e.g., for “eSS-NS1-Y” (column A) the protein is SEQ ID NO: 2201 and the wt coding sequence is SEQ ID NO: 2206, and the codon modified coding sequences are SEQ ID NO: 2211 for CDS opt1, SEQ ID NO: 2216 for CDS opt2, SEQ ID NO: 2221 for CDS opt3, SEQ ID NO: 2226 for CDS opt4, SEQ ID NO: 2231 for CDS opt5, SEQ ID NO: 2236 for CDS opt6, and SEQ ID NO: 2241 for CDS opt11. Further information e.g. regarding the type of codon modified coding sequence is provided in the <223> identifier for each of the respective SEQ ID NO in the sequence listing.

TABLE 2AY
Preferred coding sequences encoding YFV NS1

| | | A | B | | | E |
|---|---|---|---|---|---|---|
| | | eSS- | eSS- | C | D | SSIgE- |
| 1 | | NS1-Y | NS1 | NS1-Y | NS1 | NS1 |
| 2 | Protein | 2201 | 2202 | 2203 | 2204 | 2205 |
| 3 | CDS wt | 2206 | 2207 | 2208 | 2209 | 2210 |
| 4 | CDS opt1 | 2211 | 2212 | 2213 | 2214 | 2215 |
| 5 | CDS opt2 | 2216 | 2217 | 2218 | 2219 | 2220 |
| 6 | CDS opt3 | 2221 | 2222 | 2223 | 2224 | 2225 |
| 7 | CDS opt4 | 2226 | 2227 | 2228 | 2229 | 2230 |
| 8 | CDS opt5 | 2231 | 2232 | 2233 | 2234 | 2235 |
| 9 | CDS opt6 | 2236 | 2237 | 2238 | 2239 | 2240 |
| 10 | CDS opt11 | 2241 | 2242 | 2243 | 2244 | 2245 |

In embodiments, the artificial RNA of the first aspect is monocistronic, bicistronic, or multicistronic.

In preferred embodiments, the artificial RNA of the invention is monocistronic.

The term “monocistronic nucleic acid” or “monocistronic nucleic acid” will be recognized and understood by the person of ordinary skill in the art, and is for example intended to refer to an artificial RNA that comprises only one coding sequences as defined herein. The terms “bicistronic nucleic acid, multicistronic nucleic acid” or “monocistronic RNA” as used herein will be recognized and understood by the person of ordinary skill in the art, and are for example intended to refer to an artificial RNA that may have two (bicistronic) or even more (multicistronic) coding sequences.

In embodiments, the artificial RNA of the invention is monocistronic and the coding sequence of said monocistronic artificial RNA encodes at least two different antigenic peptides or proteins derived from YFV prME as defined herein, or a fragment or variant thereof. Accordingly, the at least one coding sequence of the monocistronic artificial RNA may encode at least two, three, four, five, six, seven, eight and more antigenic peptides or proteins derived from a YFV, preferably a YFV prME as defined herein linked with or without an amino acid linker sequence, wherein said linker sequence can comprise rigid linkers, flexible linkers, cleavable linkers (e.g. self-cleaving peptides) as defined above, or a combination thereof (herein referred to as “multi-antigen-constructs/nucleic acid”).

In embodiments, the artificial RNA of the invention is bicistronic or multicistronic and comprises at least two coding sequences, wherein the at least two coding sequences encode two or more different antigenic peptides or proteins derived from YFV, preferably YFV prME and/or NS1 as defined herein, or a fragment or variant of any of these. Accordingly, the coding sequences in a bicistronic or multicistronic artificial RNA suitably encodes distinct antigenic proteins or peptides as defined herein or a fragment or variant thereof. Preferably, the coding sequences in said bicistronic or multicistronic artificial RNA may be separated by at least one IRES (internal ribosomal entry site) sequence. Thus, the term “encoding two or more antigenic peptides or proteins” may mean, without being limited thereto, that the bicistronic or multicistronic artificial RNA encodes e.g. at least two, three, four, five, six or more (preferably different) antigenic peptides or proteins of different YFV or their fragments or variants within the definitions provided herein. Alternatively, the bicistronic or multicistronic artificial RNA may encode e.g. at least two, three, four, five, six or more (preferably different) antigenic peptides or proteins derived from the same YFV or fragments or variants within the definitions provided herein. In that context, suitable IRES sequences may be selected from the list of nucleic acid sequences according to SEQ ID NOs: 1566-1662 of the patent application WO2017/081082, or fragments or variants of these sequences. In this context, the disclosure of WO2017/081082 relating to IRES sequences is herewith incorporated by reference.

It has to be understood that in the context of the invention, certain combinations of coding sequences may be generated by any combination of monocistronic, bicistronic and multicistronic artificial nucleic acids and/or multi-antigen-constructs/nucleic acid to obtain a nucleic acid composition encoding multiple antigenic peptides or proteins as defined herein.

Preferably, the artificial RNA comprising at least one coding sequence as defined herein typically comprises a length of about 50 to about 20000, or 500 to about 20000 nucleotides, or about 500 to about 20000 nucleotides, or about 500 to about 10000 nucleotides, or of about 1000 to about 10000 nucleotides, or preferably of about 1000 to about 5000 nucleotides, or even more preferably of about 1000 to about 2500 nucleotides. According to preferred embodiments, the artificial RNA of the first aspect may be an mRNA, a self-replicating RNA, a circular RNA, or a replicon RNA.

In embodiments, the artificial RNA is a circular RNA. As used herein, “circular RNA” has to be understood as a circular polynucleotide that can encode at least one antigenic peptide or protein as defined herein. Accordingly, in preferred embodiments, said circular RNA comprises at least one coding sequence encoding at least one antigenic peptide or protein derived from YFV or a fragment or variant thereof as defined herein. The production of circRNAs can be performed using various methods provided in the art. For example, U.S. Pat. No. 6,210,931 teaches a method of synthesizing circRNAs by inserting DNA fragments into a plasmid containing sequences having the capability of spontaneous cleavage and self-circularization. U.S. Pat. No. 5,773,244 teaches producing circRNAs by making a DNA construct encoding an RNA cyclase ribozyme, expressing the DNA construct as an RNA, and then allowing the RNA to self-splice, which produces a circRNA free from intron in vitro. WO1992001813 teaches a process of making single strand circular nucleic acids by synthesizing a linear polynucleotide, combining the linear nucleotide with a complementary linking oligonucleotide under hybridization conditions, and ligating the linear polynucleotide. The person skilled in the art may also use methods provided in WO2015/034925 or WO2016/011222 to produce circular RNA. Accordingly, methods for producing circular RNA as provided in U.S. Pat. Nos. 6,210,931, 5,773,244, WO1992/001813, WO2015/034925 and WO2016/011222 are incorporated herewith by reference.

In embodiments, the artificial RNA is a replicon RNA. The term “replicon RNA” will be recognized and understood by the person of ordinary skill in the art, and are for example intended to be optimized self-replicating artificial RNA constructs. Such constructs include replication elements (replicase) derived from alphaviruses and the substitution of the structural virus proteins with the artificial nucleic acid of interest (in the context of the invention, an artificial nucleic acid comprising at least one coding sequence encoding at least one antigenic peptide or protein derived from YFV. Alternatively, the replicase may be provided on an independent construct comprising a replicase RNA sequence derived from e.g. Semliki forest virus (SFV), Sindbis virus (SIN), Venezuelan equine Encephalitis virus (VEE), Ross-River virus (RRV), or other viruses belonging to the alphavirus family. Downstream of the replicase lies a sub-genomic promoter that controls replication of the artificial nucleic acid of the invention, i.e. an artificial nucleic acid comprising at least one coding sequence encoding at least one antigenic peptide or protein derived from YFV.

In preferred embodiments, the artificial RNA of the first aspect is an mRNA.

The terms “RNA” and “mRNA” will be recognized and understood by the person of ordinary skill in the art, and are for example intended to be a ribonucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. The mRNA (messenger RNA) usually provides the nucleotide sequence that may be translated into an amino-acid sequence of a particular peptide or protein.

The artificial RNA, preferably the mRNA of the invention may be prepared using any method known in the art, including chemical synthesis such as e.g. solid phase RNA synthesis, as well as in vitro methods, such as RNA in vitro transcription reactions.

In a preferred embodiment, the artificial RNA, preferably the mRNA is obtained by RNA in vitro transcription.

Accordingly, the RNA of the invention is preferably an in vitro transcribed RNA.

The terms “RNA in vitro transcription” or “in vitro transcription” relate to a process wherein RNA is synthesized in a cell-free system (in vitro). RNA may be obtained by DNA-dependent in vitro transcription of an appropriate DNA template, which according to the present invention is a linearized plasmid DNA template or a PCR-amplified DNA template. The promoter for controlling RNA in vitro transcription can be any promoter for any DNA-dependent RNA polymerase. Particular examples of DNA-dependent RNA polymerases are the T7, T3, SP6, or Syn5 RNA polymerases. In a preferred embodiment of the present invention the DNA template is linearized with a suitable restriction enzyme, before it is subjected to RNA in vitro transcription.

Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein (e.g. m7G(5′)ppp(5′)G (m7G), m7G(5′)ppp(5′)(2′OMeG)pG or m7G(5′)ppp(5′)(2′OMeA)pG); optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCl2, which supplies Mg2+ ions as a co-factor for the polymerase; a buffer (TRIS or HEPES) to maintain a suitable pH value, which can also contain antioxidants (e.g. DTT), and/or polyamines such as spermidine at optimal concentrations, e.g. a buffer system comprising TRIS-Citrate as disclosed in WO2017/109161.

In embodiments, the nucleotide mixture used in RNA in vitro transcription may additionally contain modified nucleotides as defined herein. In that context, preferred modified nucleotides comprise pseudouridine (ip), N1-methylpseudouridine (m1ψ), 5-methylcytosine, and/or 5-methoxyuridine.

In preferred embodiments, the nucleotide mixture (i.e. the fraction of each nucleotide in the mixture) used for RNA in vitro transcription reactions may be optimized for the given RNA sequence, preferably as described WO2015/188933.

In embodiment where more than one different artificial RNA as defined herein has to be produced, e.g. where 2, 3, 4, 5, 6, 7, 8, 9, 10 or even more different artificial RNAs have to be produced (e.g. encoding different YFV prME antigens or NS1 antigens), procedures as described in WO2017/109134 may be suitably used.

In the context of RNA vaccine production, it may be required to provide GMP-grade RNA. GMP-grade RNA may be produced using a manufacturing process approved by regulatory authorities. Accordingly, in a particularly preferred embodiment, RNA production is performed under current good manufacturing practice (GMP), implementing various quality control steps on DNA and RNA level, preferably according to WO2016/180430. In preferred embodiments, the RNA of the invention is a GMP-grade RNA, particularly a GMP-grade mRNA.

In embodiments where multivalent mRNA compositions are used, said multivalent mRNA compositions are preferably produced according to procedures as disclosed in WO2017/1090134A1. In short, YFV DNA constructs (each of which comprising different YFV coding sequences and a T7 promotor; e.g. a DNA sequence coding for YFV prME and a DNA sequence coding for YFV NS1) may be used as a matrix for simultaneous PCR amplification. The obtained PCR product mixture may subsequently be purified and used as a template for simultaneous RNA in vitro transcription to generate a mixture of YFV mRNA constructs. The obtained YFV mRNA mixture may be subjected to quantitative and qualitative measurements (e.g., RNA AGE, RT-qPCR, NGS, and Spectrometry). Following that, purification may suitably be performed and formulation may be performed (e.g., protamine formulation, preferably LNP formulation).

The obtained RNA products are preferably purified using PureMessenger® (CureVac, Tubingen, Germany; RP-HPLC according to WO2008/077592) and/or tangential flow filtration (as described in WO2016/193206).

In a further preferred embodiment, the RNA, particularly the purified RNA, is lyophilized according to WO2016/165831 or WO2011/069586 to yield a temperature stable dried artificial RNA (powder) as defined herein. The RNA of the invention, particularly the purified RNA may also be dried using spray-drying or spray-freeze drying according to WO2016/184575 or WO2016/184576 to yield a temperature stable RNA (powder) as defined herein. Accordingly, in the context of manufacturing and purifying RNA, the disclosures of WO2017/109161, WO2015/188933, WO2016/180430, WO2008/077592, WO2016/193206, WO2016/165831, WO2011/069586, WO2016/184575, and WO2016/184576 are incorporated herewith by reference.

Accordingly, in preferred embodiments, the RNA is a dried RNA, particularly a dried mRNA.

The term “dried RNA” as used herein has to be understood as RNA that has been lyophilized, or spray-dried, or spray-freeze dried as defined above to obtain a temperature stable dried RNA (powder).

In preferred embodiments, the artificial RNA of the invention is a purified RNA, particularly purified mRNA.

The term “purified RNA” or “purified mRNA” as used herein has to be understood as RNA which has a higher purity after certain purification steps (e.g. HPLC, TFF, Oligo d(T) purification, precipitation steps) than the starting material (e.g. in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA fragments, abortive sequences etc.), free nucleotides (modified nucleotides, conventional NTPs, cap analogue), template DNA fragments, buffer components (HEPES, TRIS, MgCl2) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. It is also desirable for the degree of RNA purity that the amount of full length RNA transcripts is as close as possible to 100%. Accordingly “purified RNA” as used herein has a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target RNA and the total area of all peaks representing the by-products. Alternatively, the degree of purity may for example be determined by an analytical agarose gel electrophoresis or capillary gel electrophoresis.

It has to be understood that “dried RNA” as defined herein and “purified RNA” as defined herein or “GMP-grade mRNA” as defined herein may have superior stability characteristics (in vitro, in vivo) and improved efficiency (e.g. better translatability of the mRNA in vivo) and are therefore particularly suitable in the context of the invention.

The artificial RNA may suitably be modified by the addition of a 5′-cap structure, which preferably stabilizes the nucleic acid as described herein.

Accordingly, in preferred embodiments, the artificial RNA of the first aspect comprises a 5′-cap structure, preferably m7G, cap0 (e.g. m7G(5′)ppp(5′)G), cap1 (e.g. m7G(5′)ppp(5′)(2′OMeG) or m7G(5′)ppp(5′)(2′OMeA)), cap2, a modified cap0, or a modified cap1 structure.

The term “5′-cap structure” as used herein will be recognized and understood by the person of ordinary skill in the art, and is for example intended to refer to a modified nucleotide (cap analogue), particularly a guanine nucleotide, added to the 5′-end of an RNA molecule, e.g. an mRNA molecule. Preferably, the 5′-cap is added using a 5′-5′-triphosphate linkage (also named m7GpppN). Further examples of 5′-cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

Further 5′-cap structures which may be suitable in the context of the present invention are cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may also be formed in chemical RNA synthesis or, preferably, RNA in vitro transcription (co-transcriptional capping) using cap analogues.

The term “cap analogue” as used herein will be recognized and understood by the person of ordinary skill in the art, and is for example intended to refer to a non-polymerizable di-nucleotide that has cap functionality in that it facilitates translation or localization, and/or prevents degradation of a nucleic acid molecule, particularly of an RNA molecule, when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008/016473, WO2008/157688, WO2009/149253, WO2011/015347, and WO2013/059475). Further suitable cap analogons in that context are described in WO2017/066793, WO2017/066781, WO2017/066791, WO2017/066789, WO2017/066782, and WO2017/066797 wherein the disclosures referring to cap analogues are incorporated herewith by reference.

In embodiments, a modified cap1 structure is generated using tri-nucleotide cap analogue as disclosed in WO2017/053297, WO2017/066793, WO2017/066781, WO2017/066791, WO2017/066789, WO2017/066782, WO2018075827 and WO2017/066797. In particular, any cap structures derivable from the structure disclosed in claim 1-5 of WO2017/053297 may be suitably used to co-transcriptionally generate a modified cap1 structure. Further, any cap structures derivable from the structure defined in claim 1 or claim 21 of WO2018075827 may be suitably used to co-transcriptionally generate a modified cap1 structure.

The 5′-cap structure may suitably be added co-transcriptionally using cap-analogues as defined herein in an RNA in vitro transcription reaction as defined herein.

Preferred cap-analogues are the di-nucleotide cap analogues m7G(5′)ppp(5′)G (m7G) or 3′-O-Me-m7G(5′)ppp(5′)G to co-transcriptionally generate cap0 structures. Particularly preferred cap-analogues are the tri-nucleotide cap analogues m7G(5′)ppp(5′)(2′OMeA)pG or m7G(5′)ppp(5′)(2′OMeG)pG to co-transcriptionally generate cap1 structures,

In that context, it is preferred that the RNA of the invention comprises a Cap1 structure as defined above, which preferably results in an increased protein expression through e.g. high capping efficiencies and increased translation efficiencies. Further suitably, the RNA of the invention comprising a Cap1 structure displays a decreased stimulation of the innate immune system as compared to Cap0 constructs of the same nucleic acid sequence. The person of ordinary skill knows how to determine translation efficiencies, capping degree, and immune stimulation.

In other embodiments, the 5′-cap structure is added via enzymatic capping using capping enzymes (e.g. vaccinia virus capping enzymes, commercially available capping kits) to generate cap0 or cap1 or cap2 structures. In other embodiments, the 5′-cap structure (cap0, cap1) is added via enzymatic capping using immobilized capping enzymes, e.g. using a capping reactor (WO2016/193226).

In a particularly preferred embodiment, the artificial RNA of the first aspect of the invention comprises a cap1 structure, wherein said cap1 structure may be formed enzymatically or co-transcriptionally (e.g. using m7G(5′)ppp(5)(2′OMeA)pG or m7G(5′)ppp(5′)(2′OMeG)pG analogues).

In preferred embodiments, the artificial RNA of the first aspect comprises an m7G(5′)ppp(5′)(2′OMeA)pG cap structure. In such embodiments, the coding RNA comprises a 5′ terminal m7G cap, and an additional methylation of the ribose of the adjacent nucleotide of m7GpppN, in that case, a 2′O methylated adenosine.

In other preferred embodiments, the artificial RNA of the first aspect comprises an m7G(5′)ppp(5′)(2′OMeG)pG cap structure. In such embodiments, the coding RNA comprises a 5′ terminal m7G cap, and an additional methylation of the ribose of the adjacent nucleotide, in that case, a 2′O methylated guanosine.

Accordingly, whenever reference is made to suitable RNA or mRNA sequences in the context of the invention, the first nucleotide of said RNA or mRNA sequence, that is the nucleotide downstream of the m7G(5′)ppp structure, may be a 2′O methylated guanosine or a 2′O methylated adenosine.

Accordingly, in other embodiments, the artificial RNA of the invention may comprise a 5′-cap sequence element according to SEQ ID NOs: 31 or 1321, or a fragment or variant thereof.

In preferred embodiments, the artificial RNA of the invention comprises at least one poly(A) sequence, preferably comprising 30 to 150 adenosine nucleotides.

In preferred embodiments, the poly(A) sequence, suitable located at the 3′ terminus, comprises 10 to 500 adenosine nucleotides, 10 to 200 adenosine nucleotides, 40 to 200 adenosine nucleotides or 40 to 150 adenosine nucleotides. In a particularly preferred embodiment, the poly(A) sequence comprises about 64 adenosine nucleotides. In further particularly preferred embodiments, the poly(A) sequence comprises about 75 adenosine nucleotides. In further particularly preferred embodiments, the poly(A) sequence comprises about 100 adenosine nucleotides.

The terms “poly(A) sequence”, “poly(A) tail” or “3′-poly(A) tail” as used herein will be recognized and understood by the person of ordinary skill in the art, and are for example intended to be a sequence of adenosine nucleotides, typically located at the 3′-end of an RNA, of up to about 1000 adenosine nucleotides. In the context of the present invention, a poly(A) sequence may be located within an mRNA or any other nucleic acid molecule, such as, e.g., in a DNA vector, for example, in a vector serving as template for the generation of an RNA, preferably an mRNA, e.g., by transcription said vector.

Preferably, the poly(A) sequence of the artificial RNA is obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using commercially available polyadenylation kits and corresponding protocols known in the art, or alternatively, by using immobilized poly(A)polymerases e.g. using a polyadenylation reactor (as described in WO2016/174271).

In embodiments, the artificial RNA of the invention may contain a poly(A) sequence derived from a vector and may comprise at least one additional poly(A) sequence generated by enzymatic polyadenylation, e.g. as described in WO2016/091391.

In preferred embodiments, the artificial RNA of the invention comprises at least one poly(C) sequence, preferably comprising 10 to 40 cytosine nucleotides.

In preferred embodiments, the poly(C) sequence, suitable located at the 3′ terminus, comprises 10 to 200 cytosine nucleotides, 10 to 100 cytosine nucleotides, 20 to 70 cytosine nucleotides, 20 to 60 cytosine nucleotides, or 10 to 40 cytosine nucleotides. In a particularly preferred embodiment, the poly(C) sequence comprises about 30 cytosine nucleotides.

The term “poly(C) sequence” as used herein will be recognized and understood by the person of ordinary skill in the art, and are for example intended to be a sequence of cytosine nucleotides, typically located at the 3′-end of an RNA, of up to about 200 cytosine nucleotides. In the context of the present invention, a poly(C) sequence may be located within an mRNA or any other nucleic acid molecule, such as, e.g., in a DNA vector, for example, in a vector serving as template for the generation of an RNA, preferably an mRNA, e.g., by transcription of the vector.

Preferably, the poly(C) sequence in the RNA sequence of the present invention is derived from a DNA template by RNA in vitro transcription. In other embodiments, the poly(C) sequence is obtained in vitro by common methods of chemical synthesis without being necessarily transcribed from a DNA template.

In preferred embodiments, the artificial RNA of the first aspect comprises at least one histone stem-loop.

The term “histone stem-loop” as used herein will be recognized and understood by the person of ordinary skill in the art, and are for example intended to refer to nucleic acid sequences that are predominantly found in histone mRNAs. Exemplary histone stem-loop sequences are described in Lopez et al. (Davila Lopez et al, (2008), RNA, 14(1)). The stem-loops in histone pre-mRNAs are typically followed by a purine-rich sequence known as the histone downstream element (HDE). These pre-mRNAs are processed in the nucleus by a single endonucleolytic cleavage approximately 5 nucleotides downstream of the stem-loop, catalysed by the U7 snRNP through base pairing of the U7 snRNA with the HDE.

Histone stem-loop sequences/structures may suitably be selected from histone stem-loop sequences as disclosed in WO2012/019780, the disclosure relating to histone stem-loop sequences/structures incorporated herewith by reference. A histone stem-loop sequence that may be used within the present invention may preferably be derived from Formulae (I) or (II) of the patent application WO2012/019780. According to a further preferred embodiment the RNA as defined herein may comprise at least one histone stem-loop sequence derived from at least one of the specific Formulae (Ia) or (IIa) of the patent application WO2012/019780.

In particularly preferred embodiment, the artificial RNA of the invention comprises at least one histone stem-loop, wherein said histone stem-loop comprises a nucleic acid sequence according to SEQ ID NOs: 27, 28 or a fragments or variant thereof.

In further embodiments, the artificial RNA of the invention comprises a 3′-terminal sequence element. Said 3′-terminal sequence element has to be understood as a sequence element comprising a poly(A)sequence and a histone-stem-loop sequence, wherein said sequence element is located at the 3′ terminus of the artificial RNA of the invention.

In other embodiments, the artificial RNA of the invention may comprise a 3′-terminal sequence element according to SEQ ID NOs: 32-51, 1323-1329 or a fragment or variant thereof.

In preferred embodiments of the first aspect, the artificial RNA, preferably mRNA comprises preferably in 5′- to 3′-direction the following elements a)-h):
a) 5′-cap structure, preferably as specified herein;
b) optionally, 5′-UTR, preferably as specified herein;
c) at least one coding sequence, preferably as specified herein;
d) 3′-UTR, preferably as specified herein;
e) optionally, a poly(A) sequence, preferably as specified herein;
f) optionally, a poly(C) sequence, preferably as specified herein;
g) optionally, a histone stem-loop, preferably as specified herein;
h) optionally, a 3′-terminal sequence element as specified herein.

In further preferred embodiments of the first aspect, the artificial RNA, preferably mRNA comprises the following elements
a) 5′-cap structure, preferably as specified herein;
b) a 5′-UTR and a 3′-UTR element according to a-1, a-2, a-4, b-4, c-5, d-1, d-5, g-4, h-4, h-5, i-2, or i-3 as specified herein;
c) at least one coding sequence as specified herein, wherein said coding sequence is located between said 5′-UTR and said 3′-UTR, preferably downstream of said 5′-UTR and upstream of said 3′-UTR;
d) optionally, a poly(A) sequence, preferably as specified herein;
e) optionally, poly(C) sequence, preferably as specified herein;
f) optionally, histone stem-loop, preferably as specified herein;
g) optionally, a 3′-terminal sequence element as specified herein.

In further preferred embodiments of the first aspect, the artificial RNA, preferably mRNA comprises the following elements:
a) 5′-cap structure, preferably as specified herein;
b) a 5′-UTR and a 3′-UTR element according to a-1, a-4, b-4, d-5, c-5, i-3, or g-4 as specified herein;
c) at least one coding sequence as specified herein, wherein said coding region is located between said 5′-UTR and said 3′-UTR, preferably downstream of said 5′-UTR and upstream of said 3′-UTR;
d) optionally, a poly(A) sequence, preferably as specified herein;
e) optionally, poly(C) sequence, preferably as specified herein;
f) optionally, histone stem-loop, preferably as specified herein;
g) optionally, a 3′-terminal sequence element as specified herein.

In preferred embodiments of the first aspect, the artificial RNA, preferably mRNA comprises preferably in 5′- to 3′-direction the following elements a)-g):
a) 5′-cap structure, preferably as specified herein;
b) optionally, 5′-UTR, preferably as specified herein;
c) at least one coding sequence, preferably as specified herein;
d) 3′-UTR, preferably as specified herein;
e) optionally, a histone stem-loop, preferably as specified herein;
f) optionally, a poly(A) sequence, preferably as specified herein;
g) optionally, a 3′-terminal sequence element as specified herein.

In further preferred embodiments of the first aspect, the artificial RNA, preferably mRNA comprises the following elements
a) 5′-cap structure, preferably as specified herein;
b) a 5′-UTR and a 3′-UTR element according to a-1, a-2, a-4, b-4, c-5, d-1, d-5, g-4, h-4, h-5, i-2, or i-3 as specified herein;
c) at least one coding sequence as specified herein, wherein said coding sequence is located between said 5′-UTR and said 3′-UTR, preferably downstream of said 5′-UTR and upstream of said 3′-UTR;
d) optionally, histone stem-loop, preferably as specified herein;
e) optionally, a poly(A) sequence, preferably as specified herein;
f) optionally, a 3′-terminal sequence element as specified herein.

In further preferred embodiments of the first aspect, the artificial RNA, preferably mRNA comprises the following elements:
a) 5′-cap structure, preferably as specified herein;
b) a 5′-UTR and a 3′-UTR element according to a-1, a-4, b-4, d-5, c-5, i-3, or g-4 as specified herein;
c) at least one coding sequence as specified herein, wherein said coding region is located between said 5′-UTR and said 3′-UTR, preferably downstream of said 5′-UTR and upstream of said 3′-UTR;
d) optionally, histone stem-loop, preferably as specified herein;
e) optionally, a poly(A) sequence, preferably as specified herein;
f) optionally, a 3′-terminal sequence element as specified herein.

Preferred YFV polypeptide, nucleic acid and mRNA sequences are provided in Table 2BX. Therein, Columns A to I represent a specific suitable construct of the invention derived from prME of YFV 17D. The protein designs are indicated in row 1, the specific protein SEQ ID NOs as provided in the sequence listing are in row 2 (“protein”), e.g., for “SS-prME” (column A) the protein is SEQ ID NOs: 125, 126. The SEQ ID NOs of corresponding wild type coding sequences for each protein construct are provided in row 3 (“CDS wt”), e.g., for “SS-prME” (column A) the protein is SEQ ID NOs: 125, 126 and the wt coding sequence is SEQ ID NOs: 162, 163. The SEQ ID NOs of corresponding codon modified coding sequences (opt1, opt2, opt3, opt4, opt5, opt6, opt11 etc) for each protein construct are provided in row 4 (“CDS opt”), e.g., for “SS-prME” (column A) the protein is SEQ ID NOs: 125, 126 and the wt coding sequence is SEQ ID NOs: 162, 163, and the codon modified coding sequences are SEQ ID NOs: 199, 200, 236, 237, 273, 274, 310, 311, 347, 348, 384, 385, 421, 422, 458, 459, 495, 496. Further information e.g. regarding the type of codon modified coding sequence (opt1, opt2, opt3, opt4, opt5, opt6, opt11 etc.) is provided in the <223> identifier of the respective SEQ ID NO in the sequence listing. The SEQ ID NOs of corresponding mRNA constructs comprising said coding sequences comprising suitable 5′-UTRs and 3′-UTRs according to the invention are provided in rows 5 to 16 (row 5: “mRNA UTR a-1”; row 6: “mRNA UTR a-2”; row 7: “mRNA UTR b-4”; row 8: “mRNA UTR c-5”; row 9: “mRNA UTR d-1”; row 10: “mRNA UTR d-5”; row 11: “mRNA UTR g-4”; row 12: “mRNA UTR h-4”; row 13: “mRNA UTR h-5”; row 14: “mRNA UTR i-2”; row 15: “mRNA UTR i-3” row 16 “mRNA UTR a-4”). For example, for “SS-prME” (column A) the protein has an amino acid sequence according to SEQ ID NOs: 125, 126 and the wt coding sequence has a nucleic acid sequence according to SEQ ID NOs: 162, 163, and the codon modified coding sequences has a nucleic acid sequence according to SEQ ID NOs: 199, 200, 236, 237, 273, 274, 310, 311, 347, 348, 384, 385, 421, 422, 458, 459, 495, 496, and the mRNA sequences with e.g. UTR a-1 combination has an RNA sequence according to SEQ ID NOs: 529, 535, 541, 547, 553, 559, 565, 571, 577, 583. Further information e.g. regarding the type of coding sequence (wt, opt1, opt2, opt3, opt4, opt5, opt6, opt11 etc.) comprised in the mRNA constructs is provided in the <223> identifier of the respective SEQ ID NO in the sequence listing.

TABLE 2BX
mRNA constructs encoding YFV prME

| | | | | | | | | G | H | |
|---|---|---|---|---|---|---|---|---|---|---|
| | | | | | | | | X-SS- | SSjev(V3)- | |
| | | | B | C | D | E | F | prMEdel- | prMEdel- | I |
| | | A | X-SS- | SSjev- | SSjev- | SSIgE- | SSIgE- | stem_TM- | stem_TM- | X-SS- |
| 1 | | SS-prME | prME-XX | prME | prME-XX | prME | prME-XX | JEV | JEV | prME |
| 2 | Protein | 125, 126 | 121 | 136, 137, | 134, 135, | 132, 133 | 130, 131 | 1587 | 1588 | 122 |
| | | | | 140, 141, | 138, 139, | | | | | |
| | | | | 144, 145, | 142, 143, | | | | | |
| | | | | 148, 149, | 146, 147, | | | | | |
| | | | | 152, 153 | 150, 151 | | | | | |
| 3 | CDS | 162, 163 | 158 | 173, 174, | 171, 172, | 169, 170 | 167, 168 | 1589 | 1590 | 159 |
| | wt | | | 177, 178, | 175, 176, | | | | | |
| | | | | 181, 182, | 179, 180, | | | | | |
| | | | | 185, 186, | 183, 184, | | | | | |
| | | | | 189, 190 | 187, 188 | | | | | |
| 4 | CDS | 199, 200, | 195, 232, | 210, 211, | 208, 209, | 206, 207, | 204, 205, | 1591, 1593, | 1592, 1594, | 196, 233, |
| | opt | 236, 237, | 269, 306, | 214, 215, | 212, 213, | 243, 244, | 241, 242, | 1595, 1597, | 1596, 1598, | 270 |
| | | 273, 274, | 343, 380, | 218, 219, | 216, 217, | 280, 281, | 278, 279, | 1599, 1601, | 1600, 1602, | |
| | | 310, 311, | 417, 454, | 222, 223, | 220, 221, | 317, 318, | 315, 316, | 1603, 1605, | 1604, 1606, | |
| | | 347, 348, | 491 | 226, 227, | 224, 225, | 354, 355, | 352, 353, | 1607 | 1608 | |
| | | 384, 385, | | 247, 248, | 245, 246, | 391, 392, | 389, 390, | | | |
| | | 421, 422, | | 251, 252, | 249, 250, | 428, 429, | 426, 427, | | | |
| | | 458, 459, | | 255, 256, | 253, 254, | 465, 466, | 463, 464, | | | |
| | | 495, 496 | | 259, 260, | 257, 258, | 502, 503 | 500, 501 | | | |
| | | | | 263, 264, | 261, 262, | | | | | |
| | | | | 284, 285, | 282, 283, | | | | | |
| | | | | 288, 289, | 286, 287, | | | | | |
| | | | | 292, 293, | 290, 291, | | | | | |
| | | | | 296, 297, | 294, 295, | | | | | |
| | | | | 300, 301, | 298, 299, | | | | | |
| | | | | 321, 322, | 319, 320, | | | | | |
| | | | | 325, 326, | 323, 324, | | | | | |
| | | | | 329, 330, | 327, 328, | | | | | |
| | | | | 333, 334, | 331, 332, | | | | | |
| | | | | 337, 338, | 335, 336, | | | | | |
| | | | | 358, 359, | 356, 357, | | | | | |
| | | | | 362, 363, | 360, 361, | | | | | |
| | | | | 366, 367, | 364, 365, | | | | | |
| | | | | 370, 371, | 368, 369, | | | | | |
| | | | | 374, 375, | 372, 373, | | | | | |
| | | | | 395, 396, | 393, 394, | | | | | |
| | | | | 399, 400, | 397, 398, | | | | | |
| | | | | 403, 404, | 401, 402, | | | | | |
| | | | | 407, 408, | 405, 406, | | | | | |
| | | | | 411, 412, | 409, 410, | | | | | |
| | | | | 432, 433, | 430, 431, | | | | | |
| | | | | 436, 437, | 434, 435, | | | | | |
| | | | | 440, 441, | 438, 439, | | | | | |
| | | | | 444, 445, | 442, 443, | | | | | |
| | | | | 448, 449, | 446, 447, | | | | | |
| | | | | 469, 470, | 467, 468, | | | | | |
| | | | | 473, 474, | 471, 472, | | | | | |
| | | | | 477, 478, | 475, 476, | | | | | |
| | | | | 481, 482, | 479, 480, | | | | | |
| | | | | 485, 486, | 483, 484, | | | | | |
| | | | | 506, 507, | 504, 505, | | | | | |
| | | | | 510, 511, | 508, 509, | | | | | |
| | | | | 514, 515, | 512, 513, | | | | | |
| | | | | 518, 519, | 516, 517, | | | | | |
| | | | | 522, 523 | 520, 521 | | | | | |
| 5 | mRNA | 529, 535, | 528, 534, | 533, 539, | 532, 538, | 531, 537, | 530, 536, | 1609, 1613, | 1610, 1614, | 1611, 1615, |
| | UTR | 541, 547, | 540, 546, | 545, 551, | 544, 550, | 543, 549, | 542, 548, | 1617, 1621, | 1618, 1622, | 1619, 1623, |
| | a-1 | 553, 559, | 552, 558, | 557, 563, | 556, 562, | 555, 561, | 554, 560, | 1625, 1629, | 1626, 1630, | 1627, 1631, |
| | | 565, 571, | 564, 570, | 569, 575, | 568, 574, | 567, 573, | 566, 572, | 1633, 1637, | 1634, 1638, | 1635, 1639, |
| | | 577, 583 | 576, 582, | 581, 587 | 580, 586, | 579, 585 | 578, 584 | 1641, 1645, | 1642, 1646, | 1643, 1647, |
| | | | 1308, 2129, | | 1612, 1616, | | | 2133, 2169, | 2134, 2170, | 2131, 2167, |
| | | | 2130, 2141, | | 1620, 1624, | | | 2145, 2181, | 2146, 2182, | 2143, 2179, |
| | | | 2142, 2153, | | 1628, 1632, | | | 2157, 2193 | 2158, 2194 | 2155, 2191 |
| | | | 2154, 2165, | | 1636, 1640, | | | | | |
| | | | 2166, 2177, | | 1644, 1648, | | | | | |
| | | | 2178, 2189, | | 2132, 2168, | | | | | |
| | | | 2190 | | 2144, 2180, | | | | | |
| | | | | | 2156, 2192 | | | | | |
| 6 | mRNA | 589, 595, | 588, 594, | 593, 599, | 592, 598, | 591, 597, | 590, 596, | 1649, 1653, | 1650, 1654, | 1651, 1655, |
| | UTR | 601, 607, | 600, 606, | 605, 611, | 604, 610, | 603, 609, | 602, 608, | 1657, 1661, | 1658, 1662, | 1659, 1663, |
| | a-2 | 613, 619, | 612, 618, | 617, 623, | 616, 622, | 615, 621, | 614, 620, | 1665, 1669, | 1666, 1670, | 1667, 1671, |
| | | 625, 631, | 624, 630, | 629, 635, | 628, 634, | 627, 633, | 626, 632, | 1673, 1677, | 1674, 1678, | 1675, 1679, |
| | | 637, 643 | 636, 642, | 641, 647 | 640, 646, | 639, 645 | 638, 644 | 1681, 1685 | 1682, 1686 | 1683, 1687 |
| | | | 1309 | | 1652, 1656, | | | | | |
| | | | | | 1660, 1664, | | | | | |
| | | | | | 1668, 1672, | | | | | |
| | | | | | 1676, 1680, | | | | | |
| | | | | | 1684, 1688 | | | | | |
| 7 | mRNA | 649, 655, | 648, 654, | 653, 659, | 652, 658, | 651, 657, | 650, 656, | 1689, 1693, | 1690, 1694, | 1691, 1695, |
| | UTR | 661, 667, | 660, 666, | 665, 671, | 664, 670, | 663, 669, | 662, 668, | 1697, 1701, | 1698, 1702, | 1699, 1703, |
| | b-4 | 673, 679, | 672, 678, | 677, 683, | 676, 682, | 675, 681, | 674, 680, | 1705, 1709, | 1706, 1710, | 1707, 1711, |
| | | 685, 691, | 684, 690, | 689, 695, | 688, 694, | 687, 693, | 686, 692, | 1713, 1717, | 1714, 1718, | 1715, 1719, |
| | | 697, 703 | 696, 702, | 701, 707 | 700, 706, | 699, 705 | 698, 704 | 1721, 1725 | 1722, 1726 | 1723, 1727 |
| | | | 1310 | | 1692, 1696, | | | | | |
| | | | | | 1700, 1704, | | | | | |
| | | | | | 1708, 1712, | | | | | |
| | | | | | 1716, 1720, | | | | | |
| | | | | | 1724, 1728 | | | | | |
| 8 | mRNA | 709, 715, | 708, 714, | 713, 719, | 712, 718, | 711, 717, | 710, 716, | 1729, 1733, | 1730, 1734, | 1731, 1735, |
| | UTR | 721, 727, | 720, 726, | 725, 731, | 724, 730, | 723, 729, | 722, 728, | 1737, 1741, | 1738, 1742, | 1739, 1743, |
| | c-5 | 733, 739, | 732, 738, | 737, 743, | 736, 742, | 735, 741, | 734, 740, | 1745, 1749, | 1746, 1750, | 1747, 1751, |
| | | 745, 751, | 744, 750, | 749, 755, | 748, 754, | 747, 753, | 746, 752, | 1753, 1757, | 1754, 1758, | 1755, 1759, |
| | | 757, 763 | 756, 762, | 761, 767 | 760, 766, | 759, 765 | 758, 764 | 1761, 1765 | 1762, 1766 | 1763, 1767 |
| | | | 1311 | | 1732, 1736, | | | | | |
| | | | | | 1740, 1744, | | | | | |
| | | | | | 1748, 1752, | | | | | |
| | | | | | 1756, 1760, | | | | | |
| | | | | | 1764, 1768 | | | | | |
| 9 | mRNA | 769, 775, | 768, 774, | 773, 779, | 772, 778, | 771, 777, | 770, 776, | 1769, 1773, | 1770, 1774, | 1771, 1775, |
| | UTR | 781, 787, | 780, 786, | 785, 791, | 784, 790, | 783, 789, | 782, 788, | 1777, 1781, | 1778, 1782, | 1779, 1783, |
| | d-1 | 793, 799, | 792, 798, | 797, 803, | 796, 802, | 795, 801, | 794, 800, | 1785, 1789, | 1786, 1790, | 1787, 1791, |
| | | 805, 811, | 804, 810, | 809, 815, | 808, 814, | 807, 813, | 806, 812, | 1793, 1797, | 1794, 1798, | 1795, 1799, |
| | | 817, 823 | 816, 822, | 821, 827 | 820, 826, | 819, 825 | 818, 824 | 1801, 1805 | 1802, 1806 | 1803, 1807 |
| | | | 1312 | | 1772, 1776, | | | | | |
| | | | | | 1780, 1784, | | | | | |
| | | | | | 1788, 1792, | | | | | |
| | | | | | 1796, 1800, | | | | | |
| | | | | | 1804, 1808 | | | | | |
| 10 | mRNA | 829, 835, | 828, 834, | 833, 839, | 832, 838, | 831, 837, | 830, 836, | 1809, 1813, | 1810, 1814, | 1811, 1815, |
| | UTR | 841, 847, | 840, 846, | 845, 851, | 844, 850, | 843, 849, | 842, 848, | 1817, 1821, | 1818, 1822, | 1819, 1823, |
| | d-5 | 853, 859, | 852, 858, | 857, 863, | 856, 862, | 855, 861, | 854, 860, | 1825, 1829, | 1826, 1830, | 1827, 1831, |
| | | 865, 871, | 864, 870, | 869, 875, | 868, 874, | 867, 873, | 866, 872, | 1833, 1837, | 1834, 1838, | 1835, 1839, |
| | | 877, 883 | 876, 882, | 881, 887 | 880, 886, | 879, 885 | 878, 884 | 1841, 1845 | 1842, 1846 | 1843, 1847 |
| | | | 1313 | | 1812, 1816, | | | | | |
| | | | | | 1820, 1824, | | | | | |
| | | | | | 1828, 1832, | | | | | |
| | | | | | 1836, 1840, | | | | | |
| | | | | | 1844, 1848 | | | | | |
| 11 | mRNA | 889, 895, | 888, 894, | 893, 899, | 892, 898, | 891, 897, | 890, 896, | 1849, 1853, | 1850, 1854, | 1851, 1855, |
| | UTR | 901, 907, | 900, 906, | 905, 911, | 904, 910, | 903, 909, | 902, 908, | 1857, 1861, | 1858, 1862, | 1859, 1863, |
| | g-4 | 913, 919, | 912, 918, | 917, 923, | 916, 922, | 915, 921, | 914, 920, | 1865, 1869, | 1866, 1870, | 1867, 1871, |
| | | 925, 931, | 924, 930, | 929, 935, | 928, 934, | 927, 933, | 926, 932, | 1873, 1877, | 1874, 1878, | 1875, 1879, |
| | | 937, 943 | 936, 942, | 941, 947 | 940, 946, | 939, 945 | 938, 944 | 1881, 1885 | 1882, 1886 | 1883, 1887 |
| | | | 1314 | | 1852, 1856, | | | | | |
| | | | | | 1860, 1864, | | | | | |
| | | | | | 1868, 1872, | | | | | |
| | | | | | 1876, 1880, | | | | | |
| | | | | | 1884, 1888 | | | | | |
| 12 | mRNA | 949, 955, | 948, 954, | 953, 959, | 952, 958, | 951, 957, | 950, 956, | 1889, 1893, | 1890, 1894, | 1891, 1895, |
| | UTR | 961, 967, | 960, 966, | 965, 971, | 964, 970, | 963, 969, | 962, 968, | 1897, 1901, | 1898, 1902, | 1899, 1903, |
| | h-4 | 973, 979, | 972, 978, | 977, 983, | 976, 982, | 975, 981, | 974, 980, | 1905, 1909, | 1906, 1910, | 1907, 1911, |
| | | 985, 991, | 984, 990, | 989, 995, | 988, 994, | 987, 993, | 986, 992, | 1913, 1917, | 1914, 1918, | 1915, 1919, |
| | | 997, 1003 | 996, 1002, | 1001, 1007 | 1000, 1006, | 999, 1005 | 998, 1004 | 1921, 1925 | 1922, 1926 | 1923, 1927 |
| | | | 1315 | | 1892, 1896, | | | | | |
| | | | | | 1900, 1904, | | | | | |
| | | | | | 1908, 1912, | | | | | |
| | | | | | 1916, 1920, | | | | | |
| | | | | | 1924, 1928 | | | | | |
| 13 | mRNA | 1009, 1015, | 1008, 1014, | 1013, 1019, | 1012, 1018, | 1011, 1017, | 1010, 1016, | 1929, 1933, | 1930, 1934, | 1931, 1935, |
| | UTR | 1021, 1027, | 1020, 1026, | 1025, 1031, | 1024, 1030, | 1023, 1029, | 1022, 1028, | 1937, 1941, | 1938, 1942, | 1939, 1943, |
| | h-5 | 1033, 1039, | 1032, 1038, | 1037, 1043, | 1036, 1042, | 1035, 1041, | 1034, 1040, | 1945, 1949, | 1946, 1950, | 1947, 1951, |
| | | 1045, 1051, | 1044, 1050, | 1049, 1055, | 1048, 1054, | 1047, 1053, | 1046, 1052, | 1953, 1957, | 1954, 1958, | 1955, 1959, |
| | | 1057, 1063 | 1056, 1062, | 1061, 1067 | 1060, 1066, | 1059, 1065 | 1058, 1064 | 1961, 1965 | 1962, 1966 | 1963, 1967 |
| | | | 1316 | | 1932, 1936, | | | | | |
| | | | | | 1940, 1944, | | | | | |
| | | | | | 1948, 1952, | | | | | |
| | | | | | 1956, 1960, | | | | | |
| | | | | | 1964, 1968 | | | | | |
| 14 | mRNA | 1069, 1075, | 1068, 1074, | 1073, 1079, | 1072, 1078, | 1071, 1077, | 1070, 1076, | 1969, 1973, | 1970, 1974, | 1971, 1975, |
| | UTR | 1081, 1087, | 1080, 1086, | 1085, 1091, | 1084, 1090, | 1083, 1089, | 1082, 1088, | 1977, 1981, | 1978, 1982, | 1979, 1983, |
| | i-2 | 1093, 1099, | 1092, 1098, | 1097, 1103, | 1096, 1102, | 1095, 1101, | 1094, 1100, | 1985, 1989, | 1986, 1990, | 1987, 1991, |
| | | 1105, 1111, | 1104, 1110, | 1109, 1115, | 1108, 1114, | 1107, 1113, | 1106, 1112, | 1993, 1997, | 1994, 1998, | 1995, 1999, |
| | | 1117, 1123, | 1116, 1122, | 1121, 1127, | 1120, 1126, | 1119, 1125, | 1118, 1124, | 2001, 2005, | 2002, 2006, | 2003, 2007, |
| | | 1129, 1135, | 1128, 1134, | 1133, 1139, | 1132, 1138, | 1131, 1137, | 1130, 1136, | 2009, 2013, | 2010, 2014, | 2011, 2015, |
| | | 1141, 1147, | 1140, 1146, | 1145, 1151, | 1144, 1150, | 1143, 1149, | 1142, 1148, | 2017, 2021, | 2018, 2022, | 2019, 2023, |
| | | 1153, 1159, | 1152, 1158, | 1157, 1163, | 1156, 1162, | 1155, 1161, | 1154, 1160, | 2025, 2029, | 2026, 2030, | 2027, 2031, |
| | | 1165, 1171, | 1164, 1170, | 1169, 1175, | 1168, 1174, | 1167, 1173, | 1166, 1172, | 2033, 2037, | 2034, 2038, | 2035, 2039, |
| | | 1177, 1183 | 1176, 1182, | 1181, 1187 | 1180, 1186, | 1179, 1185 | 1178, 1184 | 2041, 2045 | 2042, 2046 | 2043, 2047 |
| | | | 1317, 1318 | | 1972, 1976, | | | | | |
| | | | | | 1980, 1984, | | | | | |
| | | | | | 1988, 1992, | | | | | |
| | | | | | 1996, 2000, | | | | | |
| | | | | | 2004, 2008, | | | | | |
| | | | | | 2012, 2016, | | | | | |
| | | | | | 2020, 2024, | | | | | |
| | | | | | 2028, 2032, | | | | | |
| | | | | | 2036, 2040, | | | | | |
| | | | | | 2044, 2048 | | | | | |
| 15 | mRNA | 1189, 1195, | 1188, 1194, | 1193, 1199, | 1192, 1198, | 1191, 1197, | 1190, 1196, | 2049, 2053, | 2050, 2054, | 2051, 2055, |
| | UTR | 1201, 1207, | 1200, 1206, | 1205, 1211, | 1204, 1210, | 1203, 1209, | 1202, 1208, | 2057, 2061, | 2058, 2062, | 2059, 2063, |
| | i-3 | 1213, 1219, | 1212, 1218, | 1217, 1223, | 1216, 1222, | 1215, 1221, | 1214, 1220, | 2065, 2069, | 2066, 2070, | 2067, 2071, |
| | | 1225, 1231, | 1224, 1230, | 1229, 1235, | 1228, 1234, | 1227, 1233, | 1226, 1232, | 2073, 2077, | 2074, 2078, | 2075, 2079, |
| | | 1237, 1243 | 1236, 1242, | 1241, 1247 | 1240, 1246, | 1239, 1245 | 1238, 1244 | 2081, 2085, | 2082, 2086, | 2083, 2087, |
| | | | 1319, 2135, | | 2052, 2056, | | | 2139, 2175, | 2140, 2176, | 2137, 2173, |
| | | | 2136, 2147, | | 2060, 2064, | | | 2151, 2187, | 2152, 2188, | 2149, 2185, |
| | | | 2148, 2159, | | 2068, 2072, | | | 2163, 2199 | 2164, 2200 | 2161, 2197 |
| | | | 2160, 2171, | | 2076, 2080, | | | | | |
| | | | 2172, 2183, | | 2084, 2088, | | | | | |
| | | | 2184, 2195, | | 2138, 2174, | | | | | |
| | | | 2196 | | 2150, 2186, | | | | | |
| | | | | | 2162, 2198 | | | | | |
| 16 | mRNA | 1249, 1255, | 1248, 1254, | 1253, 1259, | 1252, 1258, | 1251, 1257, | 1250, 1256, | 2089, 2093, | 2090, 2094, | 2091, 2095, |
| | UTR | 1261, 1267, | 1260, 1266, | 1265, 1271, | 1264, 1270, | 1263, 1269, | 1262, 1268, | 2097, 2101, | 2098, 2102, | 2099, 2103, |
| | a-4 | 1273, 1279, | 1272, 1278, | 1277, 1283, | 1276, 1282, | 1275, 1281, | 1274, 1280, | 2105, 2109, | 2106, 2110, | 2107, 2111, |
| | | 1285, 1291, | 1284, 1290, | 1289, 1295, | 1288, 1294, | 1287, 1293, | 1286, 1292, | 2113, 2117, | 2114, 2118, | 2115, 2119, |
| | | 1297, 1303 | 1296, 1302, | 1301, 1307 | 1300, 1306, | 1299, 1305 | 1298, 1304 | 2121, 2125 | 2122, 2126 | 2123, 2127 |
| | | | 1320 | | 2092, 2096, | | | | | |
| | | | | | 2100, 2104, | | | | | |
| | | | | | 2108, 2112, | | | | | |
| | | | | | 2116, 2120, | | | | | |
| | | | | | 2124, 2128 | | | | | |

In preferred embodiments, the artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 528-1307, 1609-2200 or a fragment or variant of any of these sequences.

In particularly preferred embodiments, the artificial RNA comprises

(a) at least one heterologous 5′ untranslated region (5′-UTR) and/or at least one heterologous 3′ untranslated region (3′-UTR) and
(b) at least one coding sequence operably linked to said 3′-UTR and/or 5′-UTR encoding at least one antigenic peptide or protein derived from a YFV prME polyprotein or a fragment or variant thereof, wherein
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 528-587, 1609-1648, 2129-2134, 2141-2146, 2153-2158, 2165-2170, 2177-2182, 2189-2194 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 1248-1307, 2089-2128 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 648-707, 1689-1728 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 708-767, 1729-1768 or a fragment or variant of any of these sequences; or
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 888-947, 1849-1888 or a fragment or variant of any of these sequences.

Further preferred YFV polypeptide, nucleic acid and mRNA sequences are provided in Table 2BY. Therein, Columns A to C represent a specific suitable construct of the invention derived from NS1 of YFV 17D. The protein designs are indicated in row 1, the specific protein SEQ ID NOs as provided in the sequence listing are in row 2 (“protein”), e.g., for “eSS-NS1-Y” (column A) the protein is SEQ ID NOs: 2201. The SEQ ID NOs of corresponding wild type coding sequences for each protein construct are provided in row 3 (“CDS wt”), e.g., for “eSS-NS1-Y” (column A) the protein is SEQ ID NOs: 2201 and the wt coding sequence is SEQ ID NOs: 2206. The SEQ ID NOs of corresponding codon modified coding sequences (opt1, opt2, opt3, opt4, opt5, opt6, opt11 etc) for each protein construct are provided in row 4 (“CDS opt”), e.g., for “eSS-NS1-Y” (column A) the protein is SEQ ID NOs: 2201 and the wt coding sequence is SEQ ID NOs: 2206, and the codon modified coding sequences are SEQ ID NOs: 2211, 2216, 2221, 2226, 2231, 2236, 2241. Further information e.g. regarding the type of codon modified coding sequence (opt1, opt2, opt3, opt4, opt5, opt6, opt11 etc.) is provided in the <223> identifier of the respective SEQ ID NO in the sequence listing. The SEQ ID NOs of corresponding mRNA constructs comprising said coding sequences comprising suitable 5′-UTRs and 3′-UTRs according to the invention are provided in rows 5 to 16 (row 5: “mRNA UTR a-1”; row 6: “mRNA UTR a-2”; row 7: “mRNA UTR b-4”; row 8: “mRNA UTR c-5”; row 9: “mRNA UTR d-1”; row 10: “mRNA UTR d-5”; row 11: “mRNA UTR g-4”; row 12: “mRNA UTR h-4”; row 13: “mRNA UTR h-5”; row 14: “mRNA UTR i-2”; row 15: “mRNA UTR i-3” row 16 “mRNA UTR a-4”). For example, for “eSS-NS1-Y” (column A) the protein has an amino acid sequence according to SEQ ID NOs: 2201 and the wt coding sequence has a nucleic acid sequence according to SEQ ID NOs: 2206, and the codon modified coding sequences has a nucleic acid sequence according to SEQ ID NOs: 2211, 2216, 2221, 2226, 2231, 2236, 2241, and the mRNA sequences with e.g. UTR a-1 combination has an RNA sequence according to SEQ ID NOs: 2246, 2249, 2252, 2255, 2258, 2261, 2264, 2267, 2558, 2576, 2564, 2582, 2570, 2588. Further information e.g. regarding the type of coding sequence (wt, opt1, opt2, opt3, opt4, opt5, opt6, opt11 etc.) comprised in the mRNA constructs is provided in the <223> identifier of the respective SEQ ID NO in the sequence listing.

TABLE 2BY
mRNA constructs encoding YFV NS1

| | | A | B | C |
|---|---|---|---|---|
| 1 | | eSS-NS1-Y | eSS-NS1 | SSIgE-NS1 |
| 2 | Protein | 2201 | 2202 | 2205 |
| 3 | CDS wt | 2206 | 2207 | 2210 |
| 4 | CDS opt | 2211, 2216, 2221, 2226, | 2212, 2217, 2222, 2227, | 2215, 2220, 2225, 2230, |
| | | 2231, 2236, 2241 | 2232, 2237, 2242 | 2235, 2240, 2245 |
| 5 | mRNA UTR | 2246, 2249, 2252, 2255, | 2247, 2250, 2253, 2256, | 2248, 2251, 2254, 2257, |
| | a-1 | 2258, 2261, 2264, 2267, | 2259, 2262, 2265, 2268, | 2260, 2263, 2266, 2269, |
| | | 2558, 2576, 2564, 2582, | 2559, 2577, 2565, 2583, | 2560, 2578, 2566, 2584, |
| | | 2570, 2588 | 2571, 2589 | 2572, 2590 |
| 6 | mRNA UTR | 2270, 2273, 2276, 2279, | 2271, 2274, 2277, 2280, | 2272, 2275, 2278, 2281, |
| | a-2 | 2282, 2285, 2288, 2291 | 2283, 2286, 2289, 2292 | 2284, 2287, 2290, 2293 |
| 7 | mRNA UTR | 2294, 2297, 2300, 2303, | 2295, 2298, 2301, 2304, | 2296, 2299, 2302, 2305, |
| | b-4 | 2306, 2309, 2312, 2315 | 2307, 2310, 2313, 2316 | 2308, 2311, 2314, 2317 |
| 8 | mRNA UTR | 2318, 2321, 2324, 2327, | 2319, 2322, 2325, 2328, | 2320, 2323, 2326, 2329, |
| | c-5 | 2330, 2333, 2336, 2339 | 2331, 2334, 2337, 2340 | 2332, 2335, 2338, 2341 |
| 9 | mRNA UTR | 2342, 2345, 2348, 2351, | 2343, 2346, 2349, 2352, | 2344, 2347, 2350, 2353, |
| | d-1 | 2354, 2357, 2360, 2363 | 2355, 2358, 2361, 2364 | 2356, 2359, 2362, 2365 |
| 10 | mRNA UTR | 2366, 2369, 2372, 2375, | 2367, 2370, 2373, 2376, | 2368, 2371, 2374, 2377, |
| | d-5 | 2378, 2381, 2384, 2387 | 2379, 2382, 2385, 2388 | 2380, 2383, 2386, 2389 |
| 11 | mRNA UTR | 2390, 2393, 2396, 2399, | 2391, 2394, 2397, 2400, | 2392, 2395, 2398, 2401, |
| | g-4 | 2402, 2405, 2408, 2411 | 2403, 2406, 2409, 2412 | 2404, 2407, 2410, 2413 |
| 12 | mRNA UTR | 2414, 2417, 2420, 2423, | 2415, 2418, 2421, 2424, | 2416, 2419, 2422, 2425, |
| | h-4 | 2426, 2429, 2432, 2435 | 2427, 2430, 2433, 2436 | 2428, 2431, 2434, 2437 |
| 13 | mRNA UTR | 2438, 2441, 2444, 2447, | 2439, 2442, 2445, 2448, | 2440, 2443, 2446, 2449, |
| | h-5 | 2450, 2453, 2456, 2459 | 2451, 2454, 2457, 2460 | 2452, 2455, 2458, 2461 |
| 14 | mRNA UTR | 2462, 2465, 2468, 2471, | 2463, 2466, 2469, 2472, | 2464, 2467, 2470, 2473, |
| | i-2 | 2474, 2477, 2480, 2483, | 2475, 2478, 2481, 2484, | 2476, 2479, 2482, 2485, |
| | | 2486, 2489, 2492, 2495, | 2487, 2490, 2493, 2496, | 2488, 2491, 2494, 2497, |
| | | 2498, 2501, 2504, 2507 | 2499, 2502, 2505, 2508 | 2500, 2503, 2506, 2509 |
| 15 | mRNA UTR | 2510, 2513, 2516, 2519, | 2511, 2514, 2517, 2520, | 2512, 2515, 2518, 2521, |
| | i-3 | 2522, 2525, 2528, 2531, | 2523, 2526, 2529, 2532, | 2524, 2527, 2530, 2533, |
| | | 2561, 2579, 2567, 2585, | 2562, 2580, 2568, 2586, | 2563, 2581, 2569, 2587, |
| | | 2573, 2591 | 2574, 2592 | 2575, 2593 |
| 16 | mRNA UTR | 2534, 2537, 2540, 2543, | 2535, 2538, 2541, 2544, | 2536, 2539, 2542, 2545, |
| | a-4 | 2546, 2549, 2552, 2555 | 2547, 2550, 2553, 2556 | 2548, 2551, 2554, 2557 |

In further preferred embodiments, the artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2246-2593 or a fragment or variant of any of these sequences

In particularly preferred embodiments, the artificial RNA comprises

(a) at least one heterologous 5′ untranslated region (5′-UTR) and/or at least one heterologous 3′ untranslated region (3′-UTR) and
(b) at least one coding sequence operably linked to said 3′-UTR and/or 5′-UTR encoding at least one antigenic peptide or protein derived from a YFV NS1 polyprotein or a fragment or variant thereof, wherein
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2246-2269, 2558-2560, 2576-2578, 2564-2566, 2582-2584, 2570-2572, 2588-2590 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2534-2557 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2294-2317 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2510-2533, 2561-2563, 2579-2581, 2567-2569, 2585-2587, 2573-2575, 2591-2593 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2318-2341 or a fragment or variant of any of these sequences; or
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 2390-2413 or a fragment or variant of any of these sequences.

### Composition:

A second aspect relates to a composition comprising at least one artificial RNA of the first aspect.

In a preferred embodiment of the second aspect, the composition comprises at least one artificial RNA of the first aspect and, optionally, at least one pharmaceutically acceptable carrier.

The term “pharmaceutically acceptable carrier” as used herein preferably includes the liquid or non-liquid basis of the composition. If the composition is provided in liquid form, the carrier will preferably be water, typically pyrogen-free water; isotonic saline or buffered (aqueous) solutions, e.g. phosphate, citrate etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to a preferred embodiment, the sodium, calcium and, optionally, potassium salts may occur in the form of their halogenides, e.g. chlorides, iodides, or bromides, in the form of their hydroxides, carbonates, hydrogen carbonates, or sulfates, etc. Without being limited thereto, examples of sodium salts include e.g. NaCl, NaI, NaBr, Na₂CO₃, NaHCO₃, Na₂SO₄, examples of the optional potassium salts include e.g. KCl, KI, KBr, K₂CO₃, KHCO₃, K₂SO₄, and examples of calcium salts include e.g. CaCl₂), Cal₂, CaBr₂, CaCO₃, CaSO₄, Ca(OH)₂. Furthermore, organic anions of the aforementioned cations may be contained in the buffer.

Furthermore, one or more compatible solid or liquid fillers or diluents or encapsulating compounds may be used as well, which are suitable for administration to a person. The term “compatible” as used herein means that the constituents of the composition are capable of being mixed with the at least one RNA of the composition, in such a manner that no interaction occurs, which would substantially reduce the biological activity or the pharmaceutical effectiveness of the composition under typical use conditions. Pharmaceutically acceptable carriers, fillers and diluents must have sufficiently high purity and sufficiently low toxicity to make them suitable for administration to a person to be treated. Compounds which may be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

Further additives, which may be included in the composition are emulsifiers, such as, for example, Tween; wetting agents, such as, for example, sodium lauryl sulfate; colouring agents; taste-imparting agents, pharmaceutical carriers; tablet-forming agents; stabilizers; antioxidants; preservatives.

In embodiments, the composition as defined herein may comprise a plurality or at least more than one of the artificial RNAs as defined in the context of the first aspect of the invention.

In embodiments, the composition as defined herein may comprise a plurality or at least more than one of the artificial RNAs as defined in the context of the first aspect of the invention and at least one further artificial RNAs.

Notably, embodiments relating to the artificial RNA of the first aspect may likewise be read on and be understood as suitable embodiments of the at least one further artificial RNA of the second aspect (e.g., embodiments relating to UTR combinations, cds optimizations, histone stem loop, PolyA, PolyC, cap structure, mRNA structure, mRNA production and mRNA purification etc.).

In various embodiments, the at least one artificial RNA of the first aspect and the at least one further artificial RNA as specified herein is derived from different YF viruses.

In preferred embodiments, the at least one artificial RNA of the first aspect and the at least one further artificial RNA as specified herein is derived from the same YF virus.

In preferred embodiments, the at least one artificial RNA of the first aspect is derived from YF 17D and the at least one further artificial RNA as specified herein is derived from YF 17D.

In embodiments, the at least one RNA comprised in the composition is a bi- or multicistronic nucleic acid, particularly a bi- or multicistronic nucleic acid as defined herein, which encodes the at least two, three, four, five, six, seven, eight, nine, ten, eleven or twelve distinct antigenic peptides or protein derived from the same YFV and/or a different YFV.

In embodiment, the composition may comprise at least 2, 3, 4, 5, 6, 7, 8, 9, 10 or even more different artificial RNAs as defined in the context of the first aspect of the invention each encoding at least one antigenic peptide or protein derived from genetically the same YFV or a fragment or variant thereof. The terms “same” or “same YFV” as used in the context of a virus, e.g. “same virus”, have to be understood as genetically the same. Particularly, said (genetically) same virus expresses the same proteins or peptides, wherein all proteins or peptides have the same amino acid sequence. Particularly, said (genetically) same YFV express essentially the same proteins, peptides or polyproteins, wherein these protein, peptide or polyproteins preferably do not differ in their amino acid sequence(s).

In embodiments, the composition comprises at least 2, 3, 4, 5, 6, 7, 8, 9, 10 or even more different RNAs as defined in the context of the first aspect of the invention each encoding at least one peptide or protein derived from a genetically different YFV or a fragment or variant thereof. The terms “different” or “different YFV” as used throughout the present specification in the context of a virus, e.g. “different” virus, has to be understood as the difference between at least two respective viruses, wherein the difference is manifested on the RNA genome of the respective different virus. Particularly, said (genetically) different YFV express at least one different protein, peptide or polyprotein, wherein the at least one different protein, peptide or polyprotein preferably differs in at least one amino acid.

In particularly preferred embodiments, the composition comprises at least one YFV prME RNA constructs as defined in the context of the first aspect of the invention (preferably any one selected from SEQ ID NOs: 528-1320, 1609-2200) and, in addition, at least 1, 2, 3, 4, 5, 6, 7 or 8 YFV RNA constructs encoding another YFV protein (e.g. NS1, NS2), preferably a YFV RNA constructs comprising a nucleic acid sequence selected from SEQ ID NOs: 57-540, 587-954 of patent application EP17207141.7 and/or a nucleic acid sequence comprising a coding sequence encoding an amino acid sequence selected from SEQ ID NOs: 23-56, 541-586 of patent application EP17207141.7.

In a preferred embodiment in that context, the composition comprises one YFV prME RNA constructs as defined herein in the context of the first aspect and, in addition, one YFV NS1 RNA construct as defined in the context of the first aspect. In a preferred embodiment in that context, the composition comprises one YFV prME RNA construct as defined herein and, in addition, on YFV NS2 RNA construct. In a preferred embodiment in that context, the composition comprises one YFV prME RNA constructs as defined herein and, in addition, on YFV NS3 RNA construct. In a preferred embodiment in that context, the composition comprises one YFV prME RNA constructs as defined herein and, in addition, on YFV NS4 RNA construct. In a preferred embodiment in that context, the composition comprises one YFV prME RNA constructs as defined herein and, in addition, on YFV NS5 RNA construct

In a particularly preferred embodiment in that context, the composition comprises at least one YFV prME RNA construct (as defined in the first aspect) and, in addition, at least one YFV NS1 RNA construct (as defined in the first aspect). The combination of prME and NS1 is advantageous, as NS1 may suitably reduce pathogenesis trough decrease of vascular leakage.

In a particularly preferred embodiment the composition comprises at least one YFV prME RNA construct, preferably selected from X-SS-prME-XX, X-SS-prME, SS-prME, SSjev(V3)-prME-XX, X-SS-prMEdelstem_TM-JEV or SSjev(V3)-prMEdelstem_TM-JEV and, in addition, at least one YFV NS1 RNA construct, preferably selected from eSS-NS1-Y, eSS-NSE and SSIgE-NS1.

In various embodiments, different combinations of prME RNA constructs and NS1 RNA constructs are suitably comprised in the composition (as disclosed in Table 2C; combinations 1-18):

TABLE 2C
suitable combinations of prME and NS1 RNA constructs

| | | SEQ | | SEQ |
|---|---|---|---|---|
| Combi- | prME | ID NO: | NS1 | ID NO: |
| nation | Construct | Protein | Construct | Protein |
| 1 | X-SS-prME-XX | 121 | eSS-NS1-Y | 2201 |
| 2 | X-SS-prME-XX | 121 | eSS-NS1 | 2202 |
| 3 | X-SS-prME-XX | 121 | SSIgE-NS1 | 2205 |
| 4 | X-SS-prME | 122 | eSS-NS1-Y | 2201 |
| 5 | X-SS-prME | 122 | eSS-NS1 | 2202 |
| 6 | X-SS-prME | 122 | SSIgE-NS1 | 2205 |
| 7 | SS-prME | 126 | eSS-NS1-Y | 2201 |
| 8 | SS-prME | 126 | eSS-NS1 | 2202 |
| 9 | SS-prME | 126 | SSIgE-NS1 | 2205 |
| 10 | SSjev(V3)-prME-XX | 142 | eSS-NS1-Y | 2201 |
| 11 | SSjev(V3)-prME-XX | 142 | eSS-NS1 | 2202 |
| 12 | SSjev(V3)-prME-XX | 142 | SSIgE-NS1 | 2205 |
| 13 | X-SS-prMEdelstem_TM-JEV | 1587 | eSS-NS1-Y | 2201 |
| 14 | X-SS-prMEdelstem_TM-JEV | 1587 | eSS-NS1 | 2202 |
| 15 | X-SS-prMEdelstem_TM-JEV | 1587 | SSIgE-NS1 | 2205 |
| 16 | SSjev(V3)- | 1588 | eSS-NS1-Y | 2201 |
| | prMEdelstem_TM-JEV | | | |
| 17 | SSjev(V3)- | 1588 | eSS-NS1 | 2202 |
| | prMEdelstem_TM-JEV | | | |
| 18 | SSjev(V3)- | 1588 | SSIgE-NS1 | 2205 |
| | prMEdelstem_TM-JEV | | | |

In a particularly preferred embodiment, the composition comprises one YFV prME RNA construct and, in addition, one YFV NS1 RNA construct, preferably eSS-NS1 or eSS-NS1-Y.

In a particularly preferred embodiment, the composition comprises one YFV prME RNA construct and, in addition, one YFV NS1 RNA construct, preferably SSIgE-NS1-Y.

Accordingly, in preferred embodiments the composition of the second aspect may suitably comprise at least one artificial RNA encoding YF prME (as defined in the first aspect) and at least one artificial RNA comprising at least one coding sequence encoding at least one antigenic peptide or protein derived from YF NS1 (as defined in the first aspect)

In particularly preferred embodiments, one RNA construct encoding NS1, selected from SEQ ID NOs: 2579, 2588, 2580, 2589, 2581, 2590, and one RNA construct encoding prME, selected from SEQ ID NOs: 1194, 534, 2171, 2190, 2183, 2173, 2191, 1195, 2174, 2192, 2175, 2193, 2176, 2194, are comprised in the composition of the invention.

In a preferred embodiment, one RNA construct encoding NS1, SEQ ID NOs: 2579, and one RNA construct encoding prME, selected from SEQ ID NOs: 1194, 534, 2171, 2190, 2183, 2173, 2191, 1195, 2174, 2192, 2175, 2193, 2176, 2194, are comprised in the composition of the invention.

In a preferred embodiment, one RNA construct encoding NS1, SEQ ID NOs: 2588, and one RNA construct encoding prME, selected from SEQ ID NOs: 1194, 534, 2171, 2190, 2183, 2173, 2191, 1195, 2174, 2192, 2175, 2193, 2176, 2194, are comprised in the composition of the invention.

In a preferred embodiment, one RNA construct encoding NS1, SEQ ID NOs: 2580, and one RNA construct encoding prME, selected from SEQ ID NOs: 1194, 534, 2171, 2190, 2183, 2173, 2191, 1195, 2174, 2192, 2175, 2193, 2176, 2194, are comprised in the composition of the invention.

In a preferred embodiment, one RNA construct encoding NS1, SEQ ID NOs: 2589, and one RNA construct encoding prME, selected from SEQ ID NOs: 1194, 534, 2171, 2190, 2183, 2173, 2191, 1195, 2174, 2192, 2175, 2193, 2176, 2194, are comprised in the composition of the invention.

In a preferred embodiment, one RNA construct encoding NS1, SEQ ID NOs: 2581, and one RNA construct encoding prME, selected from SEQ ID NOs: 1194, 534, 2171, 2190, 2183, 2173, 2191, 1195, 2174, 2192, 2175, 2193, 2176, 2194, are comprised in the composition of the invention.

In a preferred embodiment, one RNA construct encoding NS1, SEQ ID NOs: 2590, and one RNA construct encoding prME, selected from SEQ ID NOs: 1194, 534, 2171, 2190, 2183, 2173, 2191, 1195, 2174, 2192, 2175, 2193, 2176, 2194, are comprised in the composition of the invention.

It has to be understood that in the context of the invention, certain combinations of coding sequences may be generated by any combination of monocistronic, bicistronic and multicistronic artificial nucleic acids and/or multi-antigen-constructs/nucleic acid to obtain a nucleic acid composition encoding multiple antigenic peptides or proteins as defined herein.

Furthermore, one or more compatible solid or liquid fillers or diluents or encapsulating compounds may be used as well, which are suitable for administration to a person. The term “compatible” as used herein means that the constituents of the composition are capable of being mixed with the at least one RNA and, optionally, the further artificial RNA of the composition, in such a manner that no interaction occurs, which would substantially reduce the biological activity or the pharmaceutical effectiveness of the composition under typical use conditions. Pharmaceutically acceptable carriers, fillers and diluents must have sufficiently high purity and sufficiently low toxicity to make them suitable for administration to a person to be treated. Compounds which may be used as pharmaceutically acceptable carriers, fillers or constituents thereof are sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

Further additives, which may be included in the composition are emulsifiers, such as, for example, Tween;

wetting agents, such as, for example, sodium lauryl sulfate; colouring agents; taste-imparting agents, pharmaceutical carriers; tablet-forming agents; stabilizers; antioxidants; preservatives.

In a preferred embodiment of the second aspect, the at least one artificial RNA of the first aspect is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

The term “cationic or polycationic compound” as used herein will be recognized and understood by the person of ordinary skill in the art, and are for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

Cationic or polycationic compounds, being particularly preferred in this context may be selected from the following list of cationic or polycationic peptides or proteins of fragments thereof: protamine, nucleoline, spermine or spermidine, or other cationic peptides or proteins, such as poly-L-lysine (PLL), poly-arginine, basic polypeptides, cell penetrating peptides (CPPB), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides, pAntp, 051, FGF, Lactoferrin, Transportan, Buforin-2, Bac715-24, SynB, SynB(1), pVEC, hCT-derived peptides, SAP, or histones. More preferably, the nucleic acid as defined herein, preferably the mRNA as defined herein, is complexed with one or more polycations, preferably with protamine or oligofectamine, most preferably with protamine.

In a preferred embodiment of the second aspect, the at least one artificial RNA of the first aspect is complexed with protamine.

Further preferred cationic or polycationic compounds, which can be used as transfection or complexation agent may include cationic polysaccharides, for example chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-aminoacid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycol); etc.

In this context it is particularly preferred that the at least one artificial RNA as defined herein is complexed or at least partially complexed with a cationic or polycationic compound and/or a polymeric carrier, preferably cationic proteins or peptides. In this context, the disclosure of WO2010/037539 and WO2012/113513 is incorporated herewith by reference. Partially means that only a part of the artificial nucleic acid is complexed with a cationic compound and that the rest of the artificial nucleic acid is (comprised in the inventive (pharmaceutical) composition) in uncomplexed form (“free”).

In a preferred embodiment of the second aspect, the at least one artificial RNA of the first aspect is complexed with one or more cationic or polycationic compounds, preferably protamine, and at least one free artificial RNA of the first aspect.

In this context it is particularly preferred that the at least one artificial RNA as defined herein, is complexed, or at least partially complexed with protamine. Preferably, the molar ratio of the nucleic acid, particularly the RNA of the protamine-complexed RNA to the free RNA may be selected from a molar ratio of about 0.001:1 to about 1:0.001, including a ratio of about 1:1. Suitably, the complexed RNA is complexed with protamine by addition of protamine-trehalose solution to the RNA sample at a RNA:protamine weight to weight ratio (w/w) of 2:1.

Further preferred cationic or polycationic proteins or peptides that may be used for complexation can be derived from Formula (Arg)l; (Lys)m; (His)n; (Orn)o; (Xaa)x of the patent application WO2009/030481 or WO2011/026641, the disclosure of WO2009/030481 or WO2011/026641 relating thereto incorporated herewith by reference.

In a preferred embodiment of the second aspect, the at least one artificial RNA of the first aspect is complexed, or at least partially complexed with cationic or polycationic proteins or peptides preferably selected from CHHHHHHRRRRHHHHHHC (SEQ ID NO: 55), CRRRRRRRRRRRRC (SEQ ID NO: 52), CRRRRRRRRRRRR (SEQ ID NO: 53), or WRRRRRRRRRRRRC (SEQ ID NO: 54), or a fragment or variant of any of these sequences.

According to embodiments, the composition of the present invention comprises the RNA as defined herein, and a polymeric carrier.

The term “polymeric carrier” as used herein will be recognized and understood by the person of ordinary skill in the art, and are for example intended to refer to a compound that facilitates transport and/or complexation of another compound (cargo). A polymeric carrier is typically a carrier that is formed of a polymer. A polymeric carrier may be associated to its cargo (nucleic acid, RNA) by covalent or non-covalent interaction

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used according to the present invention may comprise mixtures of cationic peptides, proteins or polymers and optionally further components as defined herein, which are crosslinked by disulfide bonds (via —SH groups).

In this context, polymeric carriers according to Formula {(Arg)l; (Lys)m; (His)n; (Orn)o; (Xaa′)x(Cys)y} and Formula Cys,{(Arg)l; (Lys)m; (His)n; (Orn)o; (Xaa)x}Cys₂ of the patent application WO2012/013326 are preferred, the disclosure of WO2012/013326 relating thereto incorporated herewith by reference.

In embodiments, the polymeric carrier used to complex the RNA as defined herein may be derived from a polymeric carrier molecule according Formula (L-P¹-S-[S-P²-S]ₙ-S-P³-L) of the patent application WO2011/026641, the disclosure of WO2011/026641 relating thereto incorporated herewith by reference.

In embodiments, the polymeric carrier compound is formed by, or comprises or consists of the peptide elements CysArg12Cys (SEQ ID NO: 52) or CysArg12 (SEQ ID NO: 53) or TrpArg12Cys (SEQ ID NO: 54). In particularly preferred embodiments, the polymeric carrier compound consists of a (R₁₂C)-(R₁₂C) dimer, a (WR₁₂C)—(WR₁₂C) dimer, or a (CR₁₂)—(CR₁₂C)—(CR₁₂) trimer, wherein the individual peptide elements in the dimer (e.g. (WR₁₂C)), or the trimer (e.g. (CR₁₂)), are connected via —SH groups.

In a preferred embodiment of the second aspect, the at least one artificial RNA of the first aspect is complexed or associated with a polyethylene glycol/peptide polymer comprising HO-PEG5000-S-(S-CHHHHHHRRRRHHHHHHC-S-)7-S-PEG5000-OH (SEQ ID NO: 55 as peptide monomer).

In a further preferred embodiment of the second aspect, the at least one artificial RNA of the first aspect is complexed or associated with a polyethylene glycol/peptide polymer comprising HO-PEG5000-S-(S-CHHHHHHRRRRHHHHHHC-S-)4-S-PEG5000-OH (SEQ ID NO: 55 as peptide monomer).

In a further preferred embodiment of the second aspect, the at least one artificial RNA of the first aspect is complexed or associated with a polyethylene glycol/peptide polymer comprising HO-PEG5000-S-(S-CGHHHHHRRRRHHHHHGC-S-)7-S-PEG5000-OH (SEQ ID NO: 1321 as peptide monomer).

In a further preferred embodiment of the second aspect, the at least one artificial RNA of the first aspect is complexed or associated with a polyethylene glycol/peptide polymer comprising HO-PEG5000-S-(S-CGHHHHHRRRRHHHHHGC-S-)4-S-PEG5000-OH (SEQ ID NO: 1321 as peptide monomer).

In other embodiments, the composition comprises at least one artificial RNA as described herein, wherein the at least one artificial RNA is complexed or associated with polymeric carriers and, optionally, with at least one lipid component as described in the published PCT applications WO2017/212008A1, WO2017/212006A1, WO2017/212007A1, and WO2017/212009A1. In this context, the disclosures of WO2017/212008A1, WO2017/212006A1, WO2017/212007A1, and WO2017/212009A1 are herewith incorporated by reference.

In a particularly preferred embodiment, the polymeric carrier is a peptide polymer, preferably a polyethylene glycol/peptide polymer as defined above, and a lipid component, preferably a lipidoid component, more preferably lipidoid component.

In preferred embodiment of the second aspect, the at least one artificial RNA of the first aspect is complexed or associated with a polymeric carrier, preferably with a polyethylene glycol/peptide polymer as defined above, and a lipidoid component, wherein the lipidoid component is a compound according to Formula A

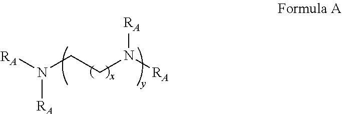

wherein
R_{A} is independently selected for each occurrence an unsubstituted, cyclic or acyclic, branched or unbranched C₁₋₂₀ aliphatic group; a substituted or unsubstituted, cyclic or acyclic, branched or unbranched C₁₋₂₅ heteroaliphatic group; a substituted or unsubstituted aryl; a substituted or unsubstituted heteroaryl;

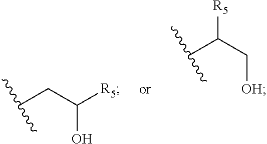

wherein at least one R_{A} is

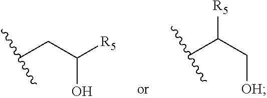

R₅ is independently selected for each occurrence of from an unsubstituted, cyclic or acyclic, branched or unbranched C₈₋₁₆ aliphatic; a substituted or unsubstituted aryl; or a substituted or unsubstituted heteroaryl;
each occurrence of x is an integer from 1 to 10;
each occurrence of y is an integer from 1 to 10;
   or a pharmaceutically acceptable salt thereof.

In a preferred embodiment, the lipidoid component is 3-C12-OH according to Formula B

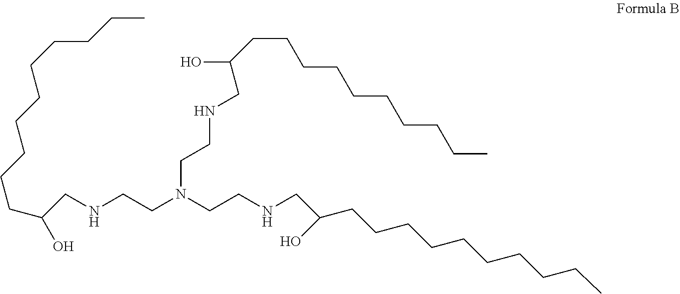 [SMILES: CCCCCCCCCCC(O)CNCCN(CCNCC(O)CCCCCCCCCC)CCNCC(O)CCCCCCCCCC]

In preferred embodiments, the peptide polymer comprising lipidoid 3-C12-OH as specified above is used to complex the RNA of the first aspect to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the artificial nucleic acid. In that context, the disclosure of WO2017/212009A1, in particular claims 1 to 10 of WO2017/212009A1, and the specific disclosure relating thereto is herewith incorporated by reference.

### Encapsulation/Complexation in LNPs:

In preferred embodiments of the second aspect, the artificial RNA of the first aspect is complexed or associated with one or more lipids (e.g. cationic lipids and/or neutral lipids), thereby forming liposomes, lipid nanoparticles (LNPs), lipoplexes, and/or nanoliposomes.

For compositions comprising more than one artificial RNA construct as defined herein (prME and NS1), said constructs may be co-formulated in e.g. LNPs to form the respective composition.

Alternatively, said more than one artificial RNA construct may be formulated separately, and may subsequently be combined, to form the respective composition.

In this context, the terms “complexed” or “associated” refer to the essentially stable combination of artificial RNA of the first aspect with one or more lipids into larger complexes or assemblies without covalent binding.

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of RNA. For example, a liposome, a lipid complex, a lipoplex and the like are within the scope of a lipid nanoparticle (LNP).

Accordingly, in preferred embodiments of the second aspect, the artificial RNA of the first aspect is complexed with one or more lipids thereby forming lipid nanoparticles (LNP).

LNPs typically comprise a cationic lipid and one or more excipient selected from neutral lipids, charged lipids, steroids and polymer conjugated lipids (e.g. PEGylated lipid). The RNA may be encapsulated in the lipid portion of the LNP or an aqueous space enveloped by some or the entire lipid portion of the LNP. The RNA or a portion thereof may also be associated and complexed with the LNP. An LNP may comprise any lipid capable of forming a particle to which the nucleic acids are attached, or in which the one or more nucleic acids are encapsulated. Preferably, the LNP comprising nucleic acids comprises one or more cationic lipids, and one or more stabilizing lipids. Stabilizing lipids include neutral lipids and PEGylated lipids.

The cationic lipid of an LNP may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

The LNP may comprise any further cationic or cationisable lipid, i.e. any of a number of lipid species which carry a net positive charge at a selective pH, such as physiological pH.

Such lipids include, but are not limited to, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyI)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DM A), 2,2-Dilinoleyl-4-dimethylaminomethyl[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)[1,3]-dioxolane (DLin-K-C2-DMA), 2,2-dilinoleyl-4-dimethylaminomethyl[1,3]-dioxolane (DLin-K-DMA), (6Z,9Z,28Z,31Z)-heptatriaconta-6,9,28,31-tetraen-19-yl4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing.

In some embodiments, the lipid is selected from the group consisting of 98N12-5, C12-200, and ckk-E12.

In one embodiment, the further cationic lipid is an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In one embodiment, the artificial RNA of the first aspect may be formulated in an aminoalcohol lipidoid. Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017/075531A1, hereby incorporated by reference.

In another embodiment, ionizable lipids can also be the compounds as disclosed in WO2015/074085A1 (i.e. ATX-001 to ATX-032 or the compounds as specified in claims 1-26), U.S. Appl. No. 61/905,724 and Ser. No. 15/614,499 or U.S. Pat. Nos. 9,593,077 and 9,567,296 hereby incorporated by reference in their entirety.

In that context, any lipid derived from generic Formula (X1)

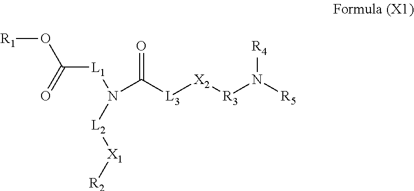

wherein, R1 and R2 are the same or different, each a linear or branched alkyl consisting of 1 to 9 carbons, an alkenyl or alkynyl consisting of 2 to 11 carbons, L1 and L2 are the same or different, each a linear alkylene or alkenylene consisting of 5 to 18 carbons, or forming a heterocycle with N, Xi is a bond, or is —CO—O— whereby -L2-CO—O—R2 is formed, X2 is S or O, L3 is a bond or a linear or branched alkylene consisting of 1 to 6 carbons, or forming a heterocycle with N, R3 is a linear or branched alkylene consisting of 1 to 6 carbons, and R4 and R5 are the same or different, each hydrogen or a linear or branched alkyl consisting of 1 to 6 carbons; or a pharmaceutically acceptable salt thereof may be suitably used.

In other embodiments, suitable cationic lipids can also be the compounds as disclosed in WO2017/117530A1 (i.e. lipids 13, 14, 15, 16, 17, 18, 19, 20, or the compounds as specified in the claims), hereby incorporated by reference in its entirety.

In that context, any lipid derived from generic Formula (X2)

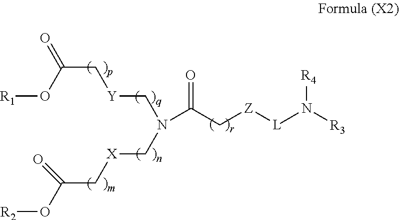

wherein,
X is a linear or branched alkylene or alkenylene, monocyclic, bi cyclic, or tricyclic arene or heteroarene;
Y is a bond, an ethene, or an unsubstituted or substituted aromatic or heteroaromatic ring; Z is S or 0;
L is a linear or branched alkylene of 1 to 6 carbons;
R-3 and R4 are independently a linear or branched alkyl of 1 to 6 carbons;
Ri and R2 are independently a linear or branched alkyl or alkenyl of 1 to 20 carbons; r is 0 to 6; and
m, n, p, and q are independently 1 to 18;
wherein when n=q, m=p, and Ri=R2, then X and Y differ;
wherein when X=Y, n=q, m=p, then Ri and R2 differ;
wherein when X=Y, n=q, and Ri=R2, then m and p differ; and
wherein when X=Y, m=p, and Ri=R2, then n and q differ;
or a pharmaceutically acceptable salt thereof.

In preferred embodiments, a lipid may be used derived from Formula (X2), wherein, X is a bond, linear or branched alkylene, alkenylene, or monocyclic, bicyclic, or tricyclic arene or heteroarene; Y is a monocyclic, bicyclic, or tricyclic arene or heteroarene; Z is S or O; L is a linear or branched alkylene of 1 to 6 carbons; R3 and R4 are independently a linear or branched alkyl of 1 to 6 carbons; Ri and R2 are independently a linear or branched alkyl or alkenyl of 1 to 20 carbons; r is 0 to 6; and m, n, p, and q are independently 1 to 18; or a pharmaceutically acceptable salt thereof may be suitably used.

In preferred embodiments, ionizable lipids may also be selected from the lipid compounds disclosed in PCT application PCT/EP2017/077517 (i.e. lipid compounds derived from Formula I, II, and III of PCT/EP2017/077517, or lipid compounds as specified in claims 1 to 12 of PCT/EP2017/077517), the disclosure of PCT/EP2017/077517 hereby incorporated by reference in its entirety. In that context, lipid compounds disclosed in Table 7 of PCT/EP2017/077517 (e.g. lipid compounds derived from Formula I-1 to 1-41) and lipid compounds disclosed in Table 8 of PCT/EP2017/077517 (e.g. lipid compounds derived from Formula II-1 to II-36) may be suitably used in the context of the invention. Accordingly, Formula I-1 to Formula I-41 and Formula II-1 to Formula II-36 of PCT/EP2017/077517, and the specific disclosure relating thereto, are herewith incorporated by reference.

In particularly preferred embodiments of the second aspect, a suitable lipid may be a cationic lipid according to

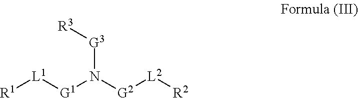

or a pharmaceutically acceptable salt, tautomer, prodrug or stereoisomer thereof, wherein, R1, R2, R3, L1, L2, G1, G2, and G3 are as below.

Formula (III) is further defined in that:

one of L¹ or L² is —O(C═O)—, —(C═O)O—, —C(═O)—, —O—, —S(O)ₓ—, —S—S—, —C(═O)S—, SC(═O)—, —NRaC(═O)—, —C(═O)NR^{a}—, —NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, —NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

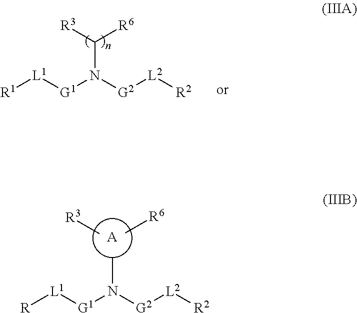

wherein:
A is a 3 to 8-membered cycloalkyl or cycloalkylene ring; R⁶ is, at each occurrence, independently H, OH or C₁-C₂₄ alkyl; n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

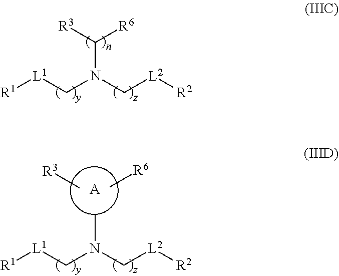

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In preferred embodiments of the second aspect, the cationic lipid of the LNP is a compound of Formula III, wherein:

L¹ and L² are each independently —O(C═O)— or (C═O)—O—;
G³ is C₁-C₂₄ alkylene or C₁-C₂₄ alkenylene; and
R³ is H or OR⁵.

In some different embodiments of Formula (III), the lipid has one of the following structures (IIIE) or (IIIF):

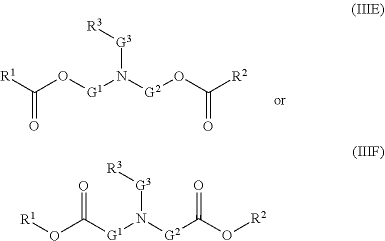

In some of the foregoing embodiments of Formula (III), the lipid has one of the following structures (IIIG), (IIIH), (IIII), or (IIIJ):

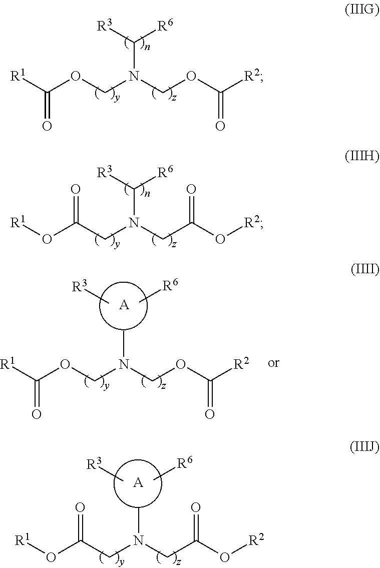

In some of the foregoing embodiments of Formula (III), n is an integer ranging from 2 to 12, for example from 2 to 8 or from 2 to 4. In some embodiments, n is 3, 4, 5 or 6. In some embodiments, n is 3. In some embodiments, n is 4. In some embodiments, n is 5. In some embodiments, n is 6. In some other of the foregoing embodiments of Formula (III), y and z are each independently an integer ranging from 2 to 10. For example, in some embodiments, y and z are each independently an integer ranging from 4 to 9 or from 4 to 6. In some of the foregoing embodiments of Formula (III), R⁶ is H. In other of the foregoing embodiments, R⁶ is C₁-C₂₄ alkyl. In other embodiments, R⁶ is OH. In some embodiments of Formula (III), G³ is unsubstituted. In other embodiments, G3 is substituted. In various different embodiments, G³ is linear C₁-C₂₄ alkylene or linear C₁-C₂₄ alkenylene. In some other foregoing embodiments of Formula (III), R¹ or R², or both, is C₆-C₂₄ alkenyl. For example, in some embodiments, R¹ and R² each, independently have the following structure:

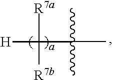

wherein:
R^{7a} and R⁷b are, at each occurrence, independently H or C₁-C₁₂ alkyl; and a is an integer from 2 to 12, wherein R^{7a}, R^{7b} and a are each selected such that R¹ and R² each independently comprise from 6 to 20 carbon atoms. For example, in some embodiments a is an integer ranging from 5 to 9 or from 8 to 12. In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

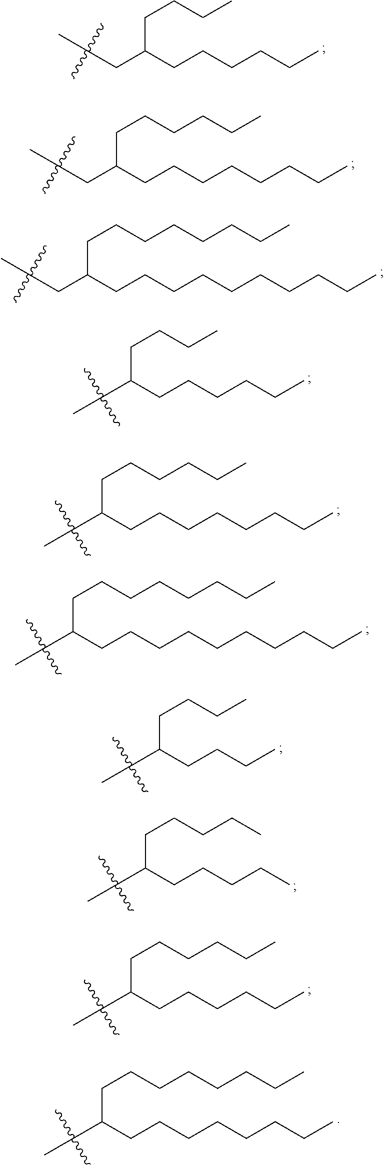

In preferred embodiments of the second aspect, the cationic lipid of the LNP is a compound of Formula III, wherein:

L¹ and L² are each independently —O(C═O)— or (C═O)—O—; and
R¹ and R² each independently have one of the following structures:

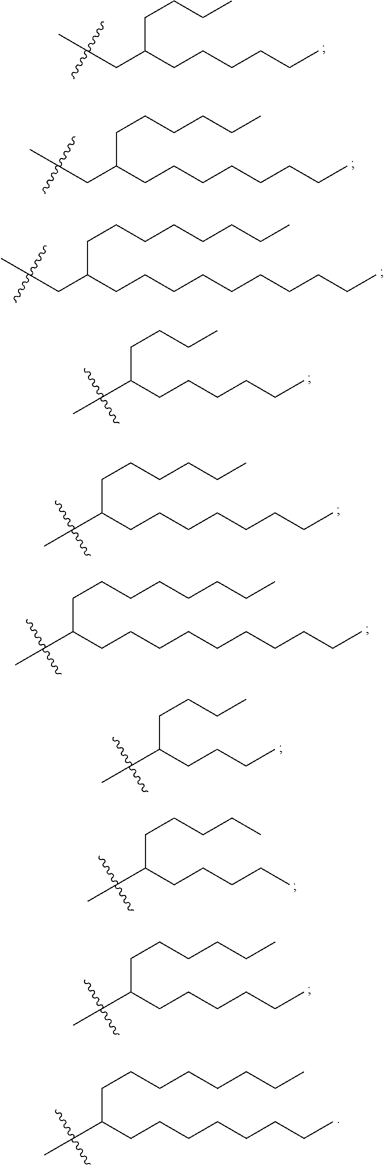

In some of the foregoing embodiments of Formula (III), R³ is OH, CN, —C(═O)OR⁴, —OC(═O)R⁴ or —NHC(═O)R⁴. In some embodiments, R⁴ is methyl or ethyl.

In preferred embodiments of the second aspect, the cationic lipid of the LNP is a compound of Formula III, wherein R³ is OH.

In particularly preferred embodiment of the second aspect, the artificial RNA of the first aspect is complexed with one or more lipids thereby forming lipid nanoparticles (LNP), wherein the LNP is selected from structures III-1 to III-36 (see Table A).

TABLE A
Representative Compounds of Formula (III)

| No. | Structure |
|---|---|
| III-1 | 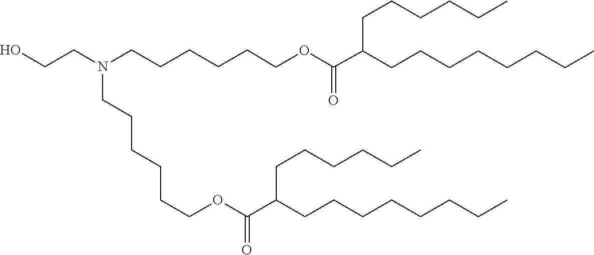 [SMILES: CCCCCCCCC(CCCCCC)C(=O)OCCCCCCN(CCO)CCCCCCOC(=O)C(CCCCCC)CCCCCCCC] |
| III-2 | 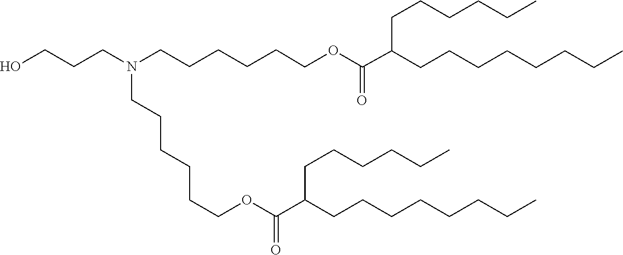 [SMILES: CCCCCCCCC(CCCCCC)C(=O)OCCCCCCN(CCCO)CCCCCCOC(=O)C(CCCCCC)CCCCCCCC] |
| III-3 | 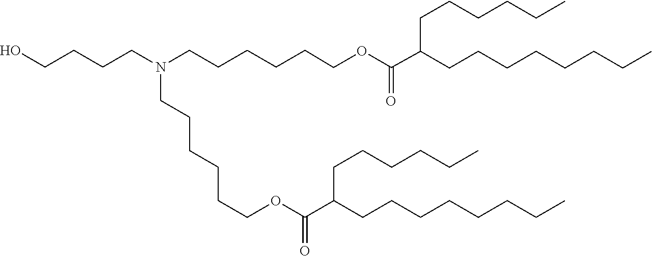 [SMILES: CCCCCCCCC(CCCCCC)C(=O)OCCCCCCN(CCCCO)CCCCCCOC(=O)C(CCCCCC)CCCCCCCC] |
| III-4 | 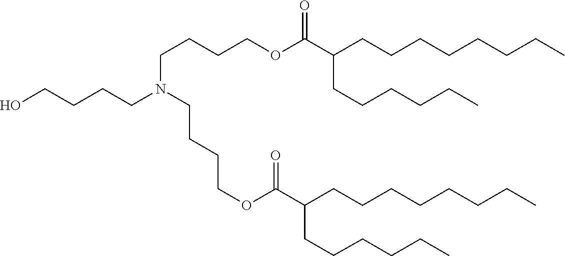 [SMILES: CCCCCCCCC(CCCCCC)C(=O)OCCCCN(CCCCO)CCCCOC(=O)C(CCCCCC)CCCCCCCC] |
| III-5 | 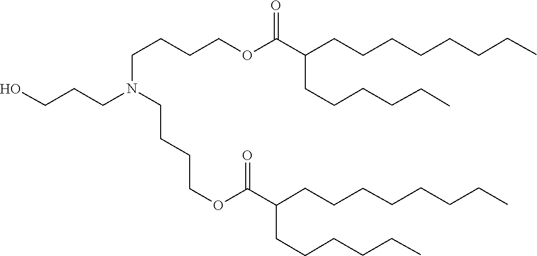 [SMILES: CCCCCCCCC(CCCCCC)C(=O)OCCCCN(CCCO)CCCCOC(=O)C(CCCCCC)CCCCCCCC] |
| III-6 | 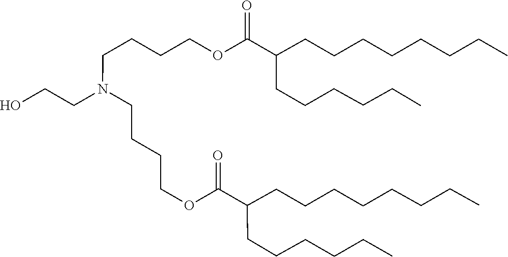 [SMILES: CCCCCCCCC(CCCCCC)C(=O)OCCCCN(CCO)CCCCOC(=O)C(CCCCCC)CCCCCCCC] |
| III-7 | 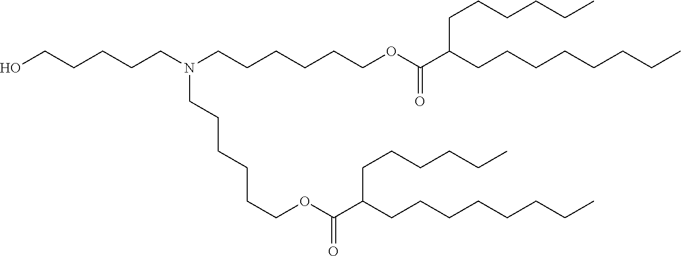 [SMILES: CCCCCCCCC(CCCCCC)C(=O)OCCCCCCN(CCCCCO)CCCCCCOC(=O)C(CCCCCC)CCCCCCCC] |
| III-8 | 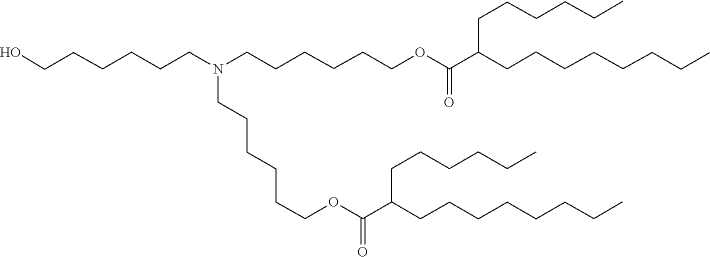 [SMILES: CCCCCCCCC(CCCCCC)C(=O)OCCCCCCN(CCCCCCO)CCCCCCOC(=O)C(CCCCCC)CCCCCCCC] |
| III-9 | 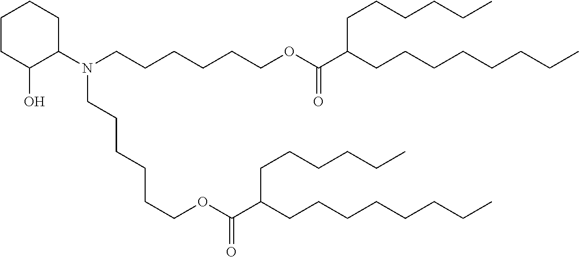 [SMILES: CCCCCCCCC(CCCCCC)C(=O)OCCCCCCN(CCCCCCOC(=O)C(CCCCCC)CCCCCCCC)C1CCCCC1O] |
| III-10 | 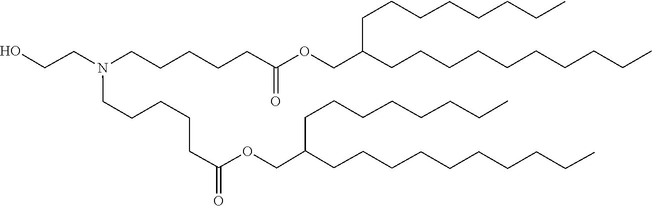 [SMILES: CCCCCCCCCCC(CCCCCCCC)COC(=O)CCCCCN(CCO)CCCCCC(=O)OCC(CCCCCCCC)CCCCCCCCCC] |
| III-11 | 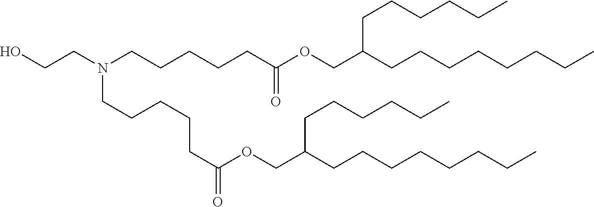 [SMILES: CCCCCCCCC(CCCCCC)COC(=O)CCCCCN(CCO)CCCCCC(=O)OCC(CCCCCC)CCCCCCCC] |
| III-12 | 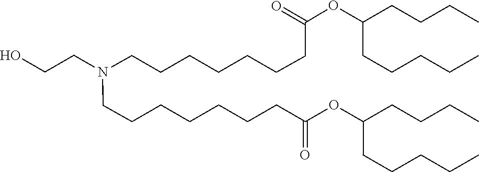 [SMILES: CCCCCC(CCCC)OC(=O)CCCCCCCN(CCO)CCCCCCCC(=O)OC(CCCC)CCCCC] |
| III-13 | 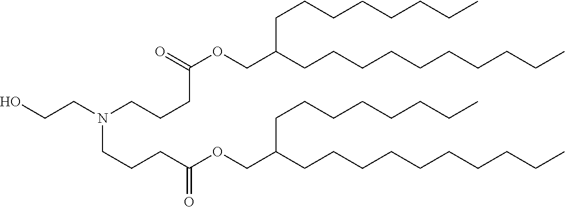 [SMILES: CCCCCCCCCCC(CCCCCCCC)COC(=O)CCCN(CCO)CCCC(=O)OCC(CCCCCCCC)CCCCCCCCCC] |
| III-14 | 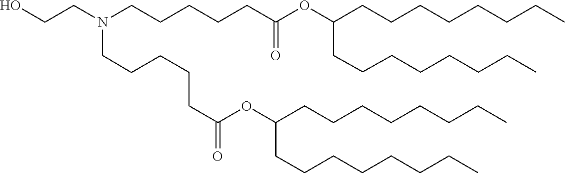 [SMILES: CCCCCCCCC(CCCCCCCC)OC(=O)CCCCCN(CCO)CCCCCC(=O)OC(CCCCCCCC)CCCCCCCC] |
| III-15 | 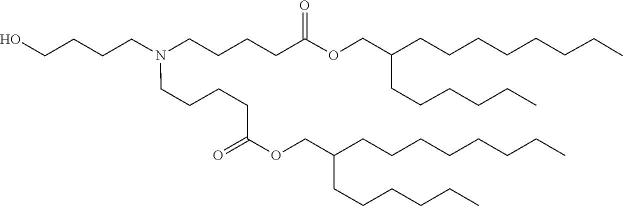 [SMILES: CCCCCCCCC(CCCCCC)COC(=O)CCCCN(CCCCO)CCCCC(=O)OCC(CCCCCC)CCCCCCCC] |
| III-16 | 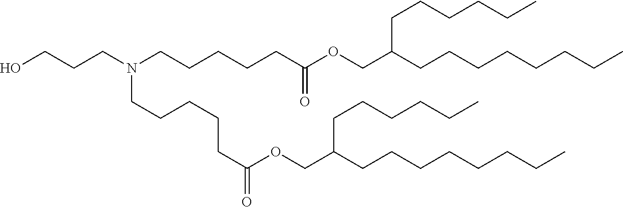 [SMILES: CCCCCCCCC(CCCCCC)COC(=O)CCCCCN(CCCO)CCCCCC(=O)OCC(CCCCCC)CCCCCCCC] |
| III-17 | 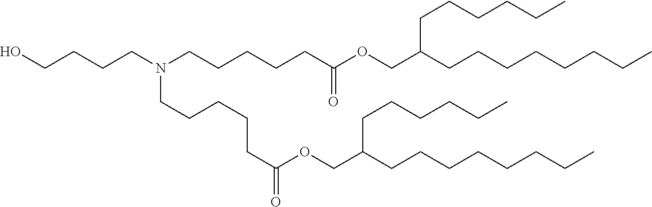 [SMILES: CCCCCCCCC(CCCCCC)COC(=O)CCCCCN(CCCCO)CCCCCC(=O)OCC(CCCCCC)CCCCCCCC] |
| III-18 | 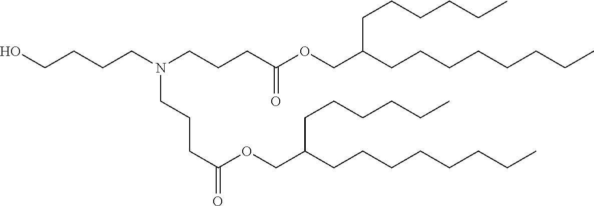 [SMILES: CCCCCCCCC(CCCCCC)COC(=O)CCCN(CCCCO)CCCC(=O)OCC(CCCCCC)CCCCCCCC] |
| III-19 | 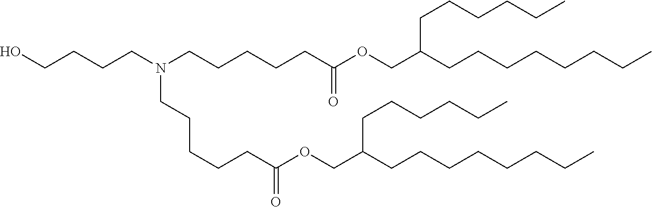 [SMILES: CCCCCCCCC(CCCCCC)COC(=O)CCCCCN(CCCCO)CCCCCC(=O)OCC(CCCCCC)CCCCCCCC] |
| III-20 | 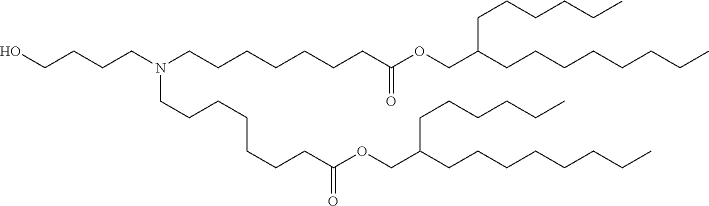 [SMILES: CCCCCCCCC(CCCCCC)COC(=O)CCCCCCCN(CCCCO)CCCCCCCC(=O)OCC(CCCCCC)CCCCCCCC] |
| III-21 | 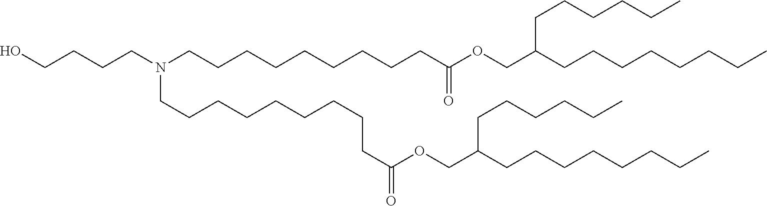 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 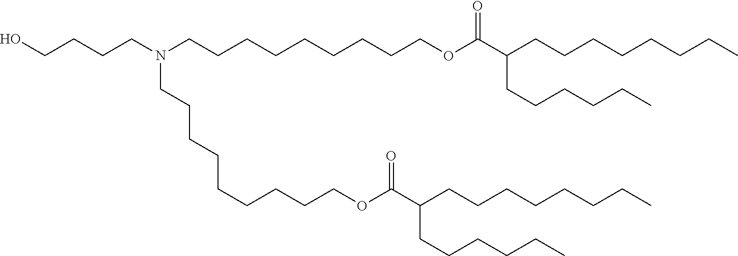 [SMILES: CCCCCCCCC(CCCCCC)C(=O)OCCCCCCCCCN(CCCCO)CCCCCCCCCOC(=O)C(CCCCCC)CCCCCCCC] |
| III-23 | 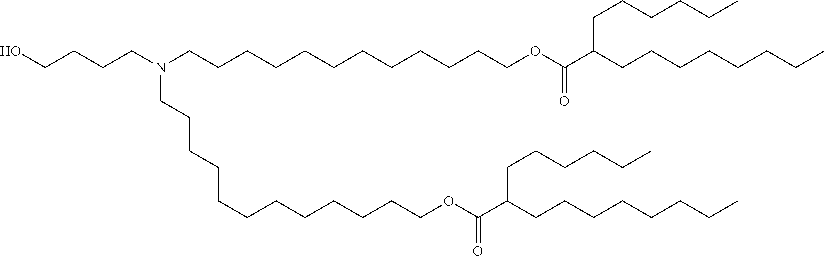 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 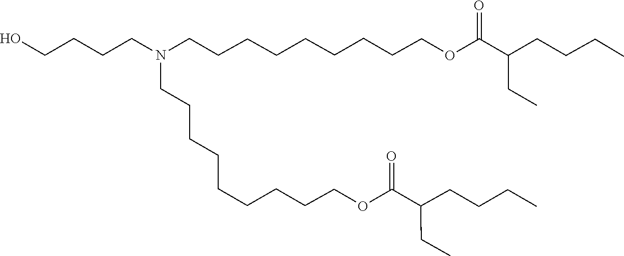 [SMILES: CCCCC(CC)C(=O)OCCCCCCCCCN(CCCCO)CCCCCCCCCOC(=O)C(CC)CCCC] |
| III-25 | 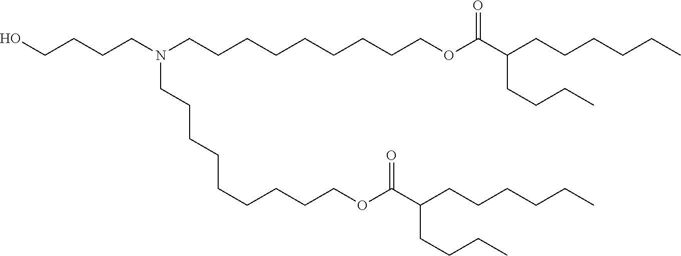 [SMILES: CCCCCCC(CCCC)C(=O)OCCCCCCCCCN(CCCCO)CCCCCCCCCOC(=O)C(CCCC)CCCCCC] |
| III-26 | 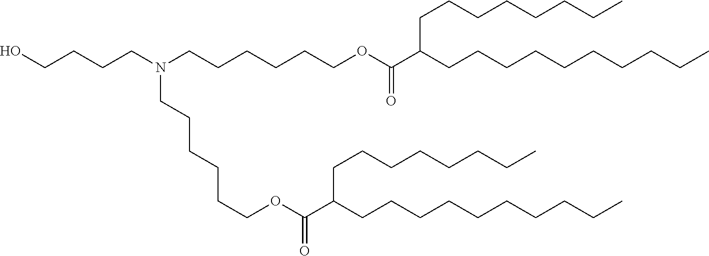 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 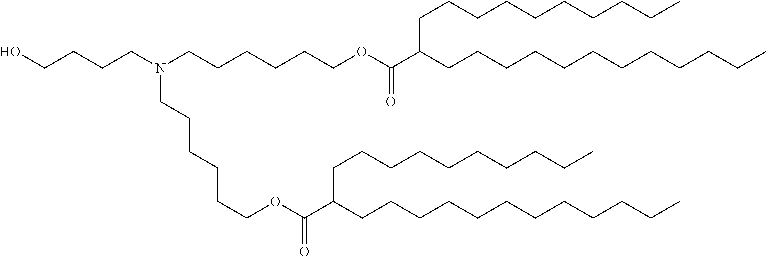 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 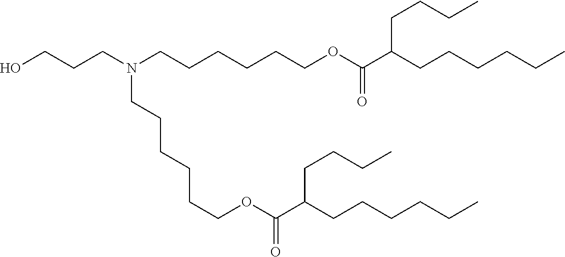 [SMILES: CCCCCCC(CCCC)C(=O)OCCCCCCN(CCCO)CCCCCCOC(=O)C(CCCC)CCCCCC] |
| III-29 | 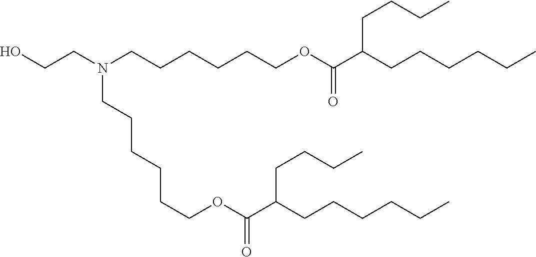 [SMILES: CCCCCCC(CCCC)C(=O)OCCCCCCN(CCO)CCCCCCOC(=O)C(CCCC)CCCCCC] |
| III-30 | 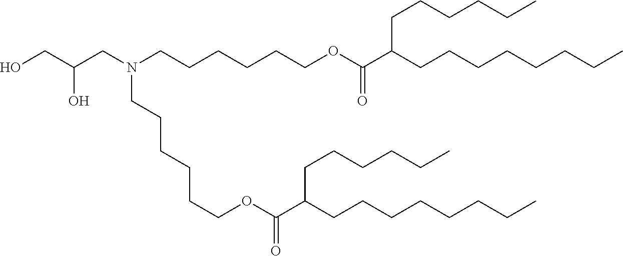 [SMILES: CCCCCCCCC(CCCCCC)C(=O)OCCCCCCN(CCCCCCOC(=O)C(CCCCCC)CCCCCCCC)CC(O)CO] |
| III-31 | 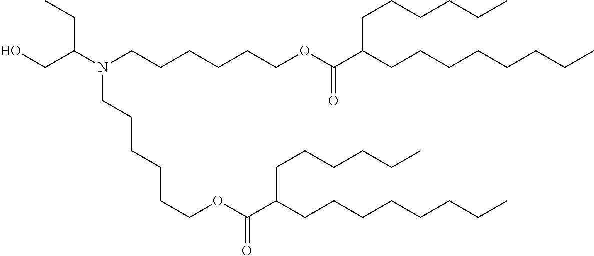 [SMILES: CCCCCCCCC(CCCCCC)C(=O)OCCCCCCN(CCCCCCOC(=O)C(CCCCCC)CCCCCCCC)C(CC)CO] |
| III-32 | 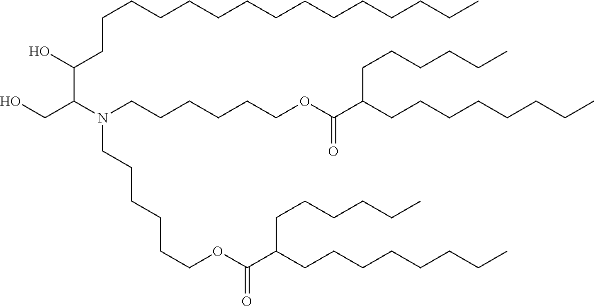 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 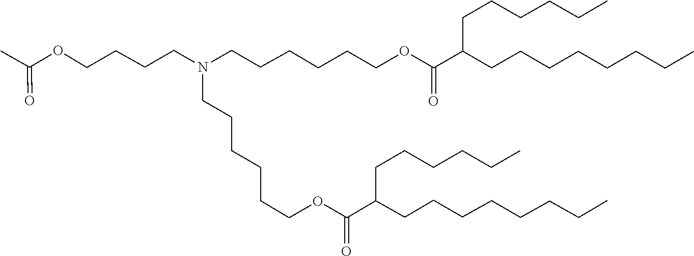 [SMILES: CCCCCCCCC(CCCCCC)C(=O)OCCCCCCN(CCCCCCOC(=O)C(CCCCCC)CCCCCCCC)CCCCOC(C)=O] |
| III-34 | 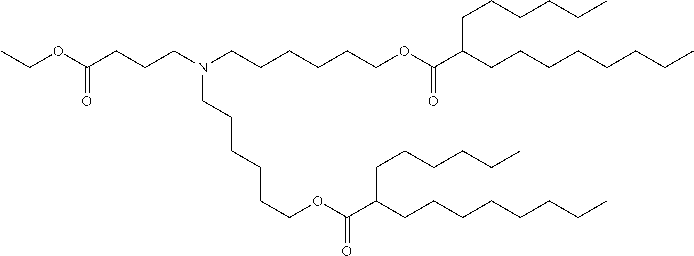 [SMILES: CCCCCCCCC(CCCCCC)C(=O)OCCCCCCN(CCCCCCOC(=O)C(CCCCCC)CCCCCCCC)CCCC(=O)OCC] |
| III-35 | 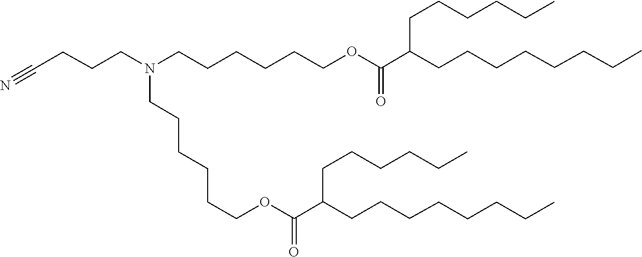 [SMILES: CCCCCCCCC(CCCCCC)C(=O)OCCCCCCN(CCCC#N)CCCCCCOC(=O)C(CCCCCC)CCCCCCCC] |
| III-36 | 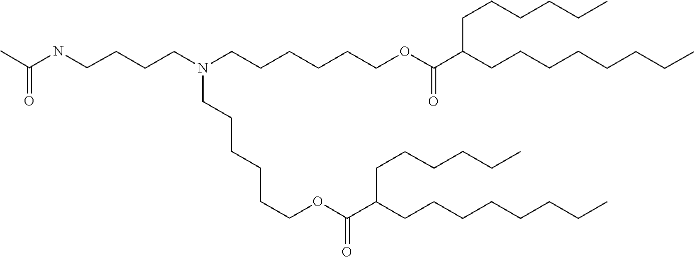 |

In some embodiments, the LNPs comprise a lipid of Formula (III), an artificial RNA of the first aspect, and one or more excipient selected from neutral lipids, steroids and PEGylated lipids. In some embodiments the lipid of Formula (III) is compound III-3. In some embodiments the lipid of Formula (III) is compound III-7.

In preferred embodiments, the LNP comprises a cationic lipid selected from:

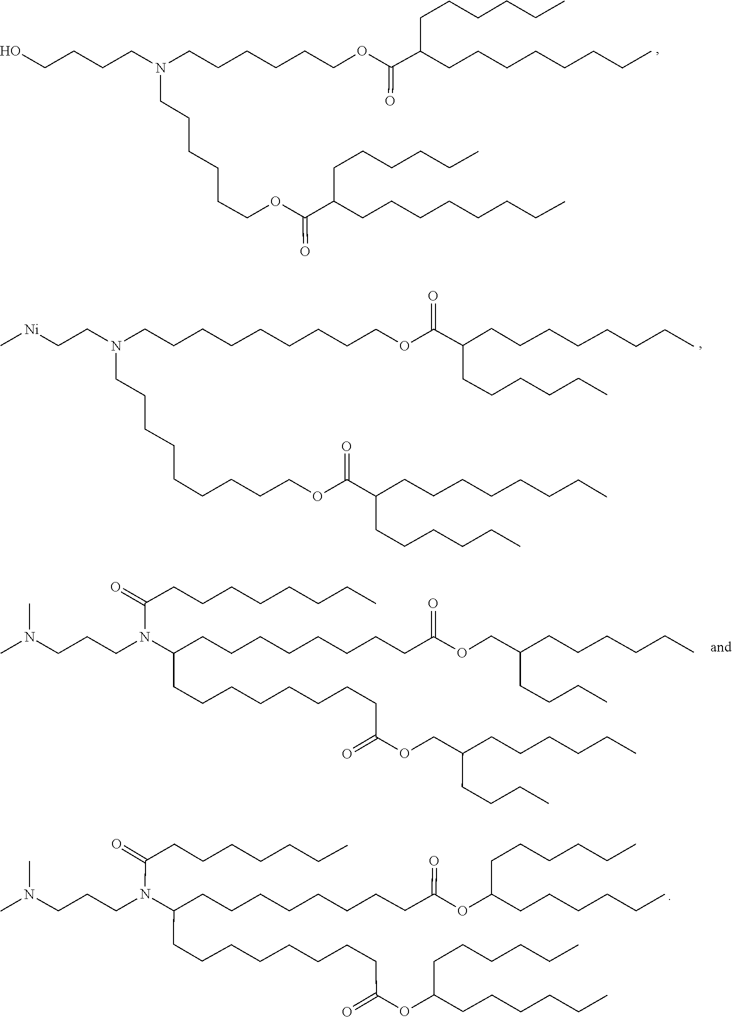

In a particularly preferred embodiment of the second aspect, the artificial RNA of the first aspect is complexed with one or more lipids thereby forming lipid nanoparticles (LNP), wherein the LNP comprises the following cationic lipid (lipid according to Formula III-3 of Table A):

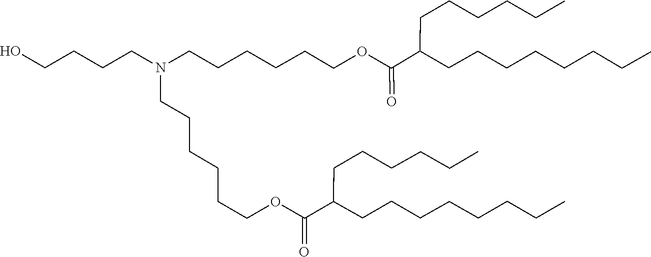 [SMILES: CCCCCCCCC(CCCCCC)C(=O)OCCCCCCN(CCCCO)CCCCCCOC(=O)C(CCCCCC)CCCCCCCC]

In certain embodiments, the cationic lipid as defined herein, preferably as disclosed in Table A, more preferably cationic lipid compound III-3, is present in the LNP in an amount from about 30 to about 95 mole percent, relative to the total lipid content of the LNP. If more than one cationic lipid is incorporated within the LNP, such percentages apply to the combined cationic lipids.

In one embodiment, the cationic lipid is present in the LNP in an amount from about 30 to about 70 mole percent. In one embodiment, the cationic lipid is present in the LNP in an amount from about 40 to about 60 mole percent, such as about 40, 41, 42, 43, 44, 45, 46, 47, 48, 49, 50, 51, 52, 53, 54, 55, 56, 57, 58, 59 or 60 mole percent, respectively. In embodiments, the cationic lipid is present in the LNP in an amount from about 47 to about 48 mole percent, such as about 47.0, 47.1, 47.2, 47.3, 47.4, 47.5, 47.6, 47.7, 47.8, 47.9, 50.0 mole percent, respectively, wherein 47.7 mole percent are particularly preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to nucleic acid, preferably to the artificial RNA of the first aspect, is from about 3 to about 15, such as from about 5 to about 13 or from about 7 to about 11.

In some embodiments of the invention the LNP comprises a combination or mixture of any the lipids described above.

Other suitable (cationic) lipids are disclosed in WO2009/086558, WO2009/127060, WO2010/048536, WO2010/054406, WO2010/088537, WO2010/129709, WO2011/153493, US2011/0256175, US2012/0128760, US2012/0027803, U.S. Pat. No. 8,158,601, WO2016/118724, WO2016/118725, WO2017/070613, WO2017/070620, WO2017/099823, and WO2017/112865. In that context, the disclosures of WO2009/086558, WO2009/127060, WO2010/048536, WO2010/054406, WO2010/088537, WO2010/129709, WO2011/153493, US2011/0256175, US2012/0128760, US2012/0027803, U.S. Pat. No. 8,158,601, WO2016/118724, WO2016/118725, WO2017/070613, WO2017/070620, WO2017/099823, and WO2017/112865 specifically relating to (cationic) lipids suitable for LNPs are incorporated herewith by reference.

In some embodiments, the lipid is selected from the group consisting of 98N12-5, C12-200, and ckk-E12.

In some embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention.

In some embodiments, the protonatable lipids have a pKa of the protonatable group in the range of about 4 to about 11, e.g., a pKa of about 5 to about 7.

LNPs can comprise two or more (different) cationic lipids. The cationic lipids may be selected to contribute different advantageous properties. For example, cationic lipids that differ in properties such as amine pKa, chemical stability, half-life in circulation, half-life in tissue, net accumulation in tissue, or toxicity can be used in the LNP. In particular, the cationic lipids can be chosen so that the properties of the mixed-LNP are more desirable than the properties of a single-LNP of individual lipids.

The amount of the permanently cationic lipid or lipidoid may be selected taking the amount of the nucleic acid cargo into account. In one embodiment, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the RNA which is used as cargo. The N/P ratio may be calculated on the basis that, for example, 1 μg RNA typically contains about 3 nmol phosphate residues, provided that the RNA exhibits a statistical distribution of bases. The “N”-value of the lipid or lipidoid may be calculated on the basis of its molecular weight and the relative content of permanently cationic and—if present—cationisable groups.

LNP in vivo characteristics and behavior can be modified by addition of a hydrophilic polymer coating, e.g. polyethylene glycol (PEG), to the LNP surface to confer steric stabilization. Furthermore, LNPs can be used for specific targeting by attaching ligands (e.g. antibodies, peptides, and carbohydrates) to its surface or to the terminal end of the attached PEG chains (e.g. via PEGylated lipids).

In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

In certain embodiments, the LNP comprises an additional, stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethypbutanedioate (PEG-S-DMG), a PEGylated ceramide (PEG-cer), or a PEG dialkoxypropylcarbamate such as w-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate.

In preferred embodiments of the second aspect, the artificial RNA of the first aspect is complexed with one or more lipids thereby forming lipid nanoparticles (LNP), wherein the LNP additionally comprises a PEGylated lipid with the Formula (IV):

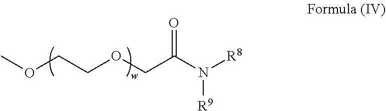

or a pharmaceutically acceptable salt, tautomer or stereoisomer thereof, wherein R⁸ and R⁹ are each independently a straight or branched, saturated or unsaturated alkyl chain containing from 10 to 30 carbon atoms, wherein the alkyl chain is optionally interrupted by one or more ester bonds; and w has mean value ranging from 30 to 60.

In some of the foregoing embodiments of the PEGylated lipid according to Formula (IV), R⁸ and R⁹ are not both n-octadecyl when w is 42. In some other embodiments, R⁸ and R⁹ are each independently a straight or branched, saturated or unsaturated alkyl chain containing from 10 to 18 carbon atoms. In some embodiments, R⁸ and R⁹ are each independently a straight or branched, saturated or unsaturated alkyl chain containing from 12 to 16 carbon atoms. In some embodiments, R⁸ and R⁹ are each independently a straight or branched, saturated or unsaturated alkyl chain containing 12 carbon atoms. In some embodiments, R⁸ and R⁹ are each independently a straight or branched, saturated or unsaturated alkyl chain containing 14 carbon atoms. In other embodiments, R⁸ and R⁹ are each independently a straight or branched, saturated or unsaturated alkyl chain containing 16 carbon atoms. In still more embodiments, R⁸ and R⁹ are each independently a straight or branched, saturated or unsaturated alkyl chain containing 18 carbon atoms. In still other embodiments, R⁸ is a straight or branched, saturated or unsaturated alkyl chain containing 12 carbon atoms and R⁹ is a straight or branched, saturated or unsaturated alkyl chain containing 14 carbon atoms.

In various embodiments, w spans a range that is selected such that the PEG portion of the PEGylated lipid according to Formula (IV) has an average molecular weight of about 400 to about 6000 g/mol. In some embodiments, the average w is about 50.

In preferred embodiments of the second aspect, R⁸ and R⁹ of the PEGylated lipid according to Formula (IV) are saturated alkyl chains.

In a particularly preferred embodiment of the second aspect, the artificial RNA of the first aspect is complexed with one or more lipids thereby forming lipid nanoparticles (LNP), wherein the LNP additionally comprises a PEGylated lipid, wherein the PEG lipid is of Formula (IVa)

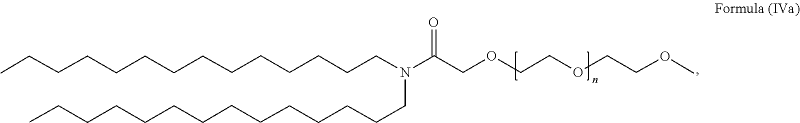

wherein n has a mean value ranging from 30 to 60, such as about 28 to about 32, about 30 to about 34, 32 to about 36, about 34 to about 38, 36 to about 40, about 38 to about 42, 40 to about 44, about 42 to about 46, 44 to about 48, about 46 to about 50, 48 to about 52, about 50 to about 54, 52 to about 56, about 54 to about 58, 56 to about 60, about 58 to about 62. In preferred embodiments, n is about 45, 46, 47, 48, 49, 50, 51, 52, 53, 54. In a most preferred embodiment n has a mean value of 49.

In other embodiments, the PEGylated lipid has one of the following structures:

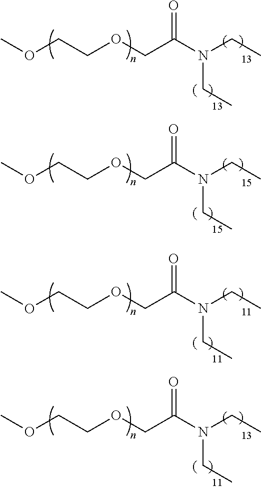

wherein n is an integer selected such that the average molecular weight of the PEGylated lipid is about 2500 g/mol, most preferably n is about 49.

Further examples of PEG-lipids suitable in that context are provided in US2015/0376115A1 and WO2015/199952, each of which is incorporated by reference in its entirety.

In some embodiments, LNPs include less than about 3, 2, or 1 mole percent of PEG or PEG-modified lipid, based on the total moles of lipid in the LNP. In further embodiments, LNPs comprise from about 0.1% to about 20% of the PEG-modified lipid on a molar basis, e.g., about 0.5 to about 10%, about 0.5 to about 5%, about 10%, about 5%, about 3.5%, about 3%, about 2.5%, about 2%, about 1.5%, about 1%, about 0.5%, or about 0.3% on a molar basis (based on 100% total moles of lipids in the LNP). In preferred embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the LNP).

In various embodiments, the molar ratio of the cationic lipid to the PEGylated lipid ranges from about 100:1 to about 25:1.

In preferred embodiments, the LNP additionally comprises one or more additional lipids which stabilize the formation of particles during their formation (e.g. neutral lipid and/or one or more steroid or steroid analogue).

In preferred embodiments of the second aspect, the artificial RNA of the first aspect is complexed with one or more lipids thereby forming lipid nanoparticles (LNP), wherein the LNP additionally comprises one or more neutral lipid and/or one or more steroid or steroid analogue.

Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

In embodiments of the second aspect, the LNP additionally comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1 carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE).

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In preferred embodiments of the second aspect, the neutral lipid is 1,2-distearoyl-sn-glycero-3-phosphocholine (DSPC). The molar ratio of the cationic lipid to DSPC may be in the range from about 2:1 to 8:1.

In preferred embodiments of the second aspect, the steroid is cholesterol. The molar ratio of the cationic lipid to cholesterol may be in the range from about 2:1 to 1:1

The sterol can be about 10 mol % to about 60 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the LNPs include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle).

Preferably, lipid nanoparticles (LNPs) comprise: (a) at least one artificial RNA of the first aspect, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In other preferred embodiments, lipid nanoparticles (LNPs) comprise: (a) at least one artificial RNA encoding prME of the first aspect and at least one artificial RNA encoding NS1 of the first aspect, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In other preferred embodiments, the composition comprises lipid nanoparticles (LNPs) comprising: (a) at least one artificial RNA encoding prME of the first aspect, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol and additionally lipid nanoparticles (LNPs) comprising (a) at least one artificial RNA encoding prME of the first aspect and at least one artificial RNA encoding NS1 of the first aspect, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In some embodiments, the LNPs comprise a lipid of Formula (III), an artificial RNA as defined above, a neutral lipid, a steroid and a PEGylated lipid. In preferred embodiments, the lipid of Formula (III) is lipid compound III-3, the neutral lipid is DSPC, the steroid is cholesterol, and the PEGylated lipid is the compound of Formula (IVa).

In a preferred embodiment of the second aspect, the LNP consists essentially of (i) at least one cationic lipid; (ii) a neutral lipid; (iii) a sterol, e.g., cholesterol; and (iv) a PEG-lipid, e.g. PEG-DMG or PEG-cDMA, in a molar ratio of about 20-60% cationic lipid:5-25% neutral lipid:25-55% sterol; 0.5-15% PEG-lipid.

In particularly preferred embodiments of the second aspect, the artificial RNA of the first aspect is complexed with one or more lipids thereby forming lipid nanoparticles (LNP), wherein the LNP essentially consists of
(i) at least one cationic lipid as defined herein, preferably a lipid of Formula MO, more preferably lipid III-3;
(ii) a neutral lipid as defined herein, preferably 1,2-distearoyl-sn-glycero-3-phosphocholine (DSPC);
(iii) a steroid or steroid analogue as defined herein, preferably cholesterol; and
(iv) a PEG-lipid as defined herein, e.g. PEG-DMG or PEG-cDMA, preferably a PEGylated lipid of Formula (IVa),
   wherein (i) to (iv) are in a molar ratio of about 20-60% cationic lipid:5-25% neutral lipid: 25-55% sterol; 0.5-15% PEG-lipid.

In one preferred embodiment, the lipid nanoparticle comprises: a cationic lipid with Formula MO and/or PEG lipid with Formula (IV), optionally a neutral lipid, preferably 1,2-distearoyl-sn-glycero-3-phosphocholine (DSPC) and optionally a steroid, preferably cholesterol, wherein the molar ratio of the cationic lipid to DSPC is optionally in the range from about 2:1 to 8:1, wherein the molar ratio of the cationic lipid to cholesterol is optionally in the range from about 2:1 to 1:1.

In a particular preferred embodiment, the composition of the second aspect comprising at least one artificial RNA of the first aspect comprises lipid nanoparticles (LNPs), which have a molar ratio of approximately 50:10:38.5:1.5, preferably 47.5:10:40.8:1.7 or more preferably 47.4:10:40.9:1.7 (i.e. proportion (mol %) of cationic lipid (preferably lipid 111-3), DSPC, cholesterol and PEG-lipid ((preferably PEG-lipid of Formula (IVa) with n=49); solubilized in ethanol).

In a particular preferred embodiment, the composition of the second aspect comprises lipid nanoparticles (LNPs), which have a molar ratio of approximately 50:10:38.5:1.5, preferably 47.5:10:40.8:1.7 or more preferably 47.4:10:40.9:1.7 (i.e. proportion (mol %) of cationic lipid (preferably lipid III-3), DSPC, cholesterol and PEG-lipid ((preferably PEG-lipid of Formula (IVa) with n=49); solubilized in ethanol), wherein the lipid nanoparticles comprise at least one RNA encoding prME, and, additionally lipid nanoparticles (LNPs), which have a molar ratio of approximately 50:10:38.5:1.5, preferably 47.5:10:40.8:1.7 or more preferably 47.4:10:40.9:1.7 (i.e. proportion (mol %) of cationic lipid (preferably lipid III-3), DSPC, cholesterol and PEG-lipid ((preferably PEG-lipid of Formula (IVa) with n=49); solubilized in ethanol), wherein the lipid nanoparticles comprise at least one RNA encoding NS1.

In a particular preferred embodiment, the composition of the second aspect comprises lipid nanoparticles (LNPs), which have a molar ratio of approximately 50:10:38.5:1.5, preferably 47.5:10:40.8:1.7 or more preferably 47.4:10:40.9:1.7 (i.e. proportion (mol %) of cationic lipid (preferably lipid III-3), DSPC, cholesterol and PEG-lipid ((preferably PEG-lipid of Formula (IVa) with n=49); solubilized in ethanol), wherein the lipid nanoparticles comprise at least one RNA encoding prME, and at least one RNA encoding NS1.

The total amount of RNA in the lipid nanoparticles may vary and is defined depending on the e.g. RNA to total lipid w/w ratio. In one embodiment of the invention the artificial RNA to total lipid ratio is less than 0.06 w/w, preferably between 0.03 w/w and 0.04 w/w.

In various embodiments, the LNP as defined herein have a mean diameter of from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm and are substantially non-toxic. As used herein, the mean diameter may be represented by the z-average as determined by dynamic light scattering as commonly known in the art.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

According to further embodiments, the composition of the second aspect may comprise at least one adjuvant. Suitably, the adjuvant is preferably added to enhance the immunostimulatory properties of the composition.

The term “adjuvant” as used herein will be recognized and understood by the person of ordinary skill in the art, and is for example intended to refer to a pharmacological and/or immunological agent that may modify, e.g. enhance, the effect of other agents (herein: the effect of the artificial nucleic acid of the invention) or that may be suitable to support administration and delivery of the composition. The term “adjuvant” refers to a broad spectrum of substances. Typically, these substances are able to increase the immunogenicity of antigens. For example, adjuvants may be recognized by the innate immune systems and, e.g., may elicit an innate immune response (that is, a non-specific immune response). “Adjuvants” typically do not elicit an adaptive immune response. In the context of the invention, adjuvants may enhance the effect of the antigenic peptide or protein provided by the artificial nucleic acid as defined herein or the polyprotein as defined herein.

In that context, the at least one adjuvant may be selected from any adjuvant known to a skilled person and suitable for the present case, i.e. supporting the induction of an immune response in a subject, e.g. in a human subject.

Accordingly, the composition of the second aspect may comprise at least one adjuvant, wherein the at least one adjuvant may be suitably selected from any adjuvant provided in of published PCT application WO2016/203025. Adjuvants disclosed in any of the claims 2 to 17 of WO2016/203025, preferably adjuvants disclosed in claim 17 of WO2016/203025 are particularly suitable, the specific content relating thereto herewith incorporated by reference.

The composition of the second aspect may comprise, besides the components specified herein, at least one further component which may be selected from the group consisting of further antigens (e.g. in the form of a peptide or protein) or further antigen-encoding nucleic acids; a further immunotherapeutic agent; one or more auxiliary substances (cytokines, such as monokines, lymphokines, interleukins or chemokines); or any further compound, which is known to be immune stimulating due to its binding affinity (as ligands) to human Toll-like receptors; and/or an adjuvant nucleic acid, preferably an immunostimulatory RNA (isRNA), e.g. CpG-RNA etc.

### Vaccine:

In a third aspect, the present invention provides a vaccine wherein the vaccine comprises at least one artificial RNA of the first aspect, or the composition of the second aspect.

Notably, embodiments relating to the composition of the second aspect may likewise be read on and be understood as suitable embodiments of the vaccine of the third aspect. Also, embodiments relating to the vaccine of the third aspect may likewise be read on and be understood as suitable embodiments of the composition of the second aspect.

The term “vaccine” will be recognized and understood by the person of ordinary skill in the art, and is for example intended to be a prophylactic or therapeutic material providing at least one epitope or antigen, preferably an immunogen. In the context of the invention the antigen or antigenic function is provided by the inventive artificial

RNA of the first aspect (said artificial RNA comprising a coding sequence encoding a antigenic peptide or protein derived from YFV prME and/or NS1) or the composition of the second aspect (comprising said artificial RNA).

In a particularly preferred embodiment of the third aspect, the vaccine comprises the artificial RNA of the first aspect, or the composition of the second aspect, wherein said artificial RNA or said composition elicits an adaptive immune response.

According to a preferred embodiment of the third aspect, the vaccine may further comprise a pharmaceutically acceptable carrier and optionally at least one adjuvant as specified in the context of the second aspect.

Suitable adjuvants in that context may be selected from adjuvants disclosed in claim 17 of WO2016/203025.

In embodiments the vaccine comprises a plurality or at least more than one of the artificial RNAs as defined in the context of the first aspect (prME and/or NS1) or the second aspect of the invention.

Any combination of mono-, bi- or multicistronic artificial nucleic acid, preferably RNA encoding the at least one antigenic peptide or protein derived from YFV, preferably derived from YFV prME, or any combination of antigens as defined herein (and optionally further antigens), provided as separate entities (containing one RNA species) or as combined entity (containing more than one RNA species, e.g. YFV prME in combination with e.g. YFV NS1 and/or YFV NS2), is understood as a vaccine according to the present invention.

The vaccine of the third aspect typically comprises a safe and effective amount of the artificial RNA of the first aspect. As used herein, “safe and effective amount” means an amount of the artificial nucleic acid, preferably the RNA, that is sufficient to significantly induce a positive modification of a disease or disorder related to an infection with a YFV. At the same time, a “safe and effective amount” is small enough to avoid serious side-effects. In relation to the vaccine or composition of the present invention, the expression “safe and effective amount” preferably means an amount of the artificial RNA that is suitable for stimulating the adaptive immune system in such a manner that no excessive or damaging immune reactions are achieved but, preferably, also no such immune reactions below a measurable level.

A “safe and effective amount” of the artificial RNA of the composition or vaccine as defined above will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying medical doctor. Moreover, the “safe and effective amount” of the artificial RNA, the composition, the vaccine may depend from application route (intradermal, intramuscular), application device (jet injection, needle injection, microneedle patch) and/or complexation (protamine complexation or LNP encapsulation). Accordingly, the suitable “safe and effective amount” has to be adapted accordingly and will be chosen and defined by the skilled person.

The vaccine or composition according to the invention can be used according to the invention for human medical purposes and also for veterinary medical purposes (mammals, vertebrates, avian species), as a pharmaceutical composition, or as a vaccine.

In a preferred embodiment, the RNA, the composition, or the vaccine according to the invention is provided in lyophilized form (as defined herein using e.g. lyophilisation methods as described in WO2016/165831, WO2011/069586, WO2016/184575 or WO2016/184576). Preferably, the lyophilized RNA, or the lyophilized composition, or the lyophilized vaccine is reconstituted in a suitable buffer, advantageously based on an aqueous carrier, prior to administration, e.g. Ringer-Lactate solution or a phosphate buffer solution.

Accordingly, the pharmaceutically acceptable carrier as used herein preferably includes the liquid or non-liquid basis of the inventive vaccine. If the inventive vaccine is provided in liquid form, the carrier will be water, typically pyrogen-free water; isotonic saline or buffered (aqueous) solutions, e.g. phosphate, citrate etc. buffered solutions. Preferably, Ringer-Lactate solution is used as a liquid basis for the vaccine or the composition according to the invention as described in WO2006/122828, the disclosure relating to suitable buffered solutions incorporated herewith by reference.

The choice of a pharmaceutically acceptable carrier as defined herein is determined, in principle, by the manner, in which the pharmaceutical composition or vaccine according to the invention is administered. The composition or vaccine can be administered, for example, systemically or locally. Routes for systemic administration in general include, for example, transdermal, oral, parenteral routes, including subcutaneous, intravenous, intramuscular, intra-arterial, intradermal and intraperitoneal injections and/or intranasal administration routes. Routes for local administration in general include, for example, topical administration routes but also intradermal, transdermal, subcutaneous, or intramuscular injections or intralesional, intracranial, intrapulmonal, intracardial, intraarticular and sublingual injections. More preferably, composition or vaccines according to the present invention may be administered by an intradermal, subcutaneous, or intramuscular route, preferably by injection, which may be needle-free and/or needle injection. Compositions/vaccines are therefore preferably formulated in liquid or solid form. The suitable amount of the vaccine or composition according to the invention to be administered can be determined by routine experiments, e.g. by using animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those which are suitable for use in lotions, creams, gels and the like. If the inventive composition or vaccine is to be administered perorally, tablets, capsules and the like are the preferred unit dose form. The pharmaceutically acceptable carriers for the preparation of unit dose forms which can be used for oral administration are well known in the prior art. The choice thereof will depend on secondary considerations such as taste, costs and storability, which are not critical for the purposes of the present invention, and can be made without difficulty by a person skilled in the art.

The inventive vaccine or composition can additionally contain one or more auxiliary substances as defined above in order to further increase the immunogenicity. A synergistic action of the nucleic acid contained in the inventive composition and of an auxiliary substance, which may be optionally be co-formulated (or separately formulated) with the inventive vaccine or composition as described above, is preferably achieved thereby. Preferably, such immunogenicity increasing agents or compounds are provided separately (not co-formulated with the inventive vaccine or composition) and administered individually.

Further additives which may be included in the inventive vaccine or composition are emulsifiers, such as, for example, Tween; wetting agents, such as, for example, sodium lauryl sulfate; colouring agents; taste-imparting agents, pharmaceutical carriers; tablet-forming agents; stabilizers; antioxidants; preservatives.

### Kit or Kit of Parts, Application, Medical Uses, Method of Treatment:

In a forth aspect, the present invention provides a kit or kit of parts, wherein the kit or kit of parts comprises the artificial RNA of the first aspect, the composition of the second aspect, or the vaccine of the third aspect, optionally comprising a liquid vehicle for solubilising, and optionally technical instructions providing information on administration and dosage of the components.

The technical instructions may contain information about administration and dosage and patient groups. Such kits, preferably kits of parts, may be applied e.g. for any of the applications or uses mentioned herein, preferably for the use of the artificial RNA, the composition comprising the artificial RNA, or the vaccine, for the treatment or prophylaxis of an infection or diseases caused by YFV or disorders related thereto. Preferably, the artificial RNA, the composition comprising the artificial RNA, or the vaccine is provided in a separate part of the kit, wherein the artificial RNA, the composition comprising the artificial RNA, or the vaccine is preferably lyophilised. The kit may further contain as a part a vehicle (e.g. buffer solution) for solubilising the artificial RNA, the composition comprising the artificial RNA, or the vaccine.

In an embodiment, where the composition of the second aspect, or the vaccine of the third aspect comprises more than on RNA species, said more than one mRNA species may be provided as separate entities (each containing one RNA species, preferably an LNP formulated RNA species, e.g. LNP formulated YFV prME mRNA and LNP formulated YFV NS1 and/or YFV NS2 mRNA) is understood as a vaccine according to the present invention.

In preferred embodiments, the kit or kit of parts as defined herein comprises Ringer lactate solution.

Any of the above kits may be used in a treatment or prophylaxis as defined herein. More preferably, any of the above kits may be used as a vaccine, preferably a vaccine against infections caused by YFV as defined herein.

### Medical Use:

In a further aspect, the present invention relates to the first medical use of the artificial RNA of the first aspect, the composition of the second aspect, the vaccine of the third aspect, or the kit or kit of parts of the forth aspect.

Accordingly, the artificial RNA of the first aspect, the composition of the second aspect, the vaccine of the third aspect, or the kit or kit of parts of the forth aspect is for use as a medicament.

The present invention furthermore provides several applications and uses of the artificial RNA of the first aspect, the composition of the second aspect, the vaccine of the third aspect, or the kit or kit of parts of the forth aspect. In particular, said RNA, composition, vaccine, or the kit or kit of parts may be used for human medical purposes and also for veterinary medical purposes, preferably for human medical purposes.

In particular, said RNA, composition, vaccine is for use as a medicament for human medical purposes, wherein said RNA, composition, vaccine may be particularly suitable for young infants, immunocompromised recipients, as well as pregnant and nursing women and elderly people.

In yet another aspect, the present invention relates to the second medical use of the artificial RNA of the first aspect, the composition of the second aspect, the vaccine of the third aspect, or the kit or kit of parts of the forth aspect.

Accordingly, the artificial RNA of the first aspect, the composition of the second aspect, the vaccine of the third aspect, or the kit or kit of parts of the forth aspect is for use in the treatment or prophylaxis of an infection with Yellow fever virus, or a disorder related to such an infection.

In particular, the artificial RNA of the first aspect, the composition of the second aspect, the vaccine of the third aspect, or the kit or kit of parts of the forth aspect may be used in the treatment or prophylaxis of an infection with flavivirus, in particular with YFV, or a disorder related to such an infection for human and also for veterinary medical purposes, preferably for human medical purposes.

As used herein, “a disorder related to a YFV infection” may preferably comprise a typical symptom or a complication of a flavivirus infection, such as, preferably a YFV infection.

Particularly, the artificial RNA of the first aspect, the composition of the second aspect, the vaccine of the third aspect, or the kit or kit of parts of the forth aspect may be used in a method of prophylactic (pre-exposure prophylaxis or post-exposure prophylaxis) and/or therapeutic treatment of infections caused by YFV.

The composition or the vaccine as defined herein, in particular the composition comprising at least one artificial nucleic acid according to the invention may preferably administered locally. In particular, composition or vaccines may be administered by an intradermal, subcutaneous, intranasal, or intramuscular route. Inventive compositions or vaccines of the invention are therefore preferably formulated in liquid (or sometimes in solid) form. In embodiments, the inventive vaccine may be administered by conventional needle injection or needle-free jet injection. Preferred in that context is the artificial RNA, the composition, the vaccine is administered by intramuscular needle injection.

The term “jet injection”, as used herein, refers to a needle-free injection method, wherein a fluid containing at least one artificial RNA of the first aspect, and, optionally, further suitable excipients is forced through an orifice, thus generating an ultra-fine liquid stream of high pressure that is capable of penetrating mammalian skin and, depending on the injection settings, subcutaneous tissue or muscle tissue. In principle, the liquid stream perforates the skin, through which the liquid stream is pushed into the target tissue. Preferably, jet injection is used for intradermal, subcutaneous or intramuscular injection of the artificial RNA, the composition, the vaccine.

In embodiments, the artificial RNA as comprised in a composition or vaccine as defined herein is provided in an amount of about 100 ng to about 500 ug, in an amount of about 1 ug to about 200 ug, in an amount of about 1 ug to about 100 ug, in an amount of about 5 ug to about 100 ug, preferably in an amount of about 1 ug to about 50 ug, specifically, in an amount of about 5 ug, 10 ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug or 100 ug.

In embodiments, where the composition of the second aspect, or the vaccine of the third aspect comprises more than on RNA species, said more than one mRNA species may be administered as separate entities (each containing one RNA species, preferably an LNP formulated RNA species, e.g. LNP formulated YFV prME mRNA and LNP formulated YFV NS1 mRNA or YFV NS2 mRNA).

Depending from application route (intradermal, intramuscular, intranasal), application device (jet injection, needle injection, microneedle patch) and/or complexation (protamine complexation or LNP encapsulation) the suitable amount has to be adapted accordingly and will be chosen and defined by the skilled person.

The immunization protocol for the treatment or prophylaxis of an infection as defined herein, i.e. the immunization of a subject against a YFV, typically comprises a series of single doses or dosages of the composition or the vaccine. A single dosage, as used herein, refers to the initial/first dose, a second dose or any further doses, respectively, which are preferably administered in order to “boost” the immune reaction.

In one embodiment, the immunization protocol for the treatment or prophylaxis of an infection as defined herein, i.e. the immunization of a subject against a YFV, comprises one single doses of the composition or the vaccine.

The treatment or prophylaxis as defined above may comprise the administration of a further active pharmaceutical ingredient. In the case of the inventive vaccine or composition, which is based on the artificial RNA of the first aspect, a polypeptide may be co-administered as a further active pharmaceutical ingredient.

For example, at least one YFV protein or peptide as described herein, or a fragment or variant thereof, may be co-administered in order to induce or enhance an immune response. Further, two distinct artificial RNAs of the first aspect may be administered at different time points, preferably in a prime-boost scenario, e.g. using a composition comprising at least one YFV polypeptide as prime vaccination and a composition/vaccine comprising at least one artificial RNA of the first aspect as boost vaccination.

Suitably, the treatment or prophylaxis as defined above comprises the administration of a further active pharmaceutical ingredient, wherein the further active pharmaceutical ingredient may be an immunotherapeutic agent that can be selected from immunoglobulins, preferably IgGs, monoclonal or polyclonal antibodies, polyclonal serum or sera, etc., most preferably immunoglobulins directed against a YFV protein or peptide as defined herein. Preferably, such a further immunotherapeutic agent may be provided as a peptide/protein or may be encoded by a nucleic acid, preferably by a DNA or an RNA, more preferably an mRNA. Such an immunotherapeutic agent allows providing passive vaccination additional to active vaccination triggered by the inventive artificial nucleic acid or by the inventive polypeptide.

### Method of Treatment and Use, Diagnostic Method and Use:

In another aspect, the present invention relates to a method of treating or preventing a disorder. In preferred embodiments, the present invention relates to a method of treating or preventing a disorder, wherein the method comprises applying or administering to a subject in need thereof the artificial RNA of the first aspect, the composition of the second aspect, the vaccine of the third aspect, or the kit or kit of parts of the forth aspect.

In preferred embodiments, the disorder is an infection with YFV, or a disorder related to such an infection.

In preferred embodiments, the present invention relates to a method of treating or preventing a disorder, wherein the method comprises applying or administering to a subject in need thereof the artificial RNA of the first aspect, the composition of the second aspect, the vaccine of the third aspect, or the kit or kit of parts of the forth aspect, wherein the subject in need is a mammalian subject or an avian subject. In particularly preferred embodiments, the mammalian subject is a human subject.

In particular, such a method may preferably comprise the steps of:
a) providing the artificial RNA of the first aspect, the composition of the second aspect, the vaccine of the third aspect, or the inventive kit or kit of parts of the forth aspect;
b) applying or administering said RNA, composition, vaccine, or kit or kit of parts to a tissue or an organism;
c) optionally, administering immunoglobulin (IgGs) against a YFV.
d) optionally, administering a further substance (adjuvant, auxiliary substance, further antigen).

According to a further aspect, the present invention also provides a method for expression of at least one polypeptide comprising at least one peptide or protein derived from a YFV, or a fragment or variant thereof, wherein the method preferably comprises the following steps:

a) providing the artificial RNA of the first aspect or the composition of the second aspect; and
b) applying or administering said RNA or composition to an expression system (cells), a tissue, an organism.

The method may be applied for laboratory, for research, for diagnostic, for commercial production of peptides or proteins and/or for therapeutic purposes. The method may furthermore be carried out in the context of the treatment of a specific disease, particularly in the treatment of infectious diseases, e.g. flavivirus infections, particularly YFV infections.

Likewise, according to another aspect, the present invention also provides the use of the artificial RNA of the first aspect, the composition of the second aspect, the vaccine of the third aspect, or the inventive kit or kit of parts of the forth aspect preferably for diagnostic or therapeutic purposes, for expression of an encoded YFV antigenic peptide or protein, e.g. by applying or administering said RNA, composition, vaccine, e.g. to a cell-free expression system, a cell (e.g. an expression host cell or a somatic cell), a tissue or an organism.

The use may be applied for a (diagnostic) laboratory, for research, for diagnostics, for commercial production of peptides or proteins and/or for therapeutic purposes. The use may be carried out in vitro, in vivo or ex vivo. The use may furthermore be carried out in the context of the treatment of a specific disease, particularly in the treatment of a flavivirus infection, particularly YFV infection or a related disorder.

In a particularly preferred embodiment, the invention provides the artificial RNA of the first aspect, the composition of the second aspect, the vaccine of the third aspect, or the inventive kit or kit of parts of the forth aspect for use as a medicament, for use in treatment or prophylaxis, preferably treatment or prophylaxis of a YFV infection or a related disorder, or for use as a vaccine.

### BRIEF DESCRIPTION OF THE DRAWINGS

FIG. 1 shows that LNP-formulated mRNA encoding X-SS-prME-XX induces PRNT titers in non-human primates after i.m. injection. Further details are provided in Example 2.

FIG. 2 shows that LNP-formulated mRNA encoding X-SS-prME-XX induces long lasting PRNT titers in non-human primates after i.m. injection. Further details are provided in Example 2.

FIG. 3 shows that UTR combinations according to the invention increase the expression of YFV prME in vitro. Data shows the result of an ICW assay on HeLa cells. Values show % of the detected YFV signal. Values normalized to 100% according to the expression of the reference (UTR combination RPL32/ALB7), also indicated by dashed horizontal line. Water for injection (WFI) serves as a control. N=4. Further details are provided in Example 3.

FIG. 4 shows that UTR combinations according to the invention increase the expression of YFV prME in vitro. Data shows the result of an ICW assay on HDF cells. Values show % of the detected YFV signal. Values normalized to 100% according to the expression of the reference (UTR combination RPL32/ALB7), also indicated by dashed horizontal line. WFI serves as a control. N=6. Further details are provided in Example 3.

FIG. 5 shows that UTR combinations according to the invention increase the expression of YFV prME in vitro. Data shows the result of a dot-blot assay using supernatants of transfected HeLa cells. Values show % of the detected YFV signal. Values normalized to 100% according to the expression of the reference (UTR combination RPL32/ALB7), also indicated by dashed horizontal line. Further details are provided in Example 3.

FIG. 6 shows that UTR combinations according to the invention increase the expression of YFV prME in vivo. Data shows the result of a FACS-based immunoassay using supernatants of vaccinated mice.

FIG. 6A shows the result of serum samples of day 14 (1:100), FIG. 6B shows the result of serum samples of day 28 (1:150). Values show % of the detected YFV signal. Values normalized to 100% according to the reference UTR combination (RPL32/ALB7) expression (also indicated by dashed horizontal line). Further details are provided in Example 4.

FIG. 7 shows that UTR combinations according to the invention increase the expression of YFV prME in vivo. Data shows the result of a FACS-based immunoassay using supernatants of vaccinated mice.

FIG. 7A shows the result of serum samples of day 56 (1:200), FIG. 7B shows the result of serum samples of day 70 (1:500). Further details are provided in Example 4.

FIG. 8 shows that a dose of 1 ug mRNA encoding X-SS-prME-XX (LNP formulated) is sufficient to induce binding antibody titers. Further details are provided in Example 5.

FIG. 9 shows that LNP-formulated mRNA encoding X-SS-prME-XX induces PRNT titers in in mice after i.m. injection Further details are provided in Example 5.

FIG. 10 shows that Capt mRNA encoding YFV prME lead to an increased expression of YFV prME in vitro in comparison to m7G (Cap0) mRNA. Further details are provided in Example 6. (FIG. 10a: Western blot, FIG. 10b: relative signal intensity).

### EXAMPLES

The examples shown in the following are merely illustrative and shall describe the present invention in a further way. These examples shall not be construed to limit the present invention thereto.

### Example 1: Preparation of mRNA Constructs and Compositions for In Vitro and In Vivo Experiments

The present Example provides methods of obtaining the artificial RNA of the invention as well as methods of generating a composition or a vaccine of the invention.

1.1. Preparation of DNA and mRNA Constructs:

For the present examples, DNA sequences encoding YFV prME were prepared and used for subsequent RNA in vitro transcription reactions. Said DNA sequences were prepared by modifying the wild type encoding DNA sequences by introducing a G/C optimized or C-maximized sequence for stabilization. Sequences were introduced into a pUC19 derived vector to comprise stabilizing 3′-UTR sequences derived from an ALB7 gene, an alpha-globin gene, a PSMB3 gene, a CASP1 gene, a COX6B1 gene, or a NDUFA1 gene and 5′-UTR sequences derived from a RPL32 gene, a HSD17B4 gene, an ATP5A1 gene, a NDUFA4 gene, a NOSIP gene, a RPL31 gene, or a SLC7A3 gene, additionally comprising, a stretch of adenosines (64A or 75A), and, optionally, a histone-stem-loop (hSL) structure and/or, a stretch of 30 cytosines (C30) as listed in Table 3.

The obtained plasmid DNA constructs were transformed and propagated in bacteria using common protocols known in the art. Eventually, the plasmid DNA constructs were extracted, purified, and used for subsequent RNA in vitro transcription (see section 1.2).

Alternatively, DNA plasmids prepared according to paragraph 1 are used as DNA template for PCR-based amplification. Eventually, the generated PCR products are purified and used for subsequent RNA in vitro transcription (see section 1.3).

1.2. RNA In Vitro Transcription from Plasmid DNA Templates:

DNA plasmids prepared according to paragraph 1.1 were enzymatically linearized using EcoRI or SapI and used for DNA dependent RNA in vitro transcription using T7 RNA polymerase in the presence of a nucleotide mixture (ATP/GTP/CTP/UTP) and cap analog (m7GpppG, m7G(5′)ppp(5′)(2′OMeA)pG, or m7G(5′)ppp(5)(2′OMeG)pG) under suitable buffer conditions. The obtained mRNA constructs were purified using RP-HPLC (PureMessenger®, CureVac AG, Tubingen, Germany; WO2008/077592) and used for in vitro and in vivo experiments. RNA for clinical development (see Example 6) is produced under current good manufacturing practice e.g. according to WO2016/180430, implementing various quality control steps on DNA and RNA level. The generated RNA sequences/constructs are provided in Table 3 with the encoded YFV protein, the UTR elements, and the 3′-terminal end indicated therein. In addition to the information provided in Table 3, further information relating to specific mRNA construct SEQ-ID NOs may be derived from the information provided under <223> identifier provided in the ST.25 sequence listing.

Alternatively, EcoRI or SapI linearized DNA is used for DNA dependent RNA in vitro transcription using an RNA polymerase in the presence of a modified nucleotide mixture (ATP, GTP, CTP, N(1)-methylpseudouridine (m14P) or pseudouridine (Ψ) and cap analog (m7GpppG, m7G(5)ppp(5′)(2′OMeA)pG, or m7G(5′)ppp(5′)(2′OMeG)pG) under suitable buffer conditions. The obtained m14Ψ- or Ψ-modified mRNAs are purified using RP-HPLC (PureMessenger®, CureVac, Tubingen, Germany; WO2008/077592) and used for further experiments.

Some mRNA constructs are in vitro transcribed in the absence of a cap analog. The cap-structure (cap1) is added enzymatically using Capping enzymes as commonly known in the art. In short, in vitro transcribed mRNA is capped using an m7G capping kit with 2′-O-methyltransferase to obtain cap1-capped mRNA. Cap1-capped mRNA is purified using RP-HPLC (PureMessenger®, CureVac, Tubingen, Germany; WO2008/077592) and used for further experiments.

1.3. RNA In Vitro Transcription from PCR Amplified DNA Templates:

Purified PCR amplified DNA templates prepared according to paragraph 1.1 are transcribed in vitro using DNA dependent T7 RNA polymerase in the presence of a nucleotide mixture (ATP/GTP/CTP/UTP) and cap analog (m7GpppGunder suitable buffer conditions. Alternatively, PCR amplified DNA is transcribed in vitro using DNA dependent T7 RNA polymerase in the presence of a modified nucleotide mixture (ATP, GTP, CTP, N(1)-methylpseudouridine (m14′) or pseudourinde (Ψ)) and cap analog (m7GpppG, m7G(5′)ppp(5)(2′OMeA)pG or m7G(5′)ppp(5′)(2′OMeG)pG) under suitable buffer conditions. Some mRNA constructs are in vitro transcribed in the absence of a cap analog and the cap-structure (cap1) is added enzymatically using capping enzymes as commonly known in the art e.g. using an m7G capping kit with 2′-O-methyltransferase. The obtained mRNAs are purified e.g. using RP-HPLC (PureMessenger®, CureVac AG, Tubingen, Germany; WO2008/077592) and used for in vitro and in vivo experiments.

TABLE 3
mRNA constructs used in the present examples (Example 1)

| | | 5′-UTR/3′-URT; | | SEQ |
|---|---|---|---|---|
| RNA ID | construct | UTR Design | 3′-end | ID NO: |
| R2388/ | X-SS-prME-XX (opt1) | 3′-UTR of an alpha- | A64-N5-C30-hSL-N5 | 1194 |
| R2571/ | | globin gene (muag); i-3 | | |
| R6711 | | | | |
| R2581/ | X-SS-prME-XX (opt1) | RPL32/ALB7; i-2 | A64-N5-C30-hSL-N5 | 1074 |
| R2582/ | | | | |
| R3911 | | | | |
| R5351 | X-SS-prME-XX (opt1) | HSD17B4/PSMB3; a-1 | A64-N5 | 1308 |
| R5353 | X-SS-prME-XX (opt1) | HSD17B4/CASP1; b-4 | A64-N5 | 1310 |
| R5356 | X-SS-prME-XX (opt1) | SLC7A3/NDUFA1; d-5 | A64-N5 | 1313 |
| R5357 | X-SS-prME-XX (opt1) | ATP5A1/PSMB3; c-5 | A64-N5 | 1311 |
| R5360 | X-SS-prME-XX (opt1) | N0SIP/CASP1; g-4 | A64-N5 | 1314 |
| R5362 | X-SS-prME-XX (opt1) | RPL31/PSMB3; d-1 | A64-N5 | 1312 |
| R5364 | X-SS-prME-XX (opt1) | N0SIP/PSMB3; a-4 | A64-N5 | 1320 |
| R5367 | X-SS-prME-XX (opt1) | SLC7A3/CASP1; h-4 | A64-N5 | 1315 |
| R5368 | X-SS-prME-XX (opt1) | SLC7A3/COX6B1; h-5 | A64-N5 | 1316 |
| R5369 | X-SS-prME-XX (opt1) | NDUFA4/PSMB3; a-2 | A64-N5 | 1309 |
| R5372 | X-SS-prME-XX (opt1) | RPL32/ALB7; i-2 | A64-N5 | 1318 |
| R7229 | X-SS-prME-XX (opt1) | NDUFA4/PSMB3; a-2 | A64-N5-C30-hSL-N5 | 594 |
| R7230 | X-SS-prME-XX (opt1) | N0SIP/PSMB3; a-4 | A64-N5-C30-hSL-N5 | 1254 |
| R7231 | X-SS-prME-XX (opt1) | ATP5A1/PSMB3; c-5 | A64-N5-C30-hSL-N5 | 714 |
| R7232 | X-SS-prME-XX (opt1) | HSD17B4/CASP1; b-4 | A64-N5-C30-hSL-N5 | 654 |
| R7233 | X-SS-prME-XX (opt1) | HSD17B4/PSMB3; a-1 | A64-N5-C30-hSL-N5 | 534 |
| R8481/ | X-SS-prME-XX (opt1) | 3′-UTR of an alpha- | hSL-A64-N5 | 2171 |
| R8482 | | globin gene (muag); i-3 | | |
| R8483 | X-SS-prME-XX (opt1) | HSD17B4/PSMB3; a-1 | hSL-A100 | 2190 |
| R8484 | X-SS-prME-XX (opt1) | 3′-UTR of an alpha- | A64-N5-hSL-N5 | 2183 |
| | | globin gene (muag); i-3 | | |
| R7253 | X-SS-prME-XX (opt11) | 3′-UTR of an alpha- | A64-N5-C30-hSL-N5 | 1242 |
| | | globin gene (muag); i-3 | | |
| R7250 | X-SS-prME-XX (opt2) | 3′-UTR of an alpha- | A64-N5-C30-hSL-N5 | 1212 |
| | | globin gene (muag); i-3 | | |
| R7251 | X-SS-prME-XX (opt4) | 3′-UTR of an alpha- | A64-N5-C30-hSL-N5 | 1224 |
| | | globin gene (muag); i-3 | | |
| R7252 | X-SS-prME-XX (opt6) | 3′-UTR of an alpha- | A64-N5-C30-hSL-N5 | 1236 |
| | | globin gene (muag); i-3 | | |
| R2387 | X-SS-prME-XX (wt) | 3′-UTR of an alpha- | A64-N5-C30-hSL-N5 | 1188 |
| | | globin gene (muag); i-3 | | |
| R8491/ | X-SS-prME (opt1) | 3′-UTR of an alpha- | hSL-A64-N5 | 2173 |
| R8492 | | globin gene (muag); i-3 | | |
| R8493 | X-SS-prME (opt1) | HSD17B4/PSMB3; a-1 | hSL-A100 | 2191 |
| R2605/ | SS-prME (opt1) | 3′-UTR of an alpha- | A64-N5-C30-hSL-N5 | 1195 |
| R2606 | | globin gene (muag); i-3 | | |
| R2607/ | SS-prME (opt1) | RPL32/ALB7; i-2 | A64-N5-C30-hSL-N5 | 1075 |
| R2608 | | | | |
| R8488/ | SSjev(V3)-prME-XX (opt1) | 3′-UTR of an alpha- | hSL-A64-N5 | 2174 |
| R8489 | | globin gene (muag); i-3 | | |
| R8490 | SSjev(V3)-prME-XX (opt1) | HSD17B4/PSMB3; a-1 | hSL-A100 | 2192 |
| R8494/ | X-SS-prMEdelstem_TM-JEV (opt1) | 3′-UTR of an alpha- | hSL-A64-N5 | 2175 |
| R8495 | | globin gene (muag); i-3 | | |
| R8496 | X-SS-prMEdelstem_TM-JEV (opt1) | HSD17B4/PSMB3; a-1 | hSL-A100 | 2193 |
| R8497/ | SSjev(V3)-prMEdelstem_TM-JEV | 3′-UTR of an alpha- | hSL-A64-N5 | 2176 |
| R8498 | (opt1) | globin gene (muag); i-3 | | |
| R8499 | SSjev(V3)-prMEdelstem_TM-JEV | HSD17B4/PSMB3; a-1 | hSL-A100 | 2194 |
| | (opt1) | | | |
| R8501/ | eSS-NS1-Y (opt1) | 3′-UTR of an alpha- | hSL-A64-N5 | 2579 |
| R8502 | | globin gene (muag); i-3 | | |
| R8503 | eSS-NS1-Y (opt1) | HSD17B4/PSMB3; a-1 | hSL-A100 | 2588 |
| R8504/ | eSS-NS1 (opt1) | 3′-UTR of an alpha- | hSL-A64-N5 | 2580 |
| R8505 | | globin gene (muag); i-3 | | |
| R8506 | eSS-NS1 (opt1) | HSD17B4/PSMB3; a-1 | hSL-A100 | 2589 |
| R8507/ | SSIgE-NS1 (opt1) | 3′-UTR of an alpha- | hSL-A64-N5 | 2581 |
| R8508 | | globin gene (muag); i-3 | | |
| R8509 | SSIgE-NS1 (opt1) | HSD17B4/PSMB3; a-1 | hSL-A100 | 2590 |

1.4. Preparation of an LNP Formulated mRNA Composition:

Lipid nanoparticles (LNP), cationic lipids, and polymer conjugated lipids (PEG-lipid) were prepared and tested essentially according to the general procedures described in WO2015/199952, WO2017/004143 and WO2017/075531, the full disclosures of which are incorporated herein by reference. LNP formulated mRNA was prepared using an ionizable amino lipid (cationic lipid), phospholipid, cholesterol and a PEGylated lipid. Briefly, cationic lipid compound of Formula III-3, DSPC, cholesterol, and PEG-lipid of Formula IVa were solubilized in ethanol at a molar ratio (%) of approximately 50:10:38.5:1.5 or 47.5:10:40.9:1.7. LNPs comprising cationic lipid compound III-3 and PEG-lipid compound IVa were prepared at a ratio of mRNA to total Lipid of 0.03-0.04 w/w. The mRNA was diluted to 0.05 mg/mL to 0.2 mg/mL in 10 mM to 50 mM citrate buffer, pH 4. Syringe pumps were used to mix the ethanolic lipid solution with the mRNA aqueous solution at a ratio of about 1:5 to 1:3 (vol/vol) with total flow rates above 15m1/min. The ethanol was then removed and the external buffer replaced with a PBS buffer comprising Sucrose by dialysis. Finally, the lipid nanoparticles were filtered through a 0.2 um pore sterile filter and the LNP-formulated mRNA composition was adjusted to about 1 mg/ml total mRNA. Lipid nanoparticle particle diameter size was 60-90 nm as determined by quasi-elastic light scattering using a Malvern Zetasizer Nano (Malvern, UK). For other cationic lipid compounds mentioned in the present specification, the formulation process is essentially similar. The obtained LNP-formulated mRNA composition (1 mg/ml total mRNA) was diluted to the desired target concentration using Saline before in vivo application.

1.5. Preparation of a Protamine Complexed mRNA Composition:

mRNA constructs are complexed with protamine prior to use in in vivo immunization experiments. The mRNA formulation consisted of a mixture of 50% free mRNA and 50% mRNA complexed with protamine at a weight ratio of 2:1. First, mRNA was complexed with protamine by addition of protamine-Ringer's lactate solution to mRNA. After incubation for 10 minutes, when the complexes were stably generated, free mRNA is added, and the final concentration is adjusted with Ringer's lactate solution.

1.6. Preparation of Polymer-Lipidoid Complexed mRNA Composition:

mRNA constructs are complexed with a polymer-lipidoid prior to use in in vivo immunization experiments. 20 mg peptide (CHHHHHHRRRRHHHHHHC-NH2; SEQ ID NO: 55) TFA salt are dissolved in 2 mL borate buffer pH 8.5 and stirred at room temperature for approximately 18h. Then, 12.6 mg PEG-SH 5000 (Sunbright) dissolved in N-methylpyrrolidone is added to the peptide solution and filled up to 3 mL with borate buffer pH 8.5. After 18h incubation at room temperature, the reaction mixture is purified and concentrated by centricon procedure (MWCO 10 kDa), washed against water, and lyophilized. The obtained lyophilisate is dissolved in ELGA water and the concentration of the polymer is adjusted to 10 mg/mL. The obtained polyethylene glycol/peptide polymers (HO-PEG 5000-S-(S-CHHHHHHRRRRHHHHHHC-S-)7-S-PEG 5000-OH) are used for further formulation. Lipidoid component 3-C12 is obtained by acylation of tris(2-aminoethyl)amine with an activated lauric (C12) acid derivative, followed by reduction of the amide. Alternatively, it may be prepared by reductive amination with the corresponding aldehyde. Lipidoid 3-C12-OH is prepared by addition of the terminal C12 alkyl epoxide with the same oligoamine essentially according to e.g. Love et al., pp. 1864-1869, PNAS, vol. 107 (2010). Ringer lactate buffer (RiLa; alternatively e.g. saline (NaCl) or PBS buffer may be used), respective amounts of lipidoid, and respective amounts of said polymer are mixed to prepare compositions comprising a lipidoid and a peptide or polymer. Then, the polymer-lipidoid carrier compositions are used to assemble nanoparticles with the mRNA by mixing the mRNA with respective amounts of polymer-lipidoid carrier by incubation of 10 min at RT. In order to characterize the integrity of the obtained polymer-lipidoid complexed mRNA particles, RNA agarose gel shift assays are performed. In addition, size measurements are performed (gel shift assay, Zetasizer) to evaluate whether the obtained nanoparticles have a uniform size profile.

### Example 2: Vaccination of NHP with LNP Formulated YFV mRNA Vaccine

The present Example shows that LNP-formulated mRNA encoding YFV prME according to the invention induces long lasting PRNT titers in vaccinated NHPs.

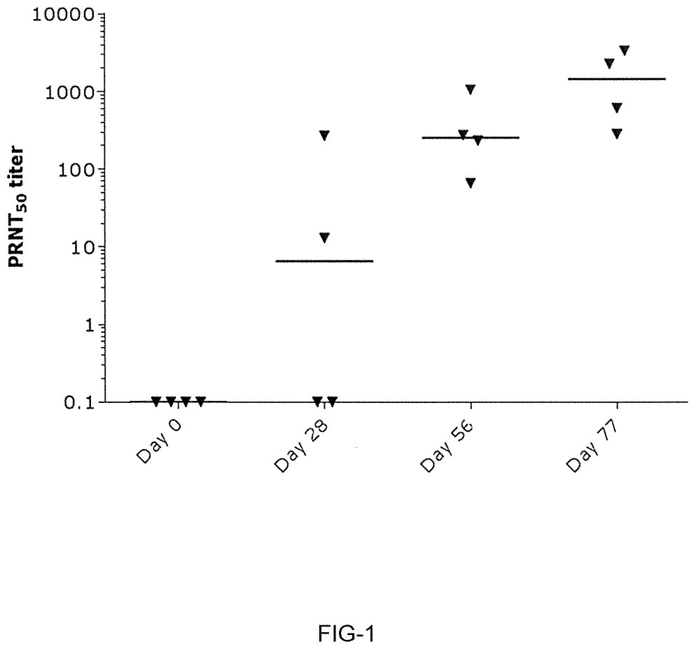

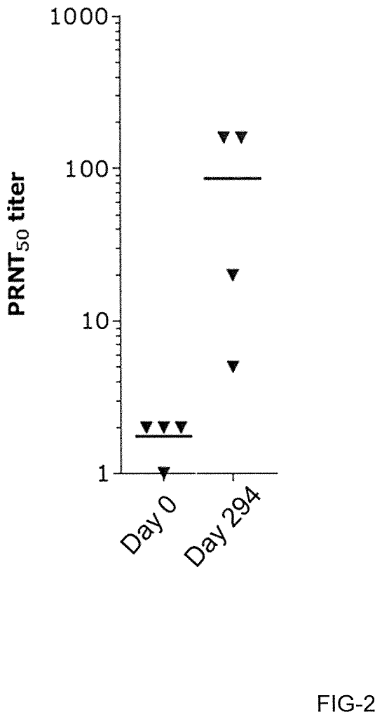

Cynomolgus monkeys (Macaca fascicularis, two male and two female) were vaccinated intramuscularly on days 0, 28 and 56 with 10 ug LNP-formulated YFV X-SS-prME-XX mRNA (R3911). Animals received a single intramuscular injection in biceps femoris muscle at the day of vaccination. Serum samples were collected from all animals at day 0, 28, 56, and 77 to determine immunogenicity. Immune response to yellow fever virus was assessed in a plaque reduction neutralizing titer (PRNT) using an attenuated YFV 17D strain. The results of the PRNT assay are shown in FIG. 1. For assessing the durability of the immune responses, serum samples collected from all animals at day 294 were analyzed (see FIG. 2).

### 2.1. Plaque Reduction Neutralization Test (PRNT50):

Sera are analyzed by a plaque reduction neutralization test (PRNT50), performed as commonly known in the art. Briefly, obtained serum samples of vaccinated NHPs were incubated with YFV. That mixture is used to infect cultured cells, and the reduction in the number of plaques was determined. The results of the PRNT assay are shown in FIGS. 1 and 2.

### 2.2. Results:

As shown in FIG. 1, immunization of Cynomolgus macaques with LNP-formulated mRNA based vaccine led to the production of YF-specific neutralizing antibodies. The results of FIG. 2 further demonstrate that induced immune responses have a remarkable longevity as all vaccinated animals maintained high PRNT titers at day 294 post prime vaccination.

To further improve the efficiency of the mRNA-based vaccine, several alternative YFV prME mRNA constructs were designed harboring different UTR combinations to potentially increase translation efficiency of the mRNA. Those mRNA constructs were tested in vitro (see Example 3) and in vivo (see e.g. Example 4).

### Example 3: In Vitro Expression Screen of YFV mRNA Constructs in Cell Western (ICW) and Dot-Blot Analysis

The present Example shows that the UTR combinations according to the invention strongly improve the expression performance of said mRNA constructs compared to a reference mRNA construct (harboring RPL32/ALB7 UTRs) used e.g. in Example 2.

To determine in vitro protein expression performance of YFV prME mRNA constructs comprising different UTR combinations (mRNA constructs used: R5351, R5369, R5353, R5357, R5362, R5356, R5360, R5367, R5368, R5372, R5364 see Example 1, Table 3), different cell types were transiently transfected with said mRNA constructs and YFV prME antigen expression was analyzed using in cell western analysis (for HeLa, and HDF cells) and dot-blot analysis (for HeLa cells).

### 3.1. In Cell Western (ICW) Analysis on HeLa and HDF Cells:

HeLa cells and HDF cells were analyzed via ICW according to the following protocol: Cells were seeded on 96 well plates with black rim and clear optical bottom (Nunc Microplate; Thermo Fisher). HeLa cells or HDF (10,000 cells in 200 ul/well) were seeded 24h before transfection in a compatible complete cell medium. Cells were maintained at 37° C., 5% CO2. The day of transfection, the complete medium on HeLa or HDF was replaced with serum-free Opti-MEM medium (Thermo Fisher). Lipocomplexed mRNAs (Lipofectamine) were added to cells for transfection with 200 ng of RNA (HeLa & HDF) per well in a total volume of 150 ul. 90 min post start of transfection, 100 ul/well of transfection solution on HeLa or HDF was exchanged for 100 ul/well of complete medium. Cells were further maintained at 37° C., 5% CO2 before performing ICW.

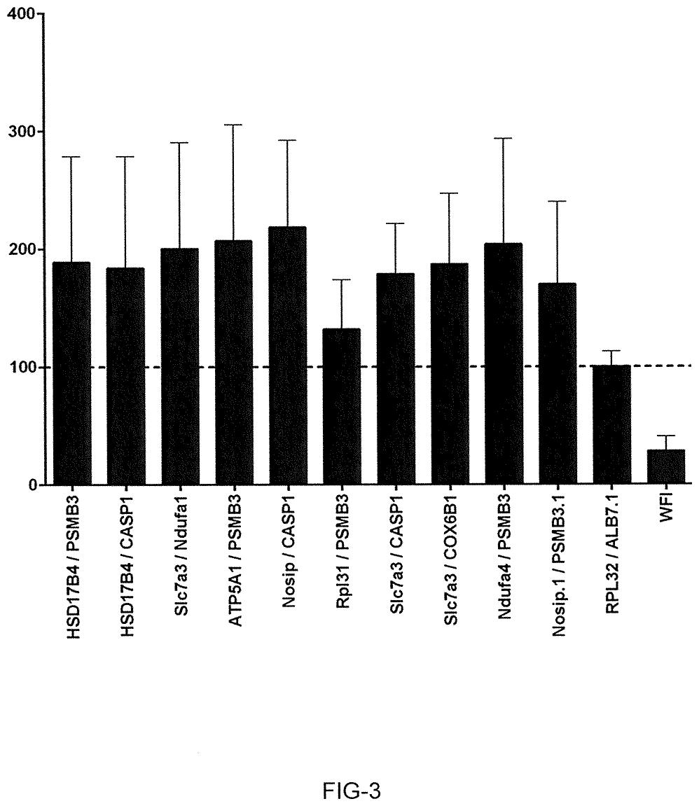

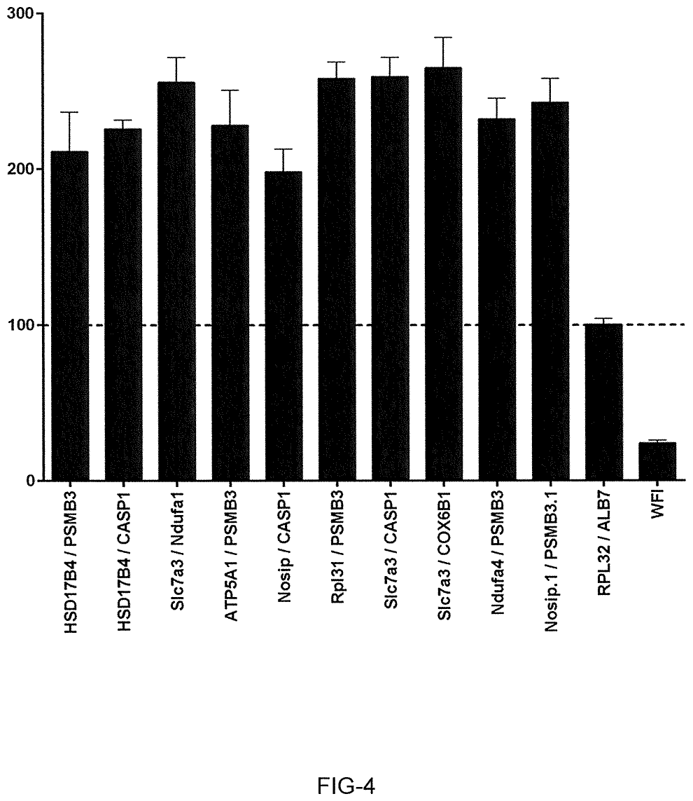

36h post start of transfection, YFV prME expression was quantified by ICW according to the following procedure (all steps performed at room temperature): First, cells were washed once with PBS and fixed with 3.7% formaldehyde in PBS for 10 min. After washing once in PBS, cells were permeabilized with Perm/Wash buffer (BD) for 30 min. Cells were blocked for 30 min with a mix of Odyssey blocking buffer (PBS) (LI-COR) and Perm/Wash buffer (BD) (1:1). Next, cells were incubated for 150 min with primary antibody directed against YFV prME (mouse monoclonal anti-YF (3576); Santa Cruz SC-58083/F1714; diluted 1:200 in BD). Cells were then washed 3 times (Perm/Wash buffer (BD)). Subsequently, cells were incubated with a mixture of secondary antibody (IRDye-coupled secondary antibody (IRDye 800CW goat anti-rabbit IgG; LI-COR; diluted 1:200 in BD) and Cell-Tag 700 Stain (LI-COR) (1:1000 in BD) for 1 h in the dark. After washing 4 times in BD, PBS was added to cells and plates scanned using an Odyssey® CLx Imaging system (LI-COR). Fluorescence (800 nm) was quantified using Image Studio Lite Software, normalized to the Cell-Tag 700 Stain and the results compared to expression from a reference construct containing the RPL32/ALB7 UTR-combination, set to a level of 100% expression. The results of the analysis are shown in FIG. 3 (HeLa cells) and FIG. 4 (HDF cells).

### 3.2. Dot Blot Analysis on HeLa Cells:

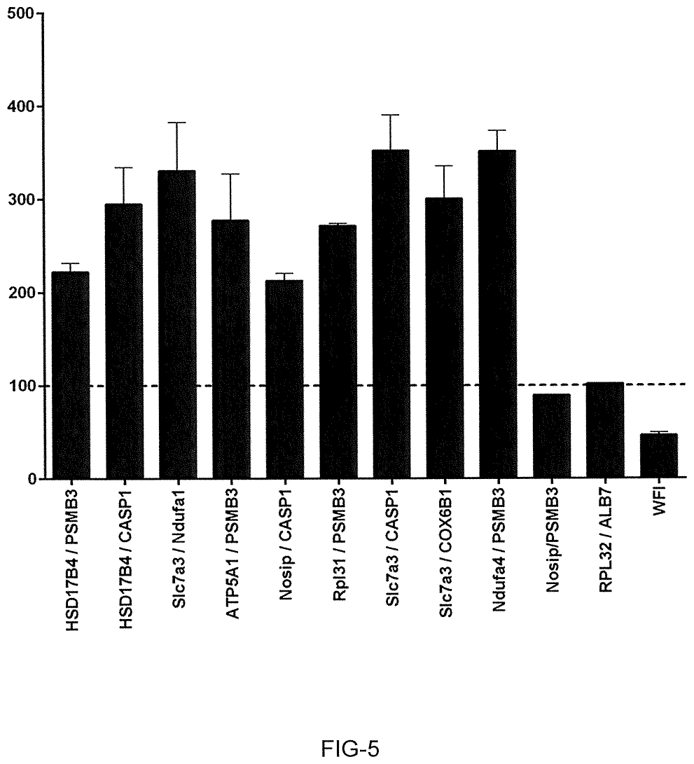

HeLa cells were seeded in a 24 well plate at a density of 300,000 cells/well in cell culture medium (RPMI, 10% FCS, 1% L-Glutamine, 1% Pen/Strep), 24h prior to transfection in a compatible complete cell medium. Cells were maintained at 37° C., 5% CO2. The day of transfection, the complete medium on HeLa was replaced with serum-free Opti-MEM medium (Thermo Fisher). Lipocomplexed mRNA (Lipofectamine) was added to cells for transfection with 500 ng of RNA per well in a total volume of 1000 ul. 90 min post start of transfection, transfection solution was exchanged of complete medium. Cells were further maintained at 37° C., 5% CO2. Supernatants were harvested 24h post transfection and 200 ul of each supernatant was used to performing Dot blot analysis. Non-specific sites of the membrane were blocked in blocking buffer (5% (w/v) skim milk powder in TBS with 0.1% Tween-20) for 1h at 4° C. on a shaker. Next, the membrane was incubated in primary antibody dilution (5 ul mouse anti-Flavivirus group antigen antibody; clone D1-4G2-4-15 (Millipore, 1:2000) in 10m1 dilution buffer (0.5% (w/v) skim milk powder in TBS with 0.1% Tween-20)) for 2h at RT in a 100m1 falcon tube on a rotating shaker. After 3×10 min washing steps in washing buffer (1×TBS with 0.1% Tween 20), the membrane was incubated in secondary antibody (goat anti-mouse IgG (H+L) IRDye 800CW; LI-COR Biosciences; 1:10000) for 1h at RT in the dark. After 3×10 min washing steps in washing buffer, the membrane was placed in TBS and subsequently imaged using an Odyssey CLx image system. The results were compared to the expression from a reference construct containing the RPL32/ALB7 UTR-combination which was set to a level of 100%. The results of the analysis are shown in FIG. 5.

### 3.3. Results:

As shown in FIGS. 3-5, the expression performances of the mRNA constructs comprising UTR combinations according to the invention were strongly increased compared to the construct comprising the reference UTR combination (RPL32/ALB7). Notably, the increase in expression was observed in different cell types (HeLa, HDF) using different in vitro assays (ICW, dot-blot). The herein identified advantageous UTR combinations were further analyzed in vivo (see Example 4).

### Example 4: Vaccination of Mice with mRNA Encoding YFV prME

The present Example shows that specific UTR combinations according to the invention also improved the expression performance of mRNA constructs in vivo. The data furthermore shows that mice vaccinated with said improved mRNA constructs show much higher humoral immune responses compared to mice vaccinated with a reference mRNA (harboring RPL32/ALB7 UTRs) used e.g. in Example 2.

### 4.1. Immunization Procedure:

Female BALB/c mice (8 animals per group) were injected intramuscularly (i.m.) with 50 ug non-formulated mRNA per dose. As a negative control, one group of mice (5 animals) was injected with buffer (ringer lactate). All animals were injected on day 0, 28 and 56. Blood samples were collected on day 14, 28, 56, and 70 for the determination of antibody titers. Further details are provided in Table 4 below.

TABLE 4
Vaccination regimen (Example 4):

| | RNA ID/SEQ | | UTR description | Volume per |
|---|---|---|---|---|
| Group | ID NO: | Construct | (5′-UTR/3′-UTR) | injection |
| 1 | R5353/1310 | X-SS-prME-XX (opt1) | HSD17B4/CASP1 | 2 × 25 ul |
| 2 | R5356/1313 | X-SS-prME-XX (opt1) | SLC7A3/NDUFA1 | 2 × 25 ul |
| 3 | R5357/1311 | X-SS-prME-XX (opt1) | ATP5A1/PSMB3 | 2 × 25 ul |
| 4 | R5360/1314 | X-SS-prME-XX (opt1) | NOSIP/CASP1 | 2 × 25 ul |
| 5 | R5362/1312 | X-SS-prME-XX (opt1) | RPL31/PSMB3 | 2 × 25 ul |
| 6 | R5367/1315 | X-SS-prME-XX (opt1) | SLC7A3/CASP1 | 2 × 25 ul |
| 7 | R5368/1316 | X-SS-prME-XX (opt1) | SLC7A3/COX6B1 | 2 × 25 ul |
| 8 | R5369/1309 | X-SS-prME-XX (opt1) | NDUFA4/PSMB3 | 2 × 25 ul |
| 9 | R5372/1318 | X-SS-prME-XX (opt1) | RPL32/ALB7 | 2 × 25 ul |
| 10 | RiLa | | | 2 × 25 ul |

### 4.2. Detection of Antigen Specific Humoral Immune Responses:

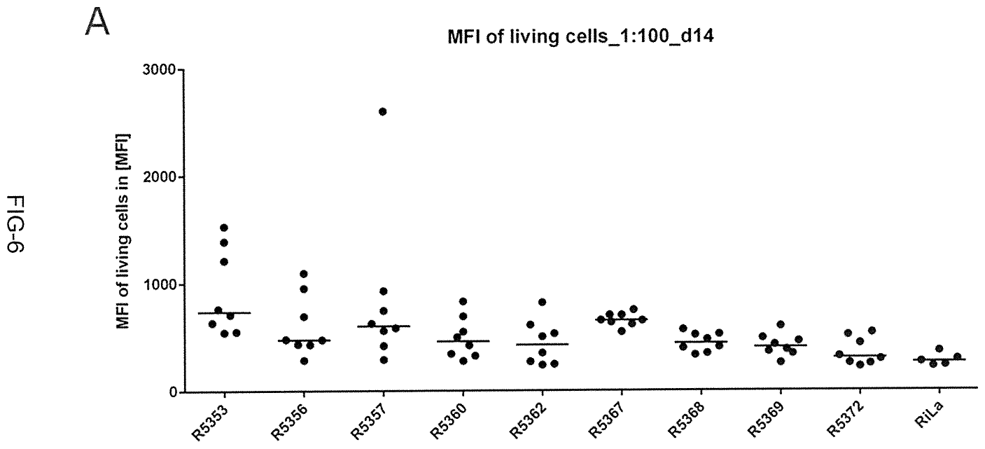

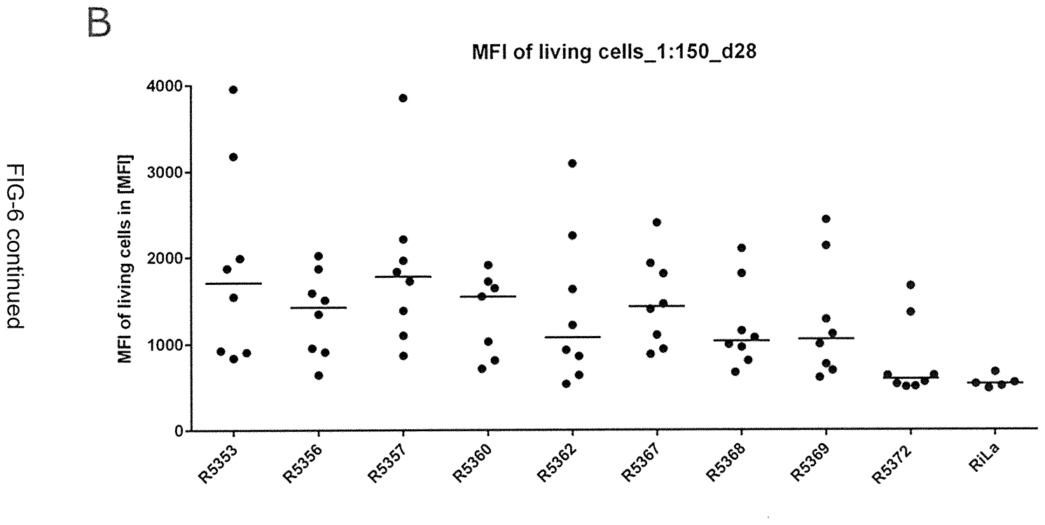

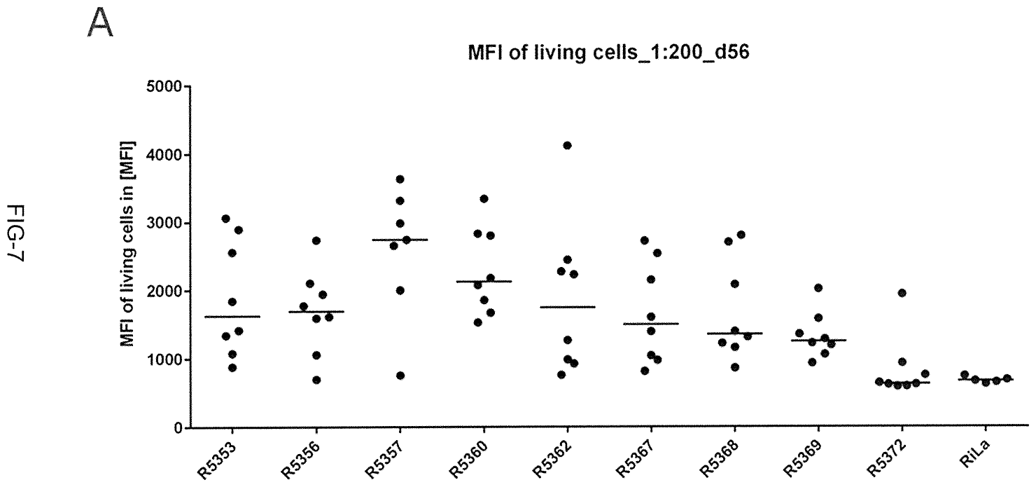

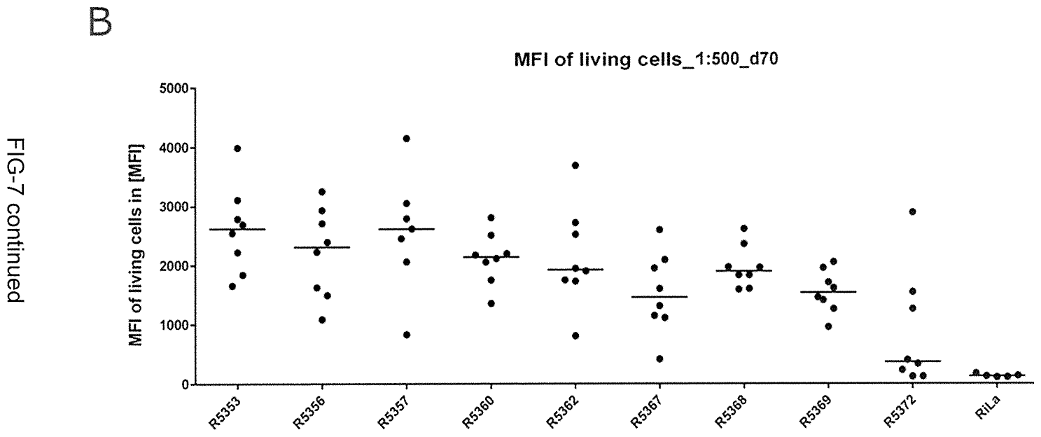

Hela cells were transfected with 2 ug YFV prME mRNA constructs (R3758) using lipofectamine. The cells were harvested 20h post transfection, and seeded at 1×10⁵ per well into a 96 well plate. The cells were incubated with corresponding sera of vaccinated mice (serum of day 14, diluted 1:100; serum of day 28, diluted 1:150; serum of day 56, diluted 1:200; serum of day 70, diluted 1:500) followed by a FITC-conjugated anti-mouse IgG antibody staining. Cells were acquired on BD FACS Canto II using DIVA software and analyzed by FlowJo. The results are shown in FIG. 6 (day 14 and day 28) and FIG. 7 (day 56 and day 70). As read out MFI of living cells (MFI=geometric mean fluorescence intensity) was used.

### 4.3. Results:

As shown in FIG. 6 and FIG. 7, the mRNA constructs encoding YFV prME harboring different UTR combinations of the invention are expressed in mice after i.m. administration. Moreover, as specific antigen IgGs were detected in sera of immunized mice, the results also show that the applied mRNA constructs are suitable to induce specific humoral immune responses. Furthermore, the results reveal that the mRNA constructs harboring different UTR combinations (HSD17B4/CASP1, Slc7a3/Ndufa1, ATP5A1/PSMB3, Nosip/CASP1, Rpl31/PSMB3, Slc7a3/CASP1, Slc7a3/COX6B1, Ndufa4/PSMB3) induce stronger immune responses compared to the reference mRNA construct (RPL32/ALB7) showing that these improved mRNA constructs may be particularly suitable for use as a vaccine, e.g. as an LNP-formulated mRNA based YFV vaccine (tested in Example 5).

### Example 5: Vaccination of Mice with LNP-Formulated mRNA Encoding YFV prME

The present example shows that LNP formulated mRNA vaccine efficiently induces binding Antibody titers in vaccinated mice at a low dose (paragraph 5.1). In addition, different mRNA constructs with optimized UTR combinations are tested as LNP-formulated vaccine (paragraph 5.4).

### 5.1. Immunization Procedure of the Dose Finding Experiment:

Female BALB/c mice (8 animals per group) were injected intramuscularly (i.m.) with LNP-formulated vaccine. As a negative control, one group of mice (5 animals) was injected with 0.9% NaCl buffer. All animals were injected on day 0 and 21. Blood samples were collected on day 21 and 35 for the determination of antibody titers. Further details are provided in Table 5 below.

TABLE 5
Vaccination regimen (Example 5.1):

| | RNA ID/SEQ | | Formu- | Dose per |
|---|---|---|---|---|
| Group | ID NO: | Construct | lation | injection |
| A | R6711/1194 | X-SS-prME-XX (opt1) | LNP | 10 ug |
| B | R6711/1194 | X-SS-prME-XX (opt1) | LNP | 5 ug |
| C | R6711/1194 | X-SS-prME-XX (opt1) | LNP | 1 ug |
| D | buffer | | | |

### 5.2 Detection of Antigen Specific Humoral Immune Responses:

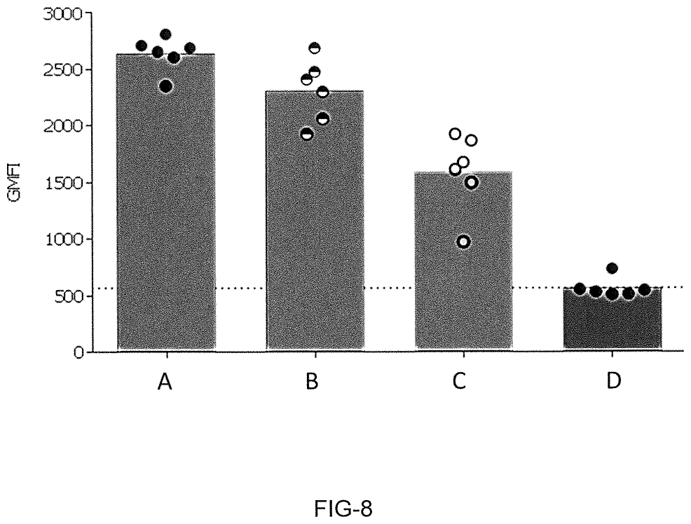

HeLa cells were transfected with 2 ug R6711 using lipofectamine. Transfected cells were harvested 20h post transfection and seeded at 1×10^{5/}well into 96-well V-bottom plate. Cells were stained with live/dead marker, fixed, permeabilized, and subsequently incubated with sera of mRNA vaccinated mice (diluted 1:50) followed by FITC-conjugated anti-mouse IgG antibody. Cells were acquired on BD FACS Canto II using DIVA software and analyzed by FlowJo. The result is shown in FIG. 8. The results show that the LNP-formulated mRNA vaccine induces binding antibody titers at a dose of 1 ug.

### 5.3. Plaque Reduction Neutralization Test (PRNT50):

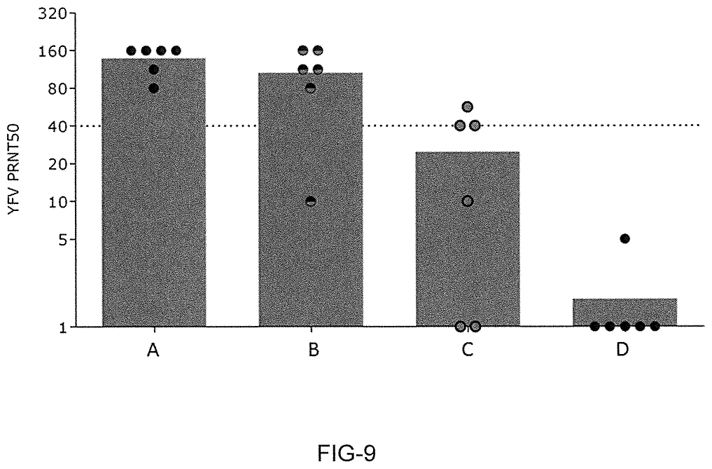

Sera are analyzed by a plaque reduction neutralization test (PRNT50), performed as commonly known in the art. Briefly, obtained serum samples of vaccinated mice were incubated with YFV. That mixture is used to infect cultured cells, and the reduction in the number of plaques was determined. The result of the PRNT assay is shown in FIG. 9. The result shows that the LNP-formulated mRNA vaccine led to the induction of YF-specific neutralizing antibodies.

5.4. Immunization Procedure of the mRNA Construct Evaluation Experiment:

Optimized YF mRNA constructs with inventive UTR combinations are used in the present experiment. Female BALB/c mice (6 animals per group) are injected intramuscularly (i.m.) with LNP-formulated mRNA with constructs as indicated in Table 6A and B. As a negative control, one group of mice is injected with buffer (ringer lactate). All animals are injected on day 0 and 21. Blood samples were collected on day 21 and 35 for the determination of antibody titers (as e.g. explained in paragraph 5.2). Splenocytes were isolated on day 35 for analysis of CD4/CD8 T cells. Further details are provided in Table 6A and B below.

TABLE 6A
Vaccination regimen (Example 5.4):

| | mRNA vaccine | | | | |
|---|---|---|---|---|---|
| Gr. Balb/C | | UTR description | RNA ID/SEQ | | Dose per |
| mice N = 8 | Construct | (5′-UTR/3′-UTR) | ID NO: | Formulation | injection |
| 1 | X-SS-prME-SS (opt1) | HSD17B4/CASP1 | R7232/654 | LNP | 5 ug |
| 2 | X-SS-prME-SS (opt1) | HSD17B4/CASP1 | R7232/654 | LNP | 1 ug |
| 3 | X-SS-prME-SS (opt1) | ATP5A1/PSMB3 | R7231/714 | LNP | 5 ug |
| 4 | X-SS-prME-SS (opt1) | ATP5A1/PSMB3 | R7231/714 | LNP | 1 ug |
| 5 | X-SS-prME-SS (opt1) | NOSIP/PSMB3 | R7230/1254 | LNP | 5 ug |
| 6 | X-SS-prME-SS (opt1) | NOSIP/PSMB3 | R7230/1254 | LNP | 1 ug |
| 7 | X-SS-prME-SS (opt1) | HSD17B4/PSMB3 | R7233/534 | LNP | 5 ug |
| 8 | X-SS-prME-SS (opt1) | HSD17B4/PSMB3 | R7233/534 | LNP | 1 ug |
| 9 | buffer | | | | |

TABLE 6B
Vaccination regimen (Example 5.4):

| | mRNA vaccine | | | | |
|---|---|---|---|---|---|
| Gr. Balb/C | | UTR description | RNA ID/SEQ | | Dose per |
| mice N = 8 | Construct | (5′-UTR/3′-UTR) | ID NO: | Formulation | injection |
| 1 | X-SS-prME-SS (opt2) | —/muag | R7250/1212 | LNP | 5 ug |
| 2 | X-SS-prME-SS (opt2) | —/muag | R7250/1212 | LNP | 1 ug |
| 3 | X-SS-prME-SS (opt4) | —/muag | R7251/1224 | LNP | 5 ug |
| 4 | X-SS-prME-SS (opt4) | —/muag | R7251/1224 | LNP | 1 ug |
| 5 | X-SS-prME-SS (opt6) | —/muag | R7252/1236 | LNP | 5 ug |
| 6 | X-SS-prME-SS (opt6) | —/muag | R7252/1236 | LNP | 1 ug |
| 7 | X-SS-prME-SS (opt11) | —/muag | R7253/1242 | LNP | 5 ug |
| 8 | X-SS-prME-SS (opt11) | —/muag | R7253/1242 | LNP | 1 ug |
| 9 | buffer | | | | |

### Example 6: In Vitro Expression Analysis of YFV mRNA Constructs Comprising Cap1 or Cap0 with Western Blot

The present Example shows that the use of Cap1 according to the invention strongly improve the expression performance of said mRNA construct compared to a reference mRNA construct comprising Cap0.

### 6.1. Western Blot Analysis

To determine in vitro protein expression performance of YFV prME mRNA constructs comprising different Cap analogues (mRNA constructs used: R7233 (cap0) and R7927 (cap1)), see Example 1, Table 3), HeLa cells were transiently transfected with said mRNA constructs and YFV prME antigen expression was analyzed in cell lysates using western blot analysis.

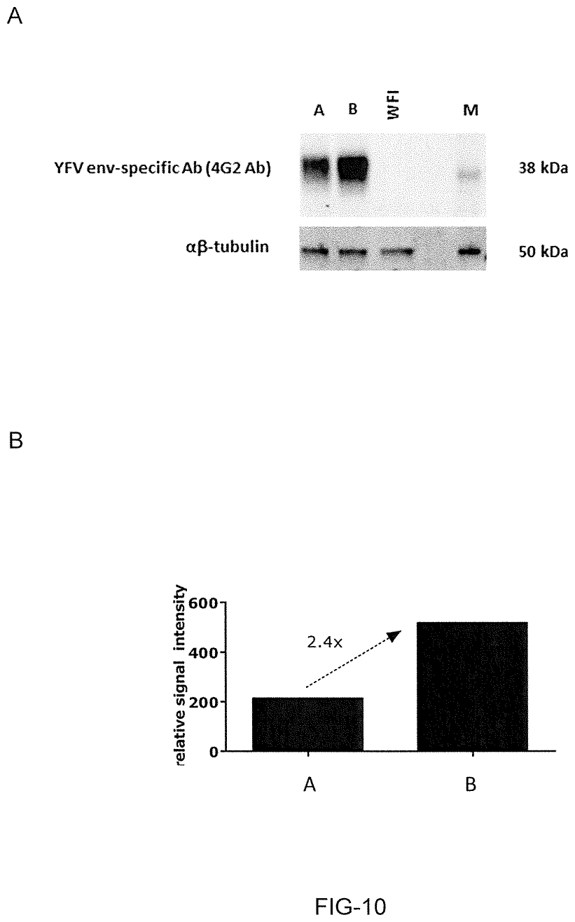

For the analysis HeLa cells were transfected with 2 μg unformulated mRNA (R7233 (cap0), R7927 (cap1) or WFI (negative control)) using 3 μl of Lipofectamine as the transfection reagent, and cell lysates were prepared 20h post transfection. Western Blot analysis was performed using anti-flavivirus group antigen (4G2; 1:2000 diluted) as primary antibody in combination with secondary anti-mouse IRDye 800CW labelled antibody. The result of the analysis is shown in FIG. 10.

### 6.2. Results:

For both of the tested mRNA constructs (R7233 (cap0) and R7927 (cap1) YFV protein was detectable. As shown in FIG. 10, the expression performances of the mRNA constructs comprising Capt according to the invention was strongly increased (2.4×) compared to the construct comprising Cap0.

### Example 7: Vaccination of Mice with LNP-Formulated mRNA Encoding YFV NS1

mRNA vaccines encoding YFV NS1 proteins (eSS-NS1, SSIgE-NS1, and eSS-NS1-Y) are prepared according to Example 1.

Female BALB/c mice or A129 mice (type-I interferon receptor deficient) (9-10 animals per group) are injected intramuscularly (i.m.) with LNP-formulated mRNA with constructs as indicated in Table 7. As a negative control, one group of mice is injected with LNP-formulated irrelevant mRNA. All animals are injected on day 0 and 21. Blood samples are collected on day 21 and 35 for the determination of antibody titers (as e.g. explained in paragraph 5.2) and NS1-specific antibodies will be detected via ELISA. Splenocytes are isolated on day 35 for analysis of CD4/CD8 T cells. A129 mice are challenged 2 weeks post last vaccination with YFV BeH 622205 strain (human case from Brazil, 2000), 10⁴ PFU via s.c. into foodpad. Upon challenge the mice are observed for 2 weeks regarding survival, body weight, morbidity index, temperature, and viremia.

TABLE 7
Vaccination regimen (Example 7):

| | mRNA vaccine | | | | |
|---|---|---|---|---|---|
| Gr. Balb/C | | UTR description | | | Dose per |
| mice N = 8 | Construct | (5′-UTR/3′-UTR) | RNA ID | Formulation | injection |
| 1 | eSS-NS1 | —/muag | R8504/R8505 | LNP | 1 μg, |
| | | | | | 2.5 μg or 5 μg |
| 2 | SSIgE-NS1 | —/muag | R8507/R8508 | LNP | 1 μg, |
| | | | | | 2.5 μg or 5 μg |
| 3 | eSS-NS1-Y | —/muag | R8501/R8502 | LNP | 1 μg, |
| | | | | | 2.5 μg or 5 μg |
| 4 | eSS-NS1 | HSD17B4/PSMB3 | R8506 | LNP | 1 μg, |
| | | | | | 2,5 μg or 5 μg |
| 5 | SSIgE-NS1 | HSD17B4/PSMB3 | R8509 | LNP | 1 μg, |
| | | | | | 2.5 μg or 5 μg |
| 6 | eSS-NS1-Y | HSD17B4/PSMB3 | R8503 | LNP | 1 μg, |
| | | | | | 2.5 μg or 5 μg |
| 7 | Irrelevant mRNA | | | LNP | 1 μg, |
| | | | | | 2.5 μg or 5 μg |

### Example 8: Vaccination of Mice with LNP-Formulated mRNA Encoding YFV prME and YFV NS1

To broaden and optimize the YFV specific immune response and to potentially reduce the pathogenicity of the YFV, mRNA vaccines encoding different YFV proteins (prME construct: SS-prME, X-SS-prME-XX, SSjev-prME, SSjev-prME-XX, SSIgE-prME, SSIgE-prME-XX, X-SS-prMEdelstem_TM-JEV, SSjev(V3)-prMEdelstem_TM-JEV, X-SS-prME and NS1 construct: eSS-NS1, SSIgE-NS1, and eSS-NS1-Y) are prepared according to Example 1.

In order to assess the effect of single or combined vaccines, these vaccines are administered i.m. with 2.5 ug mRNA for each antigen either alone or in combination as shown in Table 8.

TABLE 8
Vaccination regimen (Example 7):

| Gr. Balb/C | | UTR description | Formu- |
|---|---|---|---|
| mice N = 8 | Construct | (5′-UTR/3′-UTR) | lation |
| 1 | NS1 construct | —/muag or HSD17B4/PSMB3 | LNP |
| 2 | prME construct | —/muag or HSD17B4/PSMB3 | LNP |
| 3 | prME | —/muag or HSD17B4/PSMB3 | LNP |
| | construct + | | |
| | NS1 construct | | |
| 8 | Irrelevant | —/muag or HSD17B4/PSMB3 | LNP |
| | mRNA | | |

Female BALB/c mice or A129 mice (type-I interferon receptor deficient) (9-10 animals per group) are injected intramuscularly (i.m.) with LNP-formulated mRNA with constructs as indicated in Table 8. As a negative control, one group of mice is injected with buffer (ringer lactate). All animals are injected on day 0 and 21. Blood samples are collected on day 21 and 35 for the determination of antibody titers (as e.g. explained in paragraph 5.2) and YF-specific neutralizing antibodies are determined using the plaque reduction neutralization test (PRNT50) (as e.g. explained in paragraph 5.3) and NS1-specific antibodies are determined using ELISA, Splenocytes are isolated on day 35 for CD4/CD8 T cells analysis by ICS. A129 mice are challenged 2 weeks post last vaccination with YFV BeH 622205 strain (human case from Brazil, 2000), 10⁴ PFU via s.c. into foodpad. Upon challenge the mice are observed for 2 weeks regarding survival, body weight, morbidity index, temperature, and viremia.

### Example 9: Clinical Development of a YFV mRNA Vaccine Composition

To demonstrate safety and efficiency of the YFV mRNA vaccine composition, a clinical trial (phase I) is initiated. For clinical development, RNA is used that has been produced under GMP conditions (e.g. using a procedure as described in WO2016/180430).

In the clinical trial, a cohort of healthy human volunteers is intramuscularly injected for at least two times with respective LNP formulated vaccine compositions comprising favorable UTR combinations.

In order to assess the safety profile of the vaccine compositions according to the invention, subjects are monitored after administration (vital signals, vaccination site tolerability assessments, hematologic analysis).

The efficacy of the immunization is analyzed by determination of virus neutralizing titers (VNT) in sera from vaccinated subjects. Blood samples are collected on day 0 as baseline and after completed vaccination. Sera are analyzed for virus neutralizing antibodies.

1. An artificial RNA comprising
a) at least one heterologous 5′ untranslated region (5′-UTR) and/or at least one heterologous 3′ untranslated region (3′-UTR); and
b) at least one coding sequence operably linked to said 3′-UTR and/or 5′-UTR encoding at least one antigenic peptide or protein derived from a Yellow fever virus prME polyprotein or a fragment or variant thereof or a Yellow fewer virus NS1 protein or a fragment or variant thereof.

2. Artificial RNA according to claim 1, wherein the at least one heterologous 3′-UTR comprises a nucleic acid sequence derived from a 3′-UTR of a gene selected from an ALB7 gene, an alpha-globin gene, a PSMB3 gene, a CASP1 gene, a COX6B1 gene, a NDUFA1 gene, or from a homolog, a fragment or a variant thereof.

3. Artificial RNA according to claim 1, wherein the at least one heterologous 5′-UTR comprises a nucleic acid sequence derived from a 5′-UTR of a gene selected from a RPL32 gene, a HSD17B4 gene, a ATP5A1 gene, a NDUFA4 gene, a NOSIP gene, RPL31 gene, a SLC7A3 gene, or from a homolog, a fragment or variant of any one of these genes.

4. Artificial RNA according to any one of the preceding claims, comprising
a-1. at least one 5′-UTR derived from a 5′-UTR of a HSD17B4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
a-2. at least one 5′-UTR derived from a 5′-UTR of a NDUFA4 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
a-4. at least one 5′ UTR derived from a 5′UTR of a NOSIP gene, or from a corresponding RNA sequence, homolog, fragment or variant thereof and at least one 3′ UTR derived from a 3′UTR of a PSMB3 gene, or from a corresponding RNA sequence, homolog, fragment or variant thereof; or
b-4. at least one 5′-UTR derived from a 5′-UTR of a HSD17B4 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
c-5. at least one 5′-UTR derived from a 5′-UTR of a ATP5A1 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
d-1. at least one 5′-UTR derived from a 5′-UTR of a RPL31 gene, or from a corresponding RNA sequence, homolog, fragment or variant thereof and at least one 3′-UTR derived from a 3′-UTR of a PSMB3 gene, or from a corresponding RNA sequence, homolog, fragment or variant thereof; or
d-5. at least one 5′-UTR derived from a 5′-UTR of a SLC7A3 gene, or from a corresponding RNA sequence, homolog, fragment or variant thereof and at least one 3′-UTR derived from a 3′-UTR of a NDUFA1 gene, or from a corresponding RNA sequence, homolog, fragment or variant thereof; or
g-4. at least one 5′-UTR element derived from a 5′-UTR of a NOSIP gene, or from a corresponding RNA sequence, homolog, fragment or variant thereof and at least one 3′-UTR element derived from a 3′-UTR of a CASP1 gene, or from a corresponding RNA sequence, homolog, fragment or variant thereof; or
h-4. at least one 5′-UTR derived from a 5′-UTR of a SLC7A3 gene, or from a corresponding RNA sequence, homolog, fragment or variant thereof and at least one 3′-UTR derived from a 3′-UTR of a CASP1 gene, or from a corresponding RNA sequence, homolog, fragment or variant thereof; or
h-5. at least one 5′-UTR derived from a 5′-UTR of a SLC7A3 gene, or from a corresponding RNA sequence, homolog, fragment or variant thereof and at least one 3′-UTR derived from a 3′-UTR of a COX6B1 gene, or from a corresponding RNA sequence, homolog, fragment or variant thereof; or
i-2. at least one 5′-UTR derived from a 5′-UTR of a RPL32 gene, or from a corresponding RNA sequence, homolog, fragment or variant thereof and at least one 3′-UTR derived from a 3′-UTR of a ALB7 gene, or from a corresponding RNA sequence, homolog, fragment or variant thereof.
i-3. at least one 3′-UTR derived from a 3′-UTR of a alpha-globin gene, or from a corresponding RNA sequence, homolog, fragment or variant thereof.

5. Artificial RNA according to claim 4 comprising UTR elements according to a-1 (HSD17B4/PSMB3), a-4 (NDUFA4/PSMB3), b-4 (HSD17B4/CASP1), c-5 (ATP5A1/PSMB3), or g-4 (NOSIP/CASP1).

6. Artificial RNA according to any one of the preceding claims, wherein
said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1, 2 or a fragment or a variant thereof;
said 5′-UTR derived from a ATP5A1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 5, 6 or a fragment or a variant thereof;
said 5′-UTR derived from a NDUFA4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 3, 4 or a fragment or a variant thereof;
said 5′-UTR derived from a NOSIP gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 7, 8 or a fragment or a variant thereof;
said 5′-UTR derived from a RPL31 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 9, 10 or a fragment or a variant thereof;
said 5′-UTR derived from a RPL32 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 13, 14 or a fragment or a variant thereof;
said 5′-UTR derived from a SLC7A3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 11, 12 or a fragment or a variant thereof;
said 3′-UTR derived from a PSMB3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 15, 16 or a fragment or a variant thereof;
said 3′-UTR derived from a CASP1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 17, 18 or a fragment or a variant thereof;
said 3′-UTR derived from a COX6B1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 21, 22 or a fragment or a variant thereof;
said 3′-UTR derived from a NDUFA1 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 19, 20 or a fragment or a variant thereof;
said 3′-UTR derived from a ALB7 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 23, 24 or a fragment or a variant thereof;
said 3′-UTR derived from a alpha-globin gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 25, 26 or a fragment or a variant thereof.

7. Artificial RNA according to any one of the preceding claims, wherein the at least one antigenic peptide or protein derived from a Yellow fever virus prME polyprotein is pr, M, E, ME, prM or prME, or a fragment or variant of any of these.

8. Artificial RNA according to any one of the preceding claims, wherein the at least one antigenic peptide or protein is prME or prME additionally comprising a C-terminal overhang comprising a fragment of YFV non-structural protein NS1 and/or an N-terminal overhang comprising a fragment of YFV capsid protein C.

9. Artificial RNA according to any one of the preceding claims, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 120-126 or a fragment or variant of any of these sequences.

10. Artificial RNA according to any one of the preceding claims, wherein the at least one coding sequence additionally encodes at least one heterologous signal sequence, preferably signal sequence derived from IgE or Japanese encephalitis virus (JEV), or preferably selected from SEQ ID NOs: 56-61, 1330-1357 or a fragment or variant of any of these sequences.

11. Artificial RNA according to any one of the preceding claims, wherein the at least one coding sequence additionally encodes at least one heterologous further virus element, preferably a JEV stem sequence, preferably selected from SEQ ID NO: 110 or a fragment or variant of any of these sequences.

12. Artificial RNA according to any one of the preceding claims, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 127-157, 1587, 1588 or a fragment or variant of any of these sequences.

13. Artificial RNA according to claims 1-6, wherein the at least one antigenic peptide or protein is NS1 or NS1 additionally comprising a C-terminal overhang comprising a fragment of YFV non-structural protein NS2A and/or an N-terminal overhang comprising a fragment of YFV envelope protein E.

14. Artificial RNA according to claims 1-6 and 13, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 2201-2204 or a fragment or variant of any of these sequences.

15. Artificial RNA according to claims 1-6 and 13-14, wherein the at least one coding sequence additionally encodes at least one heterologous signal sequence, preferably signal sequence derived from IgE or Japanese encephalitis virus (JEV), preferably selected from SEQ ID NOs: 56-61, 1330-1356 or a fragment or variant of any of these sequences.

16. Artificial RNA according to claim 15, wherein the at least one coding sequence encodes at least one of the amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 2005 or a fragment or variant of thereof.

17. Artificial RNA according to any one of the preceding claims, wherein the at least one coding sequence is located between said 5′-UTR and said 3′-UTR, preferably downstream of said 5′-UTR and upstream of said 3′-UTR.

18. Artificial RNA according to any one of the preceding claims, wherein the at least one coding sequence comprises at least one of the nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 158-527, 1589-1608, 2206-2245 or a fragment or a fragment or variant of any of these sequences.

19. Artificial RNA according to any one of the preceding claims, wherein the artificial RNA is a modified and/or stabilized artificial RNA.

20. Artificial RNA according to any one of the preceding claims, wherein the at least one coding sequence is a codon modified coding sequence, wherein the amino acid sequence encoded by the at least one codon modified coding sequence is preferably not being modified compared to the amino acid sequence encoded by the corresponding wild type coding sequence.

21. Artificial RNA according to claim 20, wherein the at least one codon modified coding sequence is selected from C maximized coding sequence, CAI maximized coding sequence, human codon usage adapted coding sequence, G/C content modified coding sequence, and G/C optimized coding sequence, or any combination thereof.

22. Artificial RNA according to claim 20 or 21, wherein the at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 195-527, 1591-1608, 2211-2245 or a fragment or variant of any of these sequences.

23. Artificial RNA according to claim 22, wherein the at least one coding sequence comprises a codon modified coding sequence comprising a nucleic acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 195-305, 1591-1596, 2211-2215 or a fragment or variant of any of these sequences.

24. Artificial RNA according to any one of the preceding claims, wherein the RNA is an mRNA, a self-replicating RNA, a circular RNA, or a replicon RNA.

25. Artificial RNA according to claim 24, wherein the RNA is an mRNA.

26. Artificial RNA according to any one of the preceding claims, which comprises a 5′-cap structure, preferably m7G, cap0, cap1 or cap2.

27. Artificial RNA according to any one of the preceding claims, which comprises at least one histone stem-loop, wherein the histone stem-loop preferably comprises a nucleic acid sequence according to SEQ ID NOs: 27, 28 or a fragment or variant thereof.

28. Artificial RNA according to any one of the preceding claims which comprises a 3′-terminal sequence element according to SEQ ID NOs: 32-51, 1323-1329 or a fragment or variant thereof.

29. Artificial RNA according to any one of the preceding claims comprising, preferably in 5′- to 3′-direction, the following elements a) to h):
a) 5′-cap structure, preferably as defined in claim 26;
b) optionally, 5′-UTR, preferably as defined by any one of claim 3 or 6;
c) at least one coding sequence, preferably as defined by any one of claims 9 to 23;
d) 3′-UTR, preferably as defined by any one of claim 2 or 6;
e) optionally, a poly(A) sequence;
f) optionally, a poly(C) sequence,
g) optionally, a histone stem-loop, preferably as defined by any one of claim 27;
h) optionally, a 3′-terminal sequence element as defined by claim 28.

30. Artificial RNA according claims 1 to 29 comprising the following elements:
a) 5′-cap structure, preferably as defined in claim 26;
b) a 5′-UTR and a 3′-UTR according to a-1, a-2, a-4, b-4, c-5, d-1, d-5, g-4, h-4, h-5, i-2, or i-3;
c) at least one coding sequence as defined in claims 9 to 23, wherein said coding sequence is located between said 5′-UTR and said 3′-UTR, preferably downstream of said 5′-UTR and upstream of said 3′-UTR.
e) optionally, a poly(A) sequence
f) optionally, poly(C) sequence
g) optionally, histone stem-loop, preferably as defined by any one of claim 27;
h) optionally, a 3′-terminal sequence element as defined by claim 28.

31. Artificial RNA according claims 1 to 30 comprising the following elements:
a) 5′-cap structure, preferably as defined in claim 26;
b) a 5′-UTR and a 3′-UTR according to a-1, a-4, b-4, c-5, or g-4;
c) at least one coding sequence as defined in claims 9 to 23, wherein said coding sequence is located between said 5′-UTR and said 3′-UTR, preferably downstream of said 5′-UTR and upstream of said 3′-UTR.
d) optionally, a poly(A) sequence,
e) optionally, poly(C) sequence,
f) optionally, histone stem-loop, preferably as defined by any one of claim 27;
g) optionally, a 3′-terminal sequence element as defined by claim 28.

32. Artificial RNA according to any one of claims 25 to 31, wherein the artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 528-1320, 1609-2200, 2246-2593 or a fragment or variant of any of these sequences.

33. Artificial RNA according to claim 32, wherein
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 528-587, 1308, 1609-1648, 2129-2134, 2141-2146, 2153-2158, 2165-2170, 2177-2182, 2189-2194, 2246-2269, 2558-2560, 2576-2578, 2564-2566, 2582-2584, 2570-2572, 2588-2590 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 1248-1307, 1320, 2089-2128, 2534-2557 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 648-707, 1310, 1689-1728, 2294-2317 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 708-767, 1311, 1729-1768, 2318-2341 or a fragment or variant of any of these sequences;
said artificial RNA comprises or consists of an RNA sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 888-947, 1314, 1849-1888, 2390-2413 or a fragment or variant of any of these sequences; or
said artificial RNA comprises or consists of an RNA sequence which is identical or at least 70%, 80%, 85%, 86%, 87%, 88%, 89%, 90%, 91%, 92%, 93%, 94%, 95, 96%, 97%, 98%, or 99% identical to a nucleic acid sequence selected from the group consisting of SEQ ID NOs: 1188-1247, 1319, 2049-2088, 2135-2140, 2147-2152, 2159-2164, 2171-2176, 2183-2188, 2195-2200, 2510-2533, 2561-2563, 2579-2581, 2567-2569, 2585-2587, 2573-2575, 2591-2593 or a fragment or variant of any of these sequences.

34. A composition comprising at least one artificial RNA as defined in any one of claims 1 to 33, wherein the composition optionally comprises at least one pharmaceutically acceptable carrier.

35. Composition according to claim 34, wherein the composition comprises at least one YFV prME RNA construct, preferably selected from X-SS-prME-XX, X-SS-prME, SS-prME, SSjev(V3)-prME-XX, X-SS-prMEdelstem_TM-JEV or SSjev(V3)-prMEdelstem_TM-JEV and, in addition, at least one YFV NS1 RNA construct, preferably selected from eSS-NS1-Y, eSS-NSE and SSIgE-NS1.

36. Composition according to claim 35 or 34, wherein the at least one artificial RNA is complexed or associated with or at least partially complexed or partially associated with one or more cationic or polycationic compound, preferably cationic or polycationic polymer, cationic or polycationic polysaccharide, cationic or polycationic lipid, cationic or polycationic protein, cationic or polycationic peptide, or any combinations thereof.

37. Composition according to claim 36, wherein the cationic or polycationic peptide is protamine.

38. Composition according to any one of claim 36 or 37 comprising the at least one artificial RNA, which is complexed with one or more cationic or polycationic compounds, preferably protamine, and at least one free artificial RNA.

39. Composition according to claim 36, wherein the cationic or polycationic peptide is selected from CHHHHHHRRRRHHHHHHC (SEQ ID NO: 55), CRRRRRRRRRRRRC (SEQ ID NO: 52), CRRRRRRRRRRRR (SEQ ID NO: 53), or WRRRRRRRRRRRRC (SEQ ID NO: 54), or a fragment or variant of any of these sequences.

40. Composition according to claim 36, wherein the cationic or polycationic polymer is a polyethylene glycol/peptide polymer selected from HO-PEG5000-S-(S-CHHHHHHRRRRHHHHHHC-S-)7-S-PEG5000-OH (SEQ ID NO: 55 of the peptide monomer), HO-PEG5000-S-(S-CHHHHHHRRRRHHHHHHC-S-)4-S-PEG5000-0H (SEQ ID NO: 55 as peptide monomer), HO-PEG5000-S-(S-CGHHHHHRRRRHHHHHGC-S-)7-S-PEG5000-0H (SEQ ID NO: 1321 as peptide monomer), or HO-PEG5000-S-(S-CGHHHHHRRRRHHHHHGC-S-)4-S-PEG5000-0H (SEQ ID NO: 1321 as peptide monomer).

41. Composition according to claim 40, wherein the composition comprises a lipid component, preferably a lipidoid component, wherein the lipidoid component is a compound according to formula A

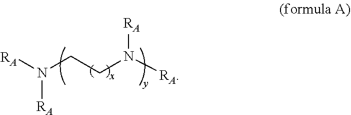

42. Composition according to claim 41, wherein the lipidoid component is 3-C12-OH according to formula B.

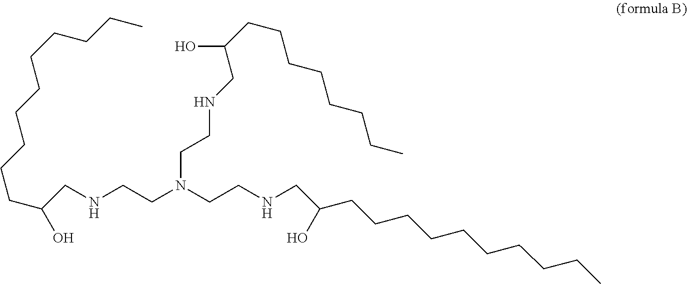 [SMILES: CCCCCCCCCCC(O)CNCCN(CCNCC(O)CCCCCCCC)CCNCC(O)CCCCCCCCCC]

43. Composition according to claim 36, wherein the artificial RNA is complexed or associated with one or more lipids, thereby forming liposomes, lipid nanoparticles, lipoplexes, and/or nanoliposomes.

44. Composition according to claim 43, wherein the artificial RNA is complexed with one or more lipids thereby forming lipid nanoparticles (LNP).

45. Composition according to claim 44, wherein the LNP comprises a cationic lipid with the formula 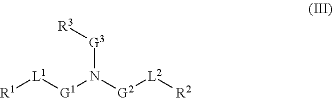
or a pharmaceutically acceptable salt, tautomer, prodrug or stereoisomer thereof, wherein:
L¹ or L² is each independently —O(C═O)—, —(C═O)O—, —C(═O)—, —O—, —S(O)X—, —S—S—, —C(═O)S—, —SC(═O)—, —NR^{a}C(═O)—, —C(═O)NR^{a}—, —NR^{a}C(═O)NR^{a}—, —OC(═O)NR^{a}— or —NR^{a}C(═O)O—, preferably L¹ or L² is —O(C═O)— or —(C═O)O—;
G¹ and G² are each independently unsubstituted C₁-C₁₂ alkylene or C₁-C₁₂ alkenylene;
G³ is C₁-C₂₄ alkylene, alkenylene, C₃-C₈ cycloalkylene, or C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2;

46. Composition according to claim 45, wherein the cationic lipid is a compound of formula III, and wherein:
L¹ and L² are each independently —O(C═O)— or (C═O)—O—;
G³ is C₁-C₂₄ alkylene or C₁-C₂₄ alkenylene; and
R³ is H or OR⁵.

47. Composition according to any one of claims 45 to 46, wherein the cationic lipid is a compound of formula III, and wherein:
L¹ and L² are each independently —O(C═O)— or (C═O)—O—; and
R¹ and R² each independently have one of the following structures:

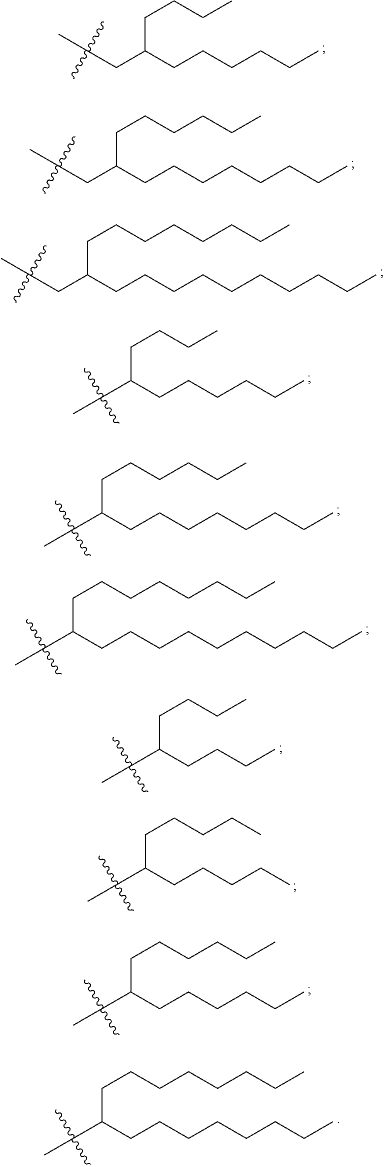

48. Composition according to any one of claims 45 to 47, wherein the cationic lipid is a compound of formula III, and wherein R³ is OH.

49. Composition according to any one of claims 45 to 48, wherein the cationic lipid is selected from structures III-1 to III-36:

| No. | Structure |
|---|---|
| III-1 | 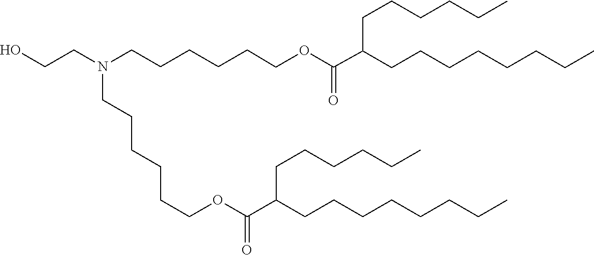 [SMILES: CCCCCCCCC(CCCCCC)C(=O)OCCCCCCN(CCO)CCCCCCOC(=O)C(CCCCCC)CCCCCCCC] |
| III-2 | 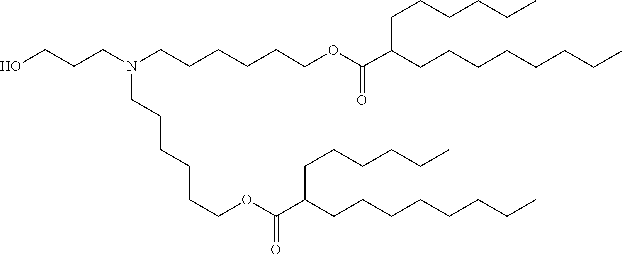 [SMILES: CCCCCCCCC(CCCCCC)C(=O)OCCCCCCN(CCCO)CCCCCCOC(=O)C(CCCCCC)CCCCCCCC] |
| III-3 | 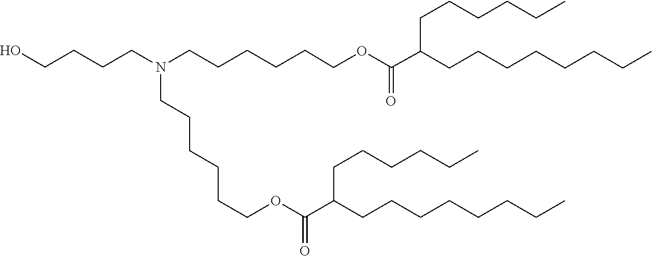 [SMILES: CCCCCCCCC(CCCCCC)C(=O)OCCCCCCN(CCCCO)CCCCCCOC(=O)C(CCCCCC)CCCCCCCC] |
| III-4 | 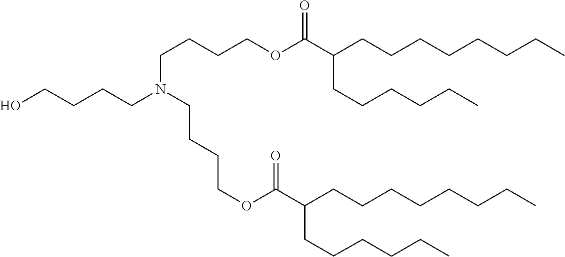 [SMILES: CCCCCCCCC(CCCCCC)C(=O)OCCCCN(CCCCO)CCCCOC(=O)C(CCCCCC)CCCCCCCC] |
| III-5 | 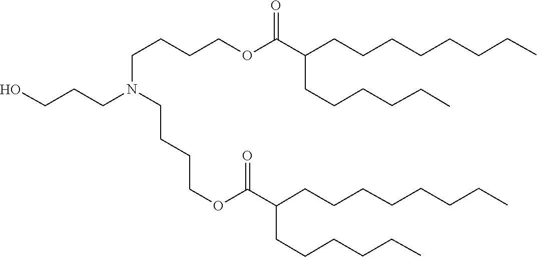 [SMILES: CCCCCCCCC(CCCCCC)C(=O)OCCCCN(CCCO)CCCCOC(=O)C(CCCCCC)CCCCCCCC] |
| III-6 | 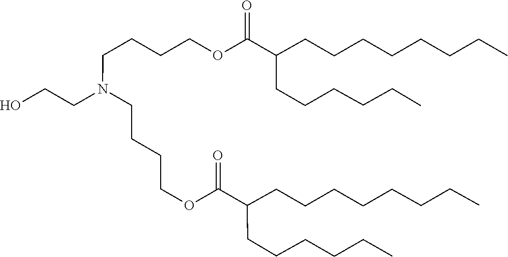 [SMILES: CCCCCCCCC(CCCCCC)C(=O)OCCCCN(CCO)CCCCOC(=O)C(CCCCCC)CCCCCCCC] |
| III-7 | 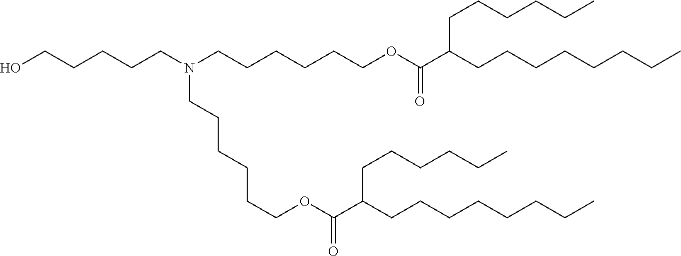 [SMILES: CCCCCCCCC(CCCCCC)C(=O)OCCCCCCN(CCCCCO)CCCCCCOC(=O)C(CCCCCC)CCCCCCCC] |
| III-8 | 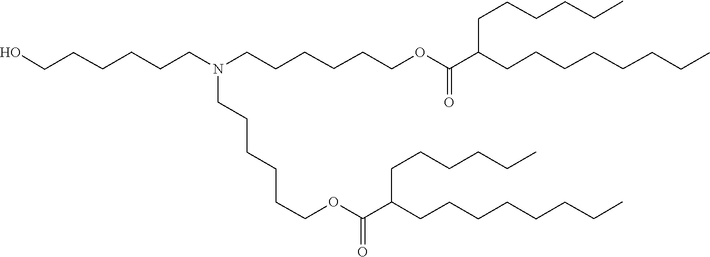 [SMILES: CCCCCCCCC(CCCCCC)C(=O)OCCCCCCN(CCCCCCO)CCCCCCOC(=O)C(CCCCCC)CCCCCCCC] |
| III-9 | 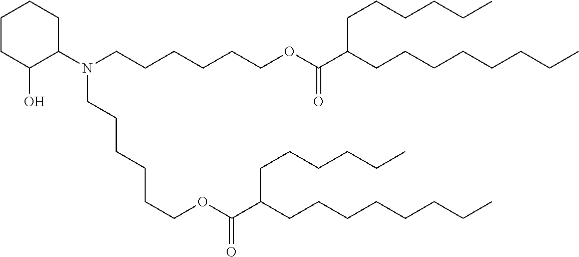 [SMILES: CCCCCCCCC(CCCCCC)C(=O)OCCCCCCN(CCCCCCOC(=O)C(CCCCCC)CCCCCCCC)C1CCCCC1O] |
| III-10 | 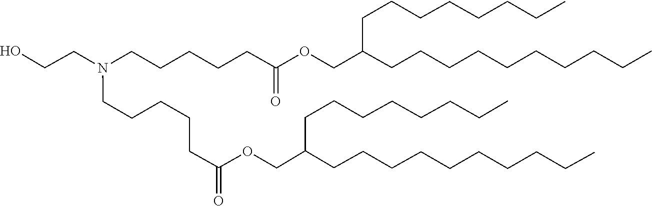 [SMILES: CCCCCCCCCCC(CCCCCCCC)COC(=O)CCCCCN(CCO)CCCCCC(=O)OCC(CCCCCCCC)CCCCCCCCCC] |
| III-11 | 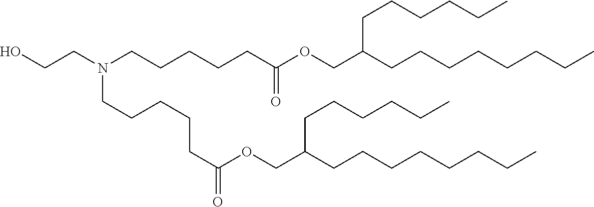 [SMILES: CCCCCCCCC(CCCCCC)COC(=O)CCCCCN(CCO)CCCCCC(=O)OCC(CCCCCC)CCCCCCCC] |
| III-12 | 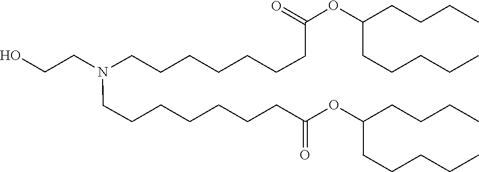 [SMILES: CCCCCC(CCCC)OC(=O)CCCCCCCN(CCO)CCCCCCCC(=O)OC(CCCC)CCCCC] |
| III-13 | 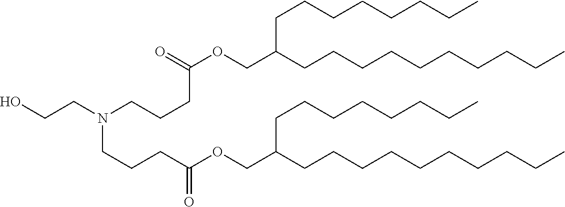 [SMILES: CCCCCCCCCCC(CCCCCCCC)COC(=O)CCCN(CCO)CCCC(=O)OCC(CCCCCCCC)CCCCCCCCCC] |
| III-14 | 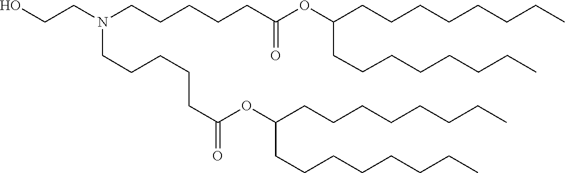 [SMILES: CCCCCCCCC(CCCCCCCC)OC(=O)CCCCCN(CCO)CCCCCC(=O)OC(CCCCCCCC)CCCCCCCC] |
| III-15 | 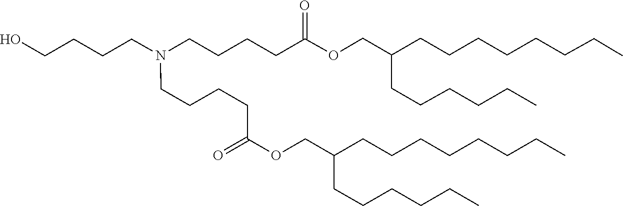 [SMILES: CCCCCCCCC(CCCCCC)COC(=O)CCCCN(CCCCO)CCCCC(=O)OCC(CCCCCC)CCCCCCCC] |
| III-16 | 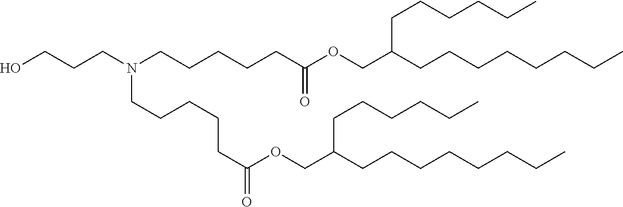 [SMILES: CCCCCCCCC(CCCCCC)COC(=O)CCCCCN(CCCO)CCCCCC(=O)OCC(CCCCCC)CCCCCCCC] |
| III-17 | 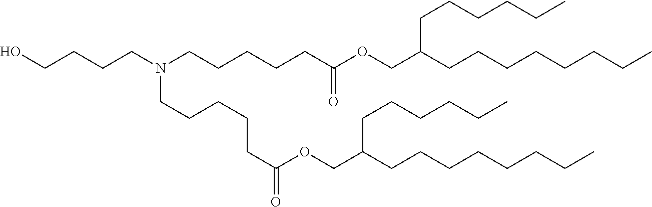 [SMILES: CCCCCCCCC(CCCCCC)COC(=O)CCCCCN(CCCCO)CCCCCC(=O)OCC(CCCCCC)CCCCCCCC] |
| III-18 | 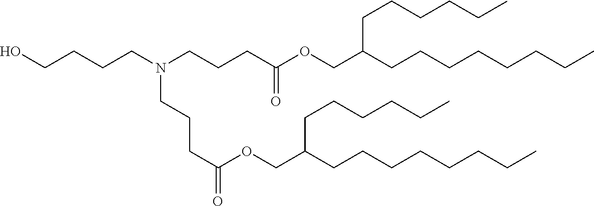 [SMILES: CCCCCCCCC(CCCCCC)COC(=O)CCCN(CCCCO)CCCC(=O)OCC(CCCCCC)CCCCCCCC] |
| III-19 | 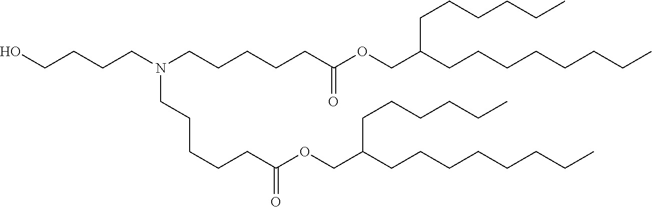 [SMILES: CCCCCCCCC(CCCCCC)COC(=O)CCCCCN(CCCCO)CCCCCC(=O)OCC(CCCCCC)CCCCCCCC] |
| III-20 | 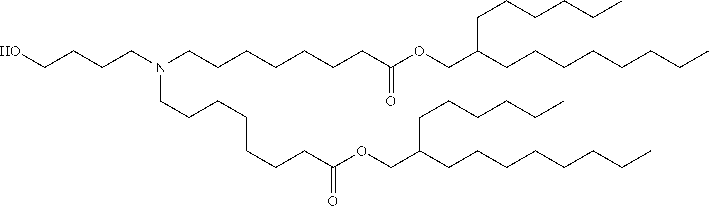 [SMILES: CCCCCCCCC(CCCCCC)COC(=O)CCCCCCCN(CCCCO)CCCCCCCC(=O)OCC(CCCCCC)CCCCCCCC] |
| III-21 | 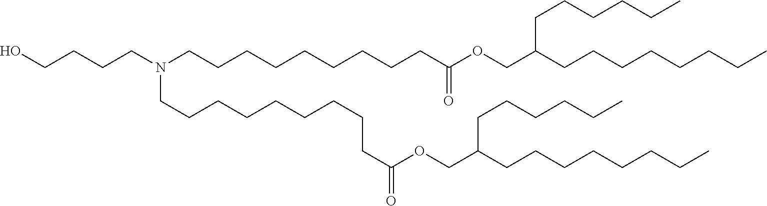 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 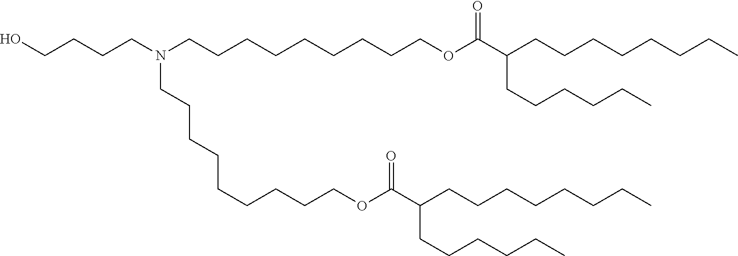 [SMILES: CCCCCCCCC(CCCCCC)C(=O)OCCCCCCCCCN(CCCCO)CCCCCCCCCOC(=O)C(CCCCCC)CCCCCCCC] |
| III-23 | 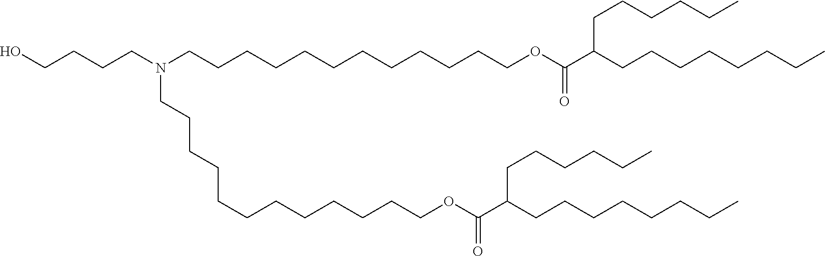 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 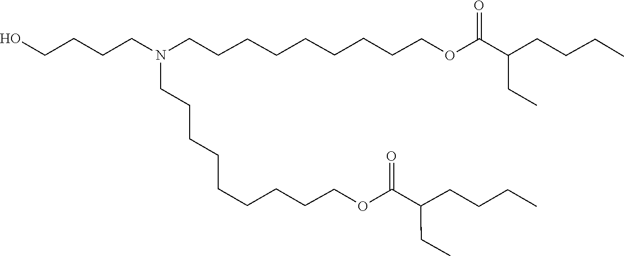 [SMILES: CCCCC(CC)C(=O)OCCCCCCCCCN(CCCCO)CCCCCCCCCOC(=O)C(CC)CCCC] |
| III-25 | 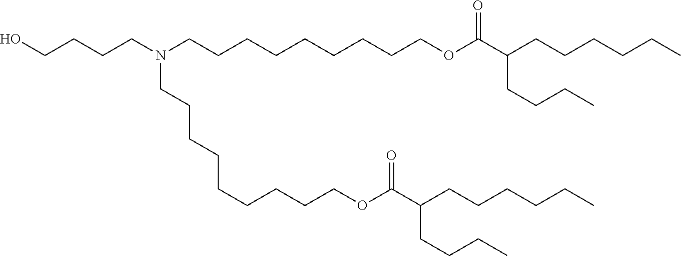 [SMILES: CCCCCCC(CCCC)C(=O)OCCCCCCCCCN(CCCCO)CCCCCCCCCOC(=O)C(CCCC)CCCCCC] |
| III-26 | 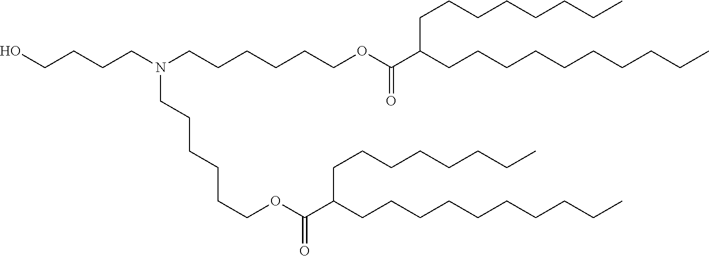 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 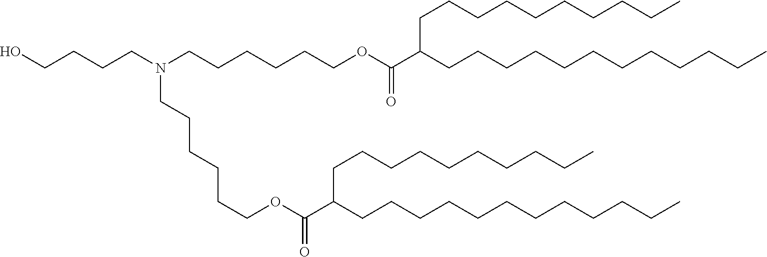 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 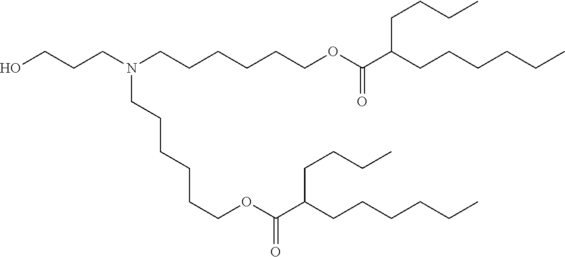 [SMILES: CCCCCCC(CCCC)C(=O)OCCCCCCN(CCCO)CCCCCCOC(=O)C(CCCC)CCCCCC] |
| III-29 | 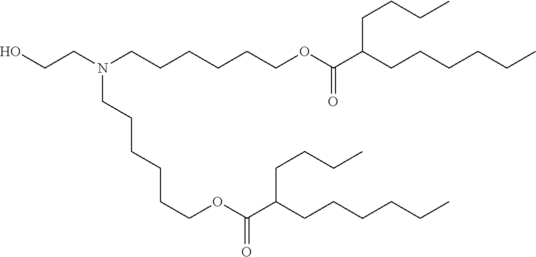 [SMILES: CCCCCCC(CCCC)C(=O)OCCCCCCN(CCO)CCCCCCOC(=O)C(CCCC)CCCCCC] |
| III-30 | 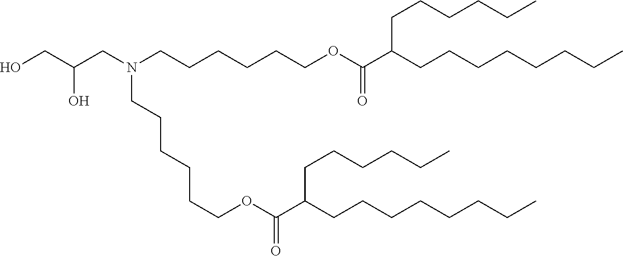 [SMILES: CCCCCCCCC(CCCCCC)C(=O)OCCCCCCN(CCCCCCOC(=O)C(CCCCCC)CCCCCCCC)CC(O)CO] |
| III-31 | 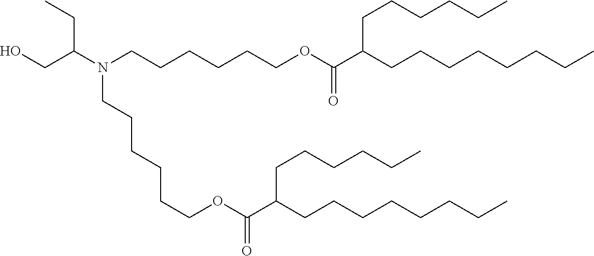 [SMILES: CCCCCCCCC(CCCCCC)C(=O)OCCCCCCN(CCCCCCOC(=O)C(CCCCCC)CCCCCCCC)C(CC)CO] |
| III-32 | 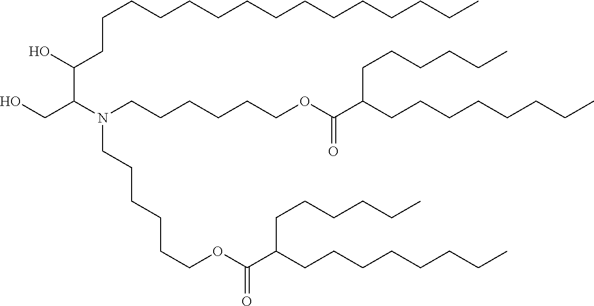 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 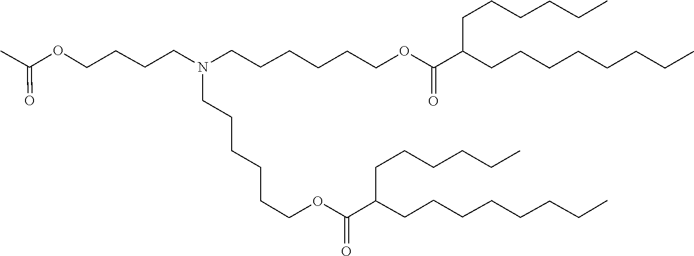 [SMILES: CCCCCCCCC(CCCCCC)C(=O)OCCCCCCN(CCCCCCOC(=O)C(CCCCCC)CCCCCCCC)CCCCOC(C)=O] |
| III-34 | 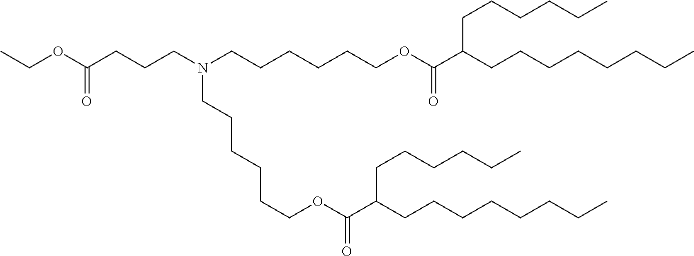 [SMILES: CCCCCCCCC(CCCCCC)C(=O)OCCCCCCN(CCCCCCOC(=O)C(CCCCCC)CCCCCCCC)CCCC(=O)OCC] |
| III-35 | 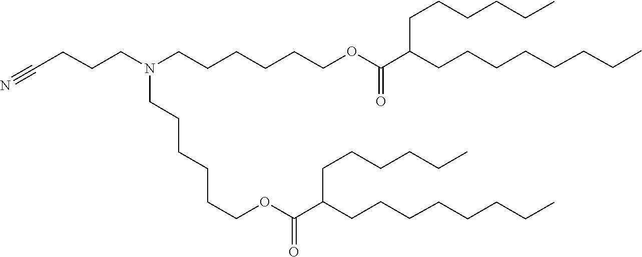 [SMILES: CCCCCCCCC(CCCCCC)C(=O)OCCCCCCN(CCCC#N)CCCCCCOC(=O)C(CCCCCC)CCCCCCCC] |
| III-36 | 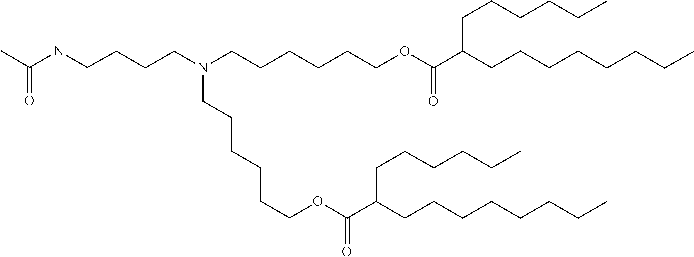 |

50. Composition according to any one of claims 45 to 49, wherein the cationic lipid is

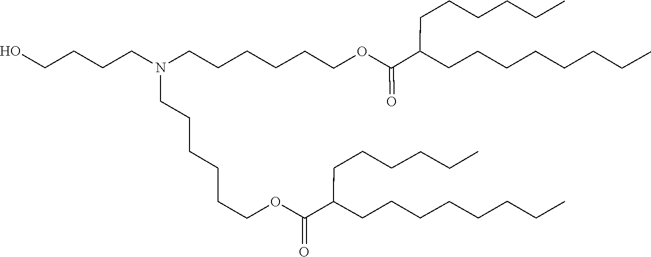 [SMILES: CCCCCCCCC(CCCCCC)C(=O)OCCCCCCN(CCCCO)CCCCCCOC(=O)C(CCCCCC)CCCCCCCC]

51. Composition according to any one of claims 44 to 50, wherein the LNP additionally comprises a PEG lipid with the formula (IV): 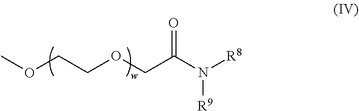
wherein R⁸ and R⁹ are each independently a straight or branched, saturated or unsaturated alkyl chain containing from 10 to 30 carbon atoms, wherein the alkyl chain is optionally interrupted by one or more ester bonds;
and w has a mean value ranging from 30 to 60.

52. Composition according to claim 51, wherein in the PEG lipid R⁸ and R⁹ are saturated alkyl chains.

53. Composition according to claim 51 or 52, wherein the PEG lipid is 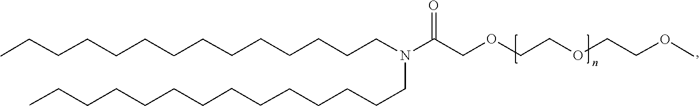
wherein n has a mean value ranging from 30 to 60, preferably wherein n has a mean value of about 45, 46, 47, 48, 49, 50, 51, 52, 53, 54, most preferably wherein n has a mean value of 49.

54. Composition according to any one of claims 44 to 53, wherein the LNP additionally comprises one or more neutral lipids and/or a steroid or steroid analogues.

55. Composition according to claim 54, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE).

56. Composition according to claim 54 or 55 wherein the neutral lipid is 1,2-distearoyl-sn-glycero-3-phosphocholine (DSPC), and wherein the molar ratio of the cationic lipid to DSPC is optionally in the range from about 2:1 to 8:1.

57. Composition according to claim 54, wherein the steroid is cholesterol, and wherein the molar ratio of the cationic lipid to cholesterol is optionally in the range from about 2:1 to 1:1

58. Composition according to any one of claims 44 to 57, wherein the LNP essentially consists of
(i) at least one cationic lipid, preferably as defined in any one of claims 47 to 50;
(ii) a neutral lipid, preferably as defined in any one of claims 54 to 56;
(iii) a steroid or steroid analogue, preferably as in claim 57; and
(iv) a PEG-lipid, e.g. PEG-DMG or PEG-cDMA, preferably as defined in any one of claims 51 to 53,
wherein (i) to (iv) are in a molar ratio of about 20-60% cationic lipid, 5-25% neutral lipid, 25-55% sterol, and 0.5-15% PEG-lipid.

59. A vaccine comprising the artificial RNA as defined in any one of claims 1 to 33, or the composition as defined in any one of claims 34 to 58.

60. Vaccine according to claim 56, wherein the artificial RNA as defined in any one of claims 1 to 33 or the composition as defined in any one of claims 34 to 58 elicits an adaptive immune response.

61. Vaccine according to claim 59 or 60, wherein the vaccine further comprises a pharmaceutically acceptable carrier and optionally at least one adjuvant.

62. A Kit or kit of parts comprising the artificial RNA as defined in any one of claims 1 to 33, the composition as defined in any one of claims 34 to 58, or the vaccine as defined in any one of claims 59 to 61, optionally comprising a liquid vehicle for solubilising, and optionally technical instructions providing information on administration and dosage of the components.

63. Kit or kit of parts according to claim 62 further comprising Ringer lactate solution.

64. Artificial RNA as defined in any one of claims 1 to 33, the composition as defined in any one of claims 34 to 58, the vaccine as defined in any one of claims 59 to 61, or the kit or kit of parts as defined in claims 62 to 63 for use as a medicament.

65. Artificial RNA as defined in any one of claims 1 to 33, the composition as defined in any one of claims 34 to 58, the vaccine as defined in any one of claims 59 to 61, or the kit or kit of parts as defined in claims 62 to 63 for use in the treatment or prophylaxis of an infection with Yellow fever virus, or a disorder related to such an infection.

66. A method of treating or preventing a disorder, wherein the method comprises applying or administering to a subject in need thereof the artificial RNA as defined in any one of claims 1 to 33, the composition as defined in any one of claims 34 to 58, the vaccine as defined in any one of claims 59 to 61, or the kit or kit of parts as defined in claims 62 to 63.

67. Method according to claim 66, wherein the disorder is an infection with a Yellow fever virus, or a disorder related to such an infection.

68. Method according to claim 66 or 67, wherein the subject in need is a mammalian subject or an avian subject.

69. Method according to claim 68, wherein the mammalian subject is a human subject.
